(19) United States
(12) Patent Application Publication
(10) Pub. No.: US 2024/0279335 A1
(43) Pub. Date: Aug. 22, 2024

(54) CD28 SHEDDING BLOCKING AGENTS

(71) Applicant: BIOND BIOLOGICS LTD., Misgav Industrial Park (IL)

(72) Inventors: Anna FRIDMAN-DROR, Kibbutz Dalia (IL); Tal GABAY, Atlit (IL); Motti HAKIM, Emek Yizrael (IL); Lilach CHEN ZELTSBURG, Nahariya (IL); Ayala LEWKOWICZ, Kibbutz Kfar Szold (IL); Ilana MANDEL, Manof (IL); Tehila BEN-MOSHE, Tel Aviv (IL); Yair SAPIR, Manof (IL); Avidor SHULMAN, Rakefet (IL)

(21) Appl. No.: 18/689,483

(22) PCT Filed: Sep. 6, 2022
(86) PCT No.: PCT/IL2022/050972

§ 371 Date: Mar. 6, 2024

Related U.S. Application Data
(60) Provisional application No. 63/241,010, filed on Sep. 6, 2021.
(60) Provisional application No. 63/241,011, filed on Sep. 6, 2021.
(60) Provisional application No. 63/347,756, filed on Jun. 1, 2022.

(51) Int. Cl. C07K 16/28; A61K 39/00; A61P 35/00

(52) U.S. Cl. CPC C07K 16/2818; A61P 35/00; A61K 2039/505; C07K 2317/22; C07K 2317/24; C07K 2317/34; C07K 2317/569; C07K 2317/76; C07K 2317/92

(57) ABSTRACT

Single domain antibodies (sdAbs) that block CD28 cleavage are provided. Dimeric agents at least two membranal CD28 binding agents are also provided, as are methods of using the sdAbs and/or dimeric agents and pharmaceutical compositions and kits comprising the sdAbs and/or dimeric agents.

### CROSS-REFERENCE TO RELATED APPLICATIONS

This application is a National Phase of PCT Patent Application No. PCT/IL2022/050972 having International filing date of Sep. 6, 2022, which claims the benefit of priority of U.S. Provisional Patent Application No. 63/241,010 filed Sep. 6, 2021, U.S. Provisional Patent Application No. 63/241,011 filed Sep. 6, 2021 and U.S. Provisional Patent Application No. 63/347,756 filed Jun. 1, 2022, the contents of the above applications are all incorporated by reference in their entirety.

### REFERENCE TO AN ELECTRONIC SEQUENCE LISTING

The contents of the electronic sequence listing (BDB-P-015-US.xml; Size: 153,006 bytes; and Date of Creation: Mar. 14, 2024) is herein incorporated by reference in its entirety.

### FIELD OF INVENTION

The present invention is in the field of immune regulation and immunotherapy.

### BACKGROUND OF THE INVENTION

The adaptive immune system plays a critical role in the regulation and protection against pathogens and cancer cells, mainly by orchestrating the stimulation of antigen specific helper CD4+ and cytotoxic CD8+ T cells. Durable and persistent activation of T cells by antigen presenting cells (APC), involves i) engagement of the T cell receptor (TCR) with peptides presented by major histocompatibility complexes (MHCs) on APC; and ii) co-stimulatory CD28 receptors on T cells binding B7-1 (CD80) and B7-2 (CD86) ligands expressed also by the APC. The biological consequences of CD28 co-stimulation are numerous and include control of the T cell cycle, expansion, differentiation, as well as amplification of TCR stimulation by lowering the threshold needed for achieving immune effector function.

International Patent Application WO2019175885 discloses that soluble CD28 (sCD28) is produced by proteolytic cleavage of the stalk domain of CD28 and active shedding of the extracellular domain of CD28 from the plasma membrane. The use of antibodies against CD28 which inhibit this shedding and thus enhance the immune response are also disclosed. The use of these antibodies in enhancing PD-1/PD-L1 based immunotherapy is also disclosed. Importantly, these antibodies also do not have a CD28 agonist or antagonist effect and thus do not indiscriminately enhance immune activation, nor inhibit activation by blocking binding of CD86 to its ligand CD28. There is a need for superior molecules that inhibit the proteolytic shedding of CD28, enhance immunotherapy and do not act as CD28 antagonists.

### SUMMARY OF THE INVENTION

The present invention provides single domain antibodies (sdAbs) that block CD28 cleavage. Dimeric agents comprising the sdAbs, methods of using the sdAbs and/or dimeric agents and pharmaceutical compositions and kits comprising the sdAbs and/or dimeric agents are also provided.

According to a first aspect, there is provided a single domain antibody (sdAb) comprising three CDRs wherein:
CDR1 comprises the amino acid sequence set forth in SEQ ID NO: 1 (INSMG), CDR2 comprises the amino acid sequence as set forth in SEQ ID NO: 2 (AINEKLLIYYADSVKG), CDR3 comprises the amino acid sequence as set forth in SEQ ID NO: 3 (DLYGSDYWD);
CDR1 comprises the amino acid sequence set forth in SEQ ID NO: 4 (INAMG), CDR2 comprises the amino acid sequence as set forth in SEQ ID NO: 5 (AISGGGDTYYADSVKG), CDR3 comprises the amino acid sequence as set forth in SEQ ID NO: 6 (DMIEQQWWY);
CDR1 comprises the amino acid sequence set forth in SEQ ID NO: 4 (INAMG), CDR2 comprises the amino acid sequence as set forth in SEQ ID NO: 5 (AISGGGDTYYADSVKG), CDR3 comprises the amino acid sequence as set forth in SEQ ID NO: 7 (DTHRGVYWY);
CDR1 comprises the amino acid sequence set forth in SEQ ID NO: 8 (IKTMA), CDR2 comprises the amino acid sequence as set forth in SEQ ID NO: 9 (AINYIKEIYYADSVKG), CDR3 comprises the amino acid sequence as set forth in SEQ ID NO: 10 (DVTKEDYWY);
CDR1 comprises the amino acid sequence set forth in SEQ ID NO: 11 (INSMA), CDR2 comprises the amino acid sequence as set forth in SEQ ID NO: 12 (AISNAREVYYADSVKG), CDR3 comprises the amino acid sequence as set forth in SEQ ID NO: 13 (DVYFQEYWY);
CDR1 comprises the amino acid sequence set forth in SEQ ID NO: 14 (INTMA), CDR2 comprises the amino acid sequence as set forth in SEQ ID NO: 15 (AINSISRTYYADSVKG), CDR3 comprises the amino acid sequence as set forth in SEQ ID NO: 10 (DVTKEDYWY);
CDR1 comprises the amino acid sequence set forth in SEQ ID NO: 8 (IKTMA), CDR2 comprises the amino acid sequence as set forth in SEQ ID NO: 16 (AIASDNRKYYADSVKG), CDR3 comprises the amino acid sequence as set forth in SEQ ID NO: 10 (DVTKEDYWY);
CDR1 comprises the amino acid sequence set forth in SEQ ID NO: 17 (IRTMA), CDR2 comprises the amino acid sequence as set forth in SEQ ID NO: 18 (AISSGREVYYADSVKG), CDR3 comprises the amino acid sequence as set forth in SEQ ID NO: 19 (DMYWQDYWW); or
CDR1 comprises the amino acid sequence set forth in SEQ ID NO: 1 (INSMG), CDR2 comprises the amino acid sequence as set forth in SEQ ID NO: 20 (AISDRSEKYYADSVKG), CDR3 comprises the amino acid sequence as set forth in SEQ ID NO: 21 (DHHHSDWWT).

According to some embodiments, the sdAb is a camelid or shark antibody.

According to some embodiments, the sdAb is a VHH antibody.

According to some embodiments, a sequence N-terminal to CDR1 consists of X1VQLVESGGGLVQX2GX3SLRLSCX4ASGSX5X6S (SEQ ID NO: X), wherein X1 is E or Q, X2 is A or P, X3 is E or G, X4 is A or K, X5 is I, L or T andX6 is A or F; a sequence between CDR1 and CDR2 consists of WYRQAPGX7X8X9EX10VX11 (SEQ ID NO: X), wherein X7 is S or K, X8 is Q or G, X9 is R or L, X10 is L or R, and X11 is one of: A, S, or T; a sequence between CDR2 and CDR3 consists of RFTX11SRDNX12KX13TX14YLQMNX15LX16X17X18DX19X20VYYCVV (SEQ ID NO: X), wherein X11 is I or V, X12 is A or S, X13 is T or N, X14 is V, M or L, X15 is S or N, X16 is R, K, or E, and X17 is P or A, X18 is E or R, X19 is T or A, X20 is A or G; and a sequence C-terminal to CDR3 consists of WGQGTX21VTVSS (SEQ ID NO: X), wherein X21 is an Q or L.

According to some embodiments, a sequence N-terminal to CDR1 consists of EVQLVESGGGLVQAGESLRLSCAASGSIAS (SEQ ID NO: 22), a sequence between CDR1 and CDR2 consists of WYRQAPGSQRELVX (SEQ ID NO: 48), a sequence between CDR2 and CDR3 consists of RFTISRDNAKTTVYLQMNSLRPEDTAVYYCVV (SEQ ID NO: 24) and a sequence C-terminal to CDR3 consists of WGQGTQVTVSS (SEQ ID NO: 25), wherein X is an A or T.

According to some embodiments, the sdAb comprises a sequence selected from a group consisting of:

a.
(SEQ ID NO: 26)
EVQLVESGGGLVQAGESLRLSCAASGSIASINSMGWYRQAPGSQREL
VAAINEKLLIYYADSVKGRFTISRDNAKTTVYLQMNSLRPEDTAVYY
CVVDLYGSDYWDWGQGTQVTVSS;
b.
(SEQ ID NO: 27)
EVQLVESGGGLVQAGESLRLSCAASGSIASINAMGWYRQAPGSQREL
VAAISGGGDTYYADSVKGRFTISRDNAKTTVYLQMNSLRPEDTAVY
YCVVDMIEQQWWYWGQGTQVTVSS;
c.
(SEQ ID NO: 28)
EVQLVESGGGLVQAGESLRLSCAASGSIASINAMGWYRQAPGSQREL
VAAISGGGDTYYADSVKGRFTISRDNAKTTVYLQMNSLRPEDTAVY
YCVVDTHRGVYWYWGQGTQVTVSS;
d.
(SEQ ID NO: 29)
EVQLVESGGGLVQAGESLRLSCAASGSIASIKTMAWYRQAPGSQREL
VAAINYIKEIYYADSVKGRFTISRDNAKTTVYLQMNSLRPEDTAVYY
CVVDVTKEDYWYWGQGTQVTVSS;
e.
(SEQ ID NO: 30)
EVQLVESGGGLVQAGESLRLSCAASGSIASINSMAWYRQAPGSQREL
VAAISNAREVYYADSVKGRFTISRDNAKTTVYLQMNSLRPEDTAVY
YCVVDVYFQEYWYWGQGTQVTVSS;
f.
(SEQ ID NO: 31)
EVQLVESGGGLVQAGESLRLSCAASGSIASINTMAWYRQAPGSQREL
VAAINSISRTYYADSVKGRFTISRDNAKTTVYLQMNSLRPEDTAVYY
CVVDVTKEDYWYWGQGTQVTVSS;
g.
(SEQ ID NO: 32)
EVQLVESGGGLVQAGESLRLSCAASGSIASIKTMAWYRQAPGSQREL
VTAIASDNRKYYADSVKGRFTISRDNAKTTVYLQMNSLRPEDTAVY
YCVVDVTKEDYWYWGQGTQVTVSS;
h.
(SEQ ID NO: 70)
EVQLVESGGGLVQPGGSLRLSCAASGSIASIKTMAWYRQAPGKQREL
VTAIASDNRKYYADSVKGRFTISRDNSKNTLYLQMNSLRAEDTAVY
YCVVDVTKEDYWYWGQGTLVTVSS;
i.
(SEQ ID NO: 71)
EVQLVESGGGLVQPGGSLRLSCKASGSIASIKTMAWYRQAPGKGLEL
VTAIASDNRKYYADSVKGRFTISRDNSKTTVYLQMNSLRAEDTAVY
YCVVDVTKEDYWYWGQGTLVTVSS;
j.
(SEQ ID NO: 72)
EVQLVESGGGLVQPGGSLRLSCAASGSTASIKTMAWYRQAPGKGLE
LVTAIASDNRKYYADSVKGRFTISRDNSKTTVYLQMNSLRAEDTAVY
YCVVDVTKEDYWYWGQGTLVTVSS;
k.
(SEQ ID NO: 73)
EVQLVESGGGLVQPGGSLRLSCKASGSTASIKTMAWYRQAPGKGLE
LVTAIASDNRKYYADSVKGRFTISRDNSKTTVYLQMNSLRAEDTAVY
YCVVDVTKEDYWYWGQGTLVTVSS;
l.
(SEQ ID NO: 74)
EVQLVESGGGLVQPGGSLRLSCAASGSIASIKTMAWYRQAPGKGREL
VTAIASDNRKYYADSVKGRFTISRDNSKTTVYLQMNSLRAEDTAVY
YCVVDVTKEDYWYWGQGTLVTVSS;
m.
(SEQ ID NO: 33)
EVQLVESGGGLVQAGESLRLSCAASGSIASIRTMAWYRQAPGSQREL
VAAISSGREVYYADSVKGRFTISRDNAKTTVYLQMNSLRPEDTAVYY
CVVDMYWQDYWWWGQGTQVTVSS;
n.
(SEQ ID NO: 75)
EVQLVESGGGLVQPGESLRLSCAASGSIASIRTMAWYRQAPGSQREL
VAAISSGREVYYADSVKGRFTISRDNAKTTVYLQMNSLRAEDTAVY
YCVVDMYWQDYWWWGQGTQVTVSS;
o.
(SEQ ID NO: 76)
EVQLVESGGGLVQPGGSLRLSCKASGSIASIRTMAWYRQAPGKGLEL
VAAISSGREVYYADSVKGRFTISRDNSKTTVYLQMNSLRAEDTAVYY
CVVDMYWQDYWWWGQGTLVTVSS;
p.
(SEQ ID NO: 77)
EVQLVESGGGLVQPGGSLRLSCKASGSTASIRTMAWYRQAPGKGLEL
VSAISSGREVYYADSVKGRFTISRDNSKTTVYLQMNSLRAEDTAVYY
CVVDMYWQDYWWWGQGTLVTVSS;
q.
(SEQ ID NO: 78)
EVQLVESGGGLVQPGGSLRLSCAASGSIASIRTMAWYRQAPGKGLEL
VSAISSGREVYYADSVKGRFTISRDNSKTTVYLQMNSLRAEDTAVYY
CVVDMYWQDYWWWGQGTLVTVSS;
or
r.
(SEQ ID NO: 34)
EVQLVESGGGLVQAGESLRLSCAASGSIASINSMGWYRQAPGSQREL
VAAISDRSEKYYADSVKGRFTISRDNAKTTVYLQMNSLRPEDTAVYY
CVVDHHHSDWWTWGQGTQVTVSS.

According to some embodiments, the sdAb is not a CD28 agonist.

According to some embodiments, the sdAb is not a CD28 antagonist.

According to some embodiments, the sdAb is a CD28 antagonist.

According to some embodiments, the agent neither degrades the mCD28 nor inhibits mCD28-mediated immune cell activation.

According to some embodiments, the agent binds within the stalk region of CD28.

According to some embodiments, the stalk region comprises the amino acid sequence GKHLCPSPLFPGPSKP (SEQ ID NO: 35) or KGKHLCPSPLFPGPS (SEQ ID NO: 36).

According to some embodiments, the stalk region consists of the amino acid sequence HVKGKHLCPSPLFPGPSKP (SEQ ID NO: 37).

According to some embodiments, the agent binds at a cleavage site for at least one protease.

According to some embodiments, the agent inhibits proteolytic cleavage by at least one protease.

According to some embodiments, the at least one protease is at least one metalloprotease.

According to some embodiments, the at least one metalloprotease is MMP-2, MMP-13, or a combination thereof.

According to another aspect, there is provided a dimeric agent comprising at least two membranal CD28 (mCD28) binding single domain antibodies (sdAbs), wherein a first mCD28 binding sdAb is linked to a second mCD28 binding sdAb by a linker.

According to some embodiments, the first sdAb, the second sdAb or both comprises a sequence selected from a group consisting of:

a.
(SEQ ID NO: 40)
EVQLVESGGGLVQAGESLRLSCAASGSIASINAMGWYRQAPGSQRELVA
AISGGGDTYYADSVKGRFTISRDNAKTTVYLQMNSLRPEDTAVYYCVVD
LYGSDYWDWGQGTQVTVSS;
b.
(SEQ ID NO: 95)
EVQLVESGGGLVQAGGSLRLSCAASGSLFSINAMAWYRQAPGKQRELVA
AITSSGSTNYANSVKGRFTVSRDNAKNTMYLQMNSLKPEDTAVYYCVVD
EYGSDYWIWGQGTQVTVSS;
and
c.
(SEQ ID NO: 96)
QVQLVESGGGLVQAGGSLRLSCAASGSIFSINAMGWYRQAPGKQRERVA
AITSGGSTNYADSVKGRFTISRDNAKNTVYLQMNNLEPRDAGVYYCVVD
LYGEDYWIWGQGTQVTVSS.

According to some embodiments, the dimeric agent comprises a sdAb of the invention.

According to some embodiments, the first sdAb and the second sdAb comprise the same sequence.

According to some embodiments, the first sdAb and the second sdAb comprise different sequences.

According to some embodiments, the dimeric agent inhibits proteolytic cleavage of the mCD28.

According to some embodiments, the first sdAb, the second sdAb or both when not part of a dimeric agent is a CD28 antagonist and wherein the dimeric agent is not a CD28 antagonist.

According to some embodiments, the dimeric agent comprises a first polypeptide comprising the first sdAb and a second polypeptide comprising the second sdAb and wherein the linker links the first polypeptide and the second polypeptide.

According to some embodiments, the first polypeptide comprises a first free cysteine amino acid outside of the first sdAb, the second polypeptide comprises a second free cysteine amino acid outside of the second sdAb and wherein the linker comprises a bond between the first and the second free cysteine amino acids.

According to some embodiments, the first free cysteine, the second free cysteine or both are C-terminal amino acids.

According to some embodiments, the first polypeptide comprises the first sdAb and a first dimerization domain, the second polypeptide comprises the second sdAb and a second dimerization domain and wherein the linker comprises the dimerization domains, a bond between the dimerization domains or both.

According to some embodiments, the first dimerization domain comprises a first immunoglobulin (Ig) hinge domain and the second dimerization domain comprises a second Ig hinge domain and wherein the linker comprises a disulfide bond between the first and second Ig hinge domains.

According to some embodiments, the first sdAb is N-terminal to the first dimerization domain, the second sdAb is N-terminal to the second dimerization domain or both.

According to some embodiments, the Ig hinge domain is a human Ig hinge domain comprising the amino acid sequence DKTHTCPPCPAPE (SEQ ID NO: 83) or ESKYGPPCPPCPAPEFEGG (SEQ ID NO: 85).

According to some embodiments, the first sdAb is separated from the first Ig hinge domain by an amino acid linker, the second sdAb is separated from the second Ig hinge domain by an amino acid linker, or both.

According to some embodiments, the amino acid linker is a flexible linker.

According to some embodiments, the amino acid linker comprises a sequence selected from (GGGGS)n, (GS)n, (GGS)n, (GSGGS)n, (EGGGS)n, (EGGS)n and a combination thereof, wherein n is an integer selected from 1, 2, 3, 4, 5, 6, 7, and 8.

According to some embodiments, the first dimerization domain, the second dimerization domain or both further comprise a CH2 domain of an Ig heavy chain.

According to some embodiments, the first dimerization domain, the second dimerization domain or both further comprises a CH3 domain of an Ig heavy chain.

According to some embodiments, the hinge domain is N-terminal to the CH2 domain and the CH2 domain is N-terminal to the CH3 domain.

According to some embodiments, the dimerization domain does not induce antibody-dependent cell-mediated cytotoxicity (ADCC) or complement-dependent cytotoxicity (CDC) or comprises at least one mutation that reduces ADCC or CDC.

According to some embodiments, the dimerization domain comprises

(SEQ ID NO: 39)
DKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALGAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK
or
(SEQ ID NO: 92)
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

According to some embodiments, the dimeric agent comprises

(SEQ ID NO: 93)
EVQLVESGGGLVQAGESLRLSCAASGSIASIKTMAWYRQAPGSQRELVT
AIASDNRKYYADSVKGRFTISRDNAKTTVYLQMNSLRPEDTAVYYCVVD
VTKEDYWYWGQGTQVTVSSGGGGSGGGGSGGGGSGGGGSGGGGSESKYG
PPCPPCPAPEFEGGPSVFLFPPKPKDTLMISRTPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSLGK.

According to some embodiments, the dimeric agent comprises

(SEQ ID NO: 94)
EVQLVESGGGLVQAGESLRLSCAASGSIASIRTMAWYRQAPGSQRELVA
AISSGREVYYADSVKGRFTISRDNAKTTVYLQMNSLRPEDTAVYYCVVD
MYWQDYWWWGQGTQVTVSSGGGGSGGGGSGGGGSGGGGSGGGGSESKYG
PPCPPCPAPEFEGGPSVFLFPPKPKDTLMISRTPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSLGK.

According to some embodiments, the dimeric agent does not inhibit or lowly inhibits binding of a ligand to CD28, wherein lowly inhibiting comprises less than 50% inhibition.

According to some embodiments, the ligand is CD86, CD80 or both.

According to some embodiments, the linker is a chemical linker.

According to some embodiments, the chemical linker comprises a biocompatible polymer.

According to some embodiments, the biocompatible polymer comprises polyethylene glycol (PEG).

According to some embodiments, the dimeric agent comprises a single polypeptide, wherein the single polypeptide comprises the first sdAb N-terminal to the second sdAb and separated by an amino acid linker of fewer than 13 amino acids, optionally wherein the dimeric agent is not a CD28 antagonist, inhibits ligand binding to CD28 by less than 50%, or both.

According to some embodiments, the dimeric agent comprises a single polypeptide, wherein the single polypeptide comprises the first sdAb N-terminal to the second sdAb and separated by an amino acid linker of equal to or greater than 10 amino acids.

According to some embodiments, the amino acid linker comprises a net neutral charge.

According to some embodiments, the amino acid linker comprises (a sequence selected from (GGGGS)n, (GS)n, (GGS)n, (GSGGS)n, and a combination thereof, wherein n is an integer selected from 1, 2, 3, 4, 5, 6, 7, and 8.

According to some embodiments, the amino acid linker comprises a net positive charge.

According to some embodiments, the amino acid linker comprises a sequence selected from (XGGGS)n, (XGGS)n, (GGGXS)n, and a combination thereof, wherein X is selected from K, R and H and n is an integer selected from 1, 2, 3, 4, 5, 6, 7 and 8.

According to some embodiments, X is K.

According to some embodiments, the amino acid linker comprises a net negative charge.

According to some embodiments, the amino acid linker comprises a sequence selected from (XGGGS)n, (XGGS)n, (GGGXS)n, and a combination thereof wherein X is selected from E and D and n is an integer selected from 1, 2, 3, 4, 5, 6, 7, and 8.

According to some embodiments, X is E.

According to some embodiments, the amino acid linker is a rigid linker.

According to some embodiments, the amino acid linker comprises

(SEQ ID NO: 97)
GGGGSAEAAAKEAAAKEAAAKAAAGSGGGGS.

According to some embodiments, the amino acid linker comprises at most 100 amino acids.

According to some embodiments, the dimeric agent is a CD28 antagonist.

According to some embodiments, the dimeric agent inhibits binding of a ligand to CD28, wherein inhibiting comprises at least 50% inhibition.

According to another aspect, there is provided a method of decreasing soluble CD28 (sCD28) levels in a subject in need thereof, the method comprising administering to the subject a sdAb of the invention or a dimeric agent of the invention, thereby decreasing sCD28.

According to another aspect, there is provided a method of treating and/or preventing cancer in a subject in need thereof, the method comprising administering to the subject a sdAb of the invention or a dimeric agent of the invention, thereby treating and/or preventing cancer.

According to another aspect, there is provided a method of improving PD-1 and/or PD-L1 based immunotherapy in a subject in need thereof, the method comprising administering to the subject a sdAb of the invention or a dimeric agent of the invention, thereby improving PD-1 and/or PD-L1 based immunotherapy.

According to some embodiments, the subject suffers from cancer.

According to some embodiments, the cancer is selected from melanoma, head and neck, non-small cell lung cancer, ovarian, kidney, gastric and colorectal.

According to some embodiments, the cancer comprises elevated levels of sCD28 or increasing levels of sCD28.

According to some embodiments, the method does not degrade mCD28.

According to some embodiments, the method does not decrease mCD28-mediated immune cell activation.

According to another aspect, there is provided a method of inhibiting ligand binding to mCD28, the method comprising contacting the mCD28 with a sdAb of the invention or a dimeric agent of the invention, thereby inhibiting ligand binding to mCD28.

According to another aspect, there is provided a method for suppressing an immune response in a subject in need thereof, the method comprising administering to the subject a sdAb of the invention or the dimeric agent of the invention, thereby suppressing an immune response.

According to some embodiments, the dimeric agent inhibits ligand binding to mCD28 thereby suppressing an immune response.

According to some embodiments, the ligand is CD86, CD80 or both.

According to some embodiments, the subject is afflicted with an autoimmune disease.

According to some embodiments, the autoimmune disease is selected from the group consisting of: lupus, rheumatoid arthritis, Crohn's disease, inflammatory bowel disease, Becht's disease, colitis, ulcerative colitis, diabetes, Graves' disease, and multiple sclerosis.

According to another aspect, there is provided a pharmaceutical composition comprising a sdAb of the invention or a dimeric agent of the invention and a pharmaceutical acceptable carrier, excipient or adjuvant.

According to some embodiments, the pharmaceutical composition of the invention, comprising a sdAb of the invention or a dimeric agent of the invention is for use in treating and/or preventing cancer or for improving PD-1 and/or PD-L1 based immunotherapy.

According to some embodiments, the pharmaceutical composition of the invention, comprising a sdAb of the invention or a dimeric agent of the invention is for use in inhibiting ligand binding to mCD28 or for suppressing an immune response.

According to another aspect, there is provided a kit comprising at least one sdAb of the invention or a dimeric agent of the invention.

According to some embodiments, the kit further comprises at least one of:
a. an anti-PD-1 and/or PD-L1 immunotherapy; and
b. a label stating the agent of the invention is for use with a PD-1 and/or PD-L1 based immunotherapy.

According to another aspect, there is provided a method of generating a dimeric agent that inhibits proteolytic cleavage of mCD28 on a surface of a cell, comprising at least one of:
a. i. obtaining an agent that binds to mCD28 on a cell surface and blocks cleavage of the mCD28 by a protease;
   ii. linking a first moiety of the agent to a second moiety of the agent via a linker to produce a dimeric agent;
   iii. testing an ability of the dimeric agent to block cleavage of mCD28 on a cell surface by a protease; and
   iv. selecting a dimeric agent that blocks cleavage of mCD28 on a cell surface; and
b. culturing a host cell comprising one or more vectors comprising one or more nucleic acid sequences encoding a dimeric agent, wherein the one or more nucleic acid sequences are that of a dimeric agent that was selected by:
   i. obtaining an agent that binds to mCD28 on a cell surface and blocks cleavage of the mCD28 by a protease;
   ii. linking a first moiety of the agent to a second moiety of the agent via a linker to produce a dimeric agent;
   iii. testing an ability of the dimeric agent to block cleavage of mCD28 on a cell surface by a protease; and
   iv. selecting an agent that blocks cleavage of mCD28 on a cell surface;
thereby generating an agent that inhibits proteolytic cleavage of mCD28 on a surface of a cell.

According to some embodiments, the obtained agent is a sdAb.

According to some embodiments, the obtaining an agent comprises:
a. immunizing a shark or camelid with an extracellular domain or fragment thereof of CD28 and collecting antibodies from the immunized organism or screening a library of agents for binding to an extracellular domain or fragment thereof of CD28 and selecting an agent that binds;
b. testing binding of the antibodies or agents that bind to mCD28 on a cell surface and selecting antibodies or agents that bind to mCD28 on a cell surface; and
c. testing cleavage of mCD28 on a cell in the presence of a protease and the selected antibodies or agents and further selected antibodies or agents that block cleavage of the mCD28 on a cell.

According to some embodiments, the extracellular domain or fragment thereof
a. is dimeric;
b. comprises a CD28 stalk domain; or
c. both.

According to some embodiments, the protease is selected from, MMP-2, and MMP-13.

According to some embodiments, the method further comprises assaying mCD28 downstream signaling in the presence of the obtained dimeric agent and selecting at least one dimeric agent that neither substantially agonizes nor substantially antagonizes mCD28 signaling.

According to some embodiments, the method further comprising assaying mCD28 downstream signaling in the presence of the obtained dimeric agent and selecting at least one dimeric agent that substantially antagonizes mCD28 signaling.

According to another aspect, there is provided a dimeric agent produced by a method of the invention.

According to another aspect, there is provided a pharmaceutical composition comprising a dimeric agent of the invention and a pharmaceutical acceptable carrier, excipient or adjuvant.

According to some embodiments, the pharmaceutical composition of the invention comprising a dimeric agent produced by a method of the invention is for use in treating and/or preventing cancer or for improving PD-1 and/or PD-L1 based immunotherapy.

According to some embodiments, the pharmaceutical composition of the invention comprising a dimeric agent produced by a method of the invention is for use in inhibiting ligand binding to mCD28 or for suppressing an immune response.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A line graph quantitation of soluble CD28 from SEB stimulated PBMC in the presence of parental clone (VHH #2A1) and two affinity matured variants.

FIG. 2: Bar graphs of IFN gamma secretion from isolated T cells in a mixed lymphocyte reaction (MLR, +mDC) in the presence of the parental VHH or two affinity matured variants. VHH #3C04=irrelevant VHH as negative control. VHH #12B07=known CD28 antagonist as positive control.

FIG. 3: Bar graphs of IL-2 secretion from isolated T cells in a mixed lymphocyte reaction (MLR, +mDC) in the presence of Fc dimeric agents comprising two copies of the parental VHH or two affinity matured variants. Human IgG4 was used as a negative control.

FIGS. 4A-4B: (4A) A line graph quantitation of the binding affinity of the VHH-hIgG4 constructs, 2A1-hFc, 9B03-hFc, and 12A09-hFc, to human CD28, using direct CD28 EIA. (4B) Histograms of 2A1-hFc, 9B03-hFc, and 12A09-hFc binding to human CD3 cells (black histogram). The background of anti-human IgG detection antibody is shown by the grey histogram.

FIGS. 5A-5B: Bar graphs demonstrating sCD28 production by peripheral blood mononuclear cells (PBMCs) in response to stimulation with staphylococcal enterotoxin B (SEB), with different concentrations of (5A) 12A09-25GS-hFc, and (5B) 9B03-25GS-hFc, compared to the corresponding isotype control and the parental 2A1-hFc clone.

FIGS. 6A-6B: Bar graphs of IL-2 secretion from CD3 cells stimulated with anti-CD3 antibody in the presence of (6A) the 12A9-25GS-huFc and (6B) the 9B3-25GS-huFc chimeric molecules. An agonistic anti-CD28 antibody, clone 28.2, was used as a positive control (PC) and an isotype control was used as a negative control.

FIG. 7: Bar charts showing the effect of flexible linker size on CD28 shedding inhibition activity of VHH 12A9. Two VHH concentrations, 100 nM (top) and 300 nM (bottom), were tested.

FIGS. 8A-8B: Line graphs demonstrating the tumor volume in MC-38 syngeneic tumor model, in response to: (8A) treatment with 12A09-hFc twice a week, starting 6 days prior MC38 cells inoculation (prevention model), and (8B) treatment with 9B03-hFc, twice a week, starting 6 days post MC38 cells inoculation (treatment model). Anti-PD1 (RMP1-14) was administered, twice a week, starting 6 days post MC38 inoculation. Human IgG4 and rat IgG2a served as negative controls. For 8A: rG2a, n=10; aPD1, n=10; VHH, n=10. For 8B: rG2a, n=10; aPD1, n=10; VHH, n=10. *=pVal of 0.014.

FIG. 9A-9B: Line graph quantitation of the binding affinity of (9A) five humanized 12A09-hIgG4 constructs, and (9B) one humanized 9B03-hIgG4 construct, to human CD28, using direct CD28 EIA. In both graphs the binding was compared to the parental variants (black line).

FIG. 10A-10B: Line graph quantitation of soluble CD28 from SEB stimulated PBMC (10A) in the presence of the 12A9-25GS-huFc chimera and five 12A09 humanized variants in the same chimera or (10B) in the presence of 9B3-25GS-huFc chimera and the humanized 9B3 variant VHH1 in the same chimera.

FIGS. 11A-11H: (11A-11B) Line graphs showing antigen binding by serial dilution of single chain tandem dimeric agents to recombinant human CD28-Fc fusion protein. Antigens were immobilized on Maxisorp ELISA plates. A dilution series of the dimeric agents was preformed, molar concentrations of different dimeric agents were calculated and normalized to compare the amount of active sites in each assay. Detection of bound antibody was done with anti-VHH-HRP and development with TMB. (11C-11H) Bar graphs of levels of soluble CD28 measured in culture media of PBMCs stimulated with SEB. The effect of different treatments of MMP inhibitor (TMI-1, 1 μM), negative control of irrelevant VHH or (11C) 5GS, (11D) 10GS, (11E) 20GS, (11F) 20K, (11G) 20E, or (11H) Hel20 dimeric agents at various concentrations (0.024 μM-3 μM) on the level of soluble CD28 is depicted. The levels of soluble human CD28 in the supernatant were quantified with standardized sandwich ELISA (R&D system).

FIGS. 12A-12B: Histograms of HEK293 cells over expressing human CD28 that were monitored by flow-cytometry for CD86-Fc (2 μg/mL) binding using secondary anti human Fc antibody conjugated to AlexaFlour 647. Monitoring was performed in the presence of (12A) neutral flexible linker or (12B) charged flexible linker or rigid linker containing single-chain dimeric agents. Light grey line-secondary antibody control. Black line-CD86-Fc positive control. Dark grey line-binding in the presence of the dimeric molecule.

FIGS. 13A-13I: (13A-13F) Bar graphs of IL-2 secretion from human isolated CD9 cells stimulated for 24-48 hours with HEK/scOKT3 serving as artificial antigen presenting cells expressing CD80 as ligand for CD28 co-stimulation, in the presence of an irrelevant negative control VHH, a positive control (PC) VHH known to block CD80 interaction with CD28 (PC) and the (13A) 5GS, (13B) 10GS, (13C) 20GS, (13D) 20K, (13E) 20E and (13F) Hel20 dimeric agents. (3G-I) Bar graphs of IFN gamma secretion from isolated T cells in an allogeneic mixed lymphocyte reaction (MLR, +mDC) in the presence of (13G) 5GS, (13H) 10GS, and (3I) 20GS dimeric agents.

FIGS. 14A-14J: (14A-14C) Line graphs showing antigen binding by serial dilution of (14A) single cysteine dimeric molecules, (14B) PEG linked dimeric molecules, and (14C) Fc linked dimeric molecules to recombinant human CD28-Fc fusion protein. (14D-I) Bar graphs of levels of soluble CD28 measured in culture media of PBMCs stimulated with SEB. The effect of different treatments of negative control of irrelevant VHH, monomeric 2A1 (3 μM) or (14D) 2A1-1C, (14E) 2A1-1C-bmP11, (14F) 2A1-Hinge, (14G) 2A1-hFC, (14H) 2A1-15GS-hFc, or (14I) 2A1-25GS-hFc dimeric agents at various concentrations (0.024 μM-3 μM) on the level of soluble CD28 is depicted. The levels of soluble human CD28 in the supernatant were quantified with standardized sandwich ELISA (R&D system). (14J) Summary scatter plot of the CD28-shedding inhibition produced by the various non-antagonistic dimeric molecules.

FIG. 15: Structure of the 2A1-bmp11-2A1 molecule that makes use of a linker with 11 PEG repeats.

FIGS. 16A-16B: Histograms of HEK293 cells over expressing human CD28 that were monitored by flow-cytometry for CD86-Fc (2 μg/mL) binding using secondary anti-human Fc antibody conjugated to AlexaFlour 647. Monitoring was performed in the presence of (16A) C-terminal linker or (16B) Ig-based linker containing dimeric agents.

FIGS. 17A-17F: Bar graphs of IL-2 secretion from human isolated CD3 cells stimulated for 24 hours with HEK/scOKT3 serving as artificial antigen presenting cells expressing CD80 as ligand for CD28 co-stimulation, in the presence of an irrelevant negative control VHH, a positive control VHH known to block CD86 and the (17A) 2A1-C, (17B) 2A1-1C-bmP11, (17C) 2A1-Hinge, (17D) 2A1-huFc, (17E) 2A1-15GS-huFc and (17F) 2A1-25GS-huFc dimeric agents.

FIGS. 18A-18F: (18A-18E) Bar graphs of IFN gamma secretion from isolated T cells in a mixed lymphocyte reaction (MLR, +mDC) in the presence of (18A) 2A1-C, (18B) 2A1-1C-bmP11, (18C) 2A1-huFc, (18D) 2A1-15GS-huFc and (18E) 2A1-25GS-huFc dimeric agents. (18F) Scatter plot of the total change in immune modulation in T cells.

### DETAILED DESCRIPTION OF THE INVENTION

The present invention, in some embodiments, provides single domain antibodies (sdAbs) that block CD28 cleavage. Dimeric agents comprising at least two membranal CD28 (mCD28) binding single domain antibodies (sdAbs) are also provided. Methods of treatment comprising administering the sdAbs and/or the dimeric agents as well as compositions and kits comprising the sdAbs and/or dimeric agents are also provided.

sdAbs

By a first aspect, there is provided a sdAb comprising three complementarity determining regions (CDRs), wherein CDR1 comprises the amino acid sequence set forth in SEQ ID NO: 1 (INSMG), CDR2 comprises the amino acid sequence as set forth in SEQ ID NO: 2 (AINEKLLIYYADSVKG), CDR3 comprises the amino acid sequence as set forth in

SEQ ID NO: 3
(DLYGSDYWD).

By another aspect, there is provided a sdAb comprising three complementarity determining regions (CDRs), wherein CDR1 comprises the amino acid sequence set forth in SEQ ID NO: 4 (INAMG), CDR2 comprises the amino acid sequence as set forth in SEQ ID NO: 5 (AISGGGDTYYADSVKG), CDR3 comprises the amino acid sequence as set forth in SEQ ID NO: 6 (DMIEQQWWY).

By another aspect, there is provided a sdAb comprising three complementarity determining regions (CDRs), wherein CDR1 comprises the amino acid sequence set forth in SEQ ID NO: 4 (INAMG), CDR2 comprises the amino acid sequence as set forth in SEQ ID NO: 5 (AISGGGDTYYADSVKG), CDR3 comprises the amino acid sequence as set forth in SEQ ID NO: 7 (DTHRGVYWY).

By another aspect, there is provided a sdAb comprising three complementarity determining regions (CDRs), wherein CDR1 comprises the amino acid sequence set forth in SEQ ID NO: 8 (IKTMA), CDR2 comprises the amino acid sequence as set forth in SEQ ID NO: 9 (AINYIKEIYYADSVKG), CDR3 comprises the amino acid sequence as set forth in SEQ ID NO: 10 (DVTKEDYWY).

SEQ ID NO: 10
(DVTKEDYWY).

By another aspect, there is provided a sdAb comprising three complementarity determining regions (CDRs), wherein CDR1 comprises the amino acid sequence set forth in SEQ ID NO: 11 (INSMA), CDR2 comprises the amino acid sequence as set forth in SEQ ID NO: 12 (AISNAREVYYADSVKG), CDR3 comprises the amino acid sequence as set forth in SEQ ID NO: 13 (DVYFQEYWY).

By another aspect, there is provided a sdAb comprising three complementarity determining regions (CDRs), wherein CDR1 comprises the amino acid sequence set forth in SEQ ID NO: 14 (INTMA), CDR2 comprises the amino acid sequence as set forth in SEQ ID NO: 15 (AINSISRTYYADSVKG), CDR3 comprises the amino acid sequence as set forth in SEQ ID NO: 10 (DVTKEDYWY).

By another aspect, there is provided a sdAb comprising three complementarity determining regions (CDRs), wherein CDR1 comprises the amino acid sequence set forth in SEQ ID NO: 8 (IKTMA), CDR2 comprises the amino acid sequence as set forth in SEQ ID NO: 16 (AIASDNRKYYADSVKG), CDR3 comprises the amino acid sequence as set forth in SEQ ID NO: 10 (DVTKEDYWY).

By another aspect, there is provided a sdAb comprising three complementarity determining regions (CDRs), wherein CDR1 comprises the amino acid sequence set forth in SEQ ID NO: 17 (IRTMA), CDR2 comprises the amino acid sequence as set forth in SEQ ID NO: 18 (AISSGREVYYADSVKG), CDR3 comprises the amino acid sequence as set forth in SEQ ID NO: 19 (DMYWQDYWW).

By another aspect, there is provided a sdAb comprising three complementarity determining regions (CDRs), wherein CDR1 comprises the amino acid sequence set forth in SEQ ID NO: 1 (INSMG), CDR2 comprises the amino acid sequence as set forth in SEQ ID NO: 20 (AISDRSEKYYADSVKG), CDR3 comprises the amino acid sequence as set forth in SEQ ID NO: 21 (DHHHSDWWT).

As used herein, the terms “single domain antibody”, “nanobody”, “DARPin” and “VHH antibody” are synonymous and used interchangeably and refer to an antibody fragment consisting of a single monomeric variable domain. SdAbs are capable of selectively binding to specific antigens just as antibodies do. However, they have a molecular weight of only 12-15 kDa and thus are much smaller than full antibodies, Fab fragments or single-chain antibodies. Due to its small size and that antigen-binding relies on only 3 CDRs, the binding machinery of a single domain antibody, specifically a VHH, is of a convex shape and binds its epitope from only one side and is more thus suited to bind epitopes that are characterized by limited solvent exposure, such as found in protein clefts like the stalk region of membrane anchored CD28. In some embodiments, the sdAb is a camelid antibody. In some embodiments, a camelid is a camel, an alpaca or a llama. In some embodiments, the camelid is a camel. In some embodiments, the camelid is an alpaca. In some embodiments, the camelid is a llama. In some embodiments, the sdAb is a shark antibody. In some embodiments, the sdAb is a first sdAb in a molecule. In some embodiments, the sdAb is a second sdAb in a molecule. In some embodiments, the molecule is a dimeric molecule of the invention.

Also, as already indicated herein, the amino acid residues of a Nanobody are numbered according to the general numbering for VHs given by Kabat et al. (“Sequence of proteins of immunological interest”, US Public Health Services, NIH Bethesda, Md., Publication No. 91), as applied to VHH domains from Camelids in the article of Riechmann and Muyldermans, J. Immunol, Methods 2000 Jun. 23; 240 (1-2): 185-195; or referred to herein. According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. In this respect, it should be noted that as is well known in the art for VH domains and for VHH domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.

Alternative methods for numbering the amino acid residues of VH domains, which methods can also be applied in an analogous manner to VHH domains from Camelids and to Nanobodies, are the method described by Chothia et al. (Nature 342, 877-883 (1989)), the so-called “AbM definition” and the so-called “contact definition”. However, in the present description, aspects and figures, the numbering according to Kabat as applied to VHH domains by Riechmann and Muyldermans will be followed, unless indicated otherwise.

In some embodiments, the sdAb binds to CD28. In some embodiments, the agent binds to CD28. In some embodiments, the CD28 is mammalian CD28. In some embodiments the CD28 is human CD28. In some embodiments, the human CD28 comprises or consists of the amino acid sequence: MLRLLLALNLFPSIQVTGNKILVKQSPMLVAYDNAVNLSCKYSYNLFSREFRASLH KGLDSAVEVCVVYGNYSQQLQVYSKTGFNCDGKLGNESVTFYLQNLYVNQTDIY FCKIEVMYPPPYLDNEKSNGTIIHVKGKHLCPSPLFPGPSKPFWVLVVVGGVLACY SLLVTVAFIIFWVRSKRSRLLHSDYMNMTPRRPGPTRKHYQPYAPPRDFAAYRS (SEQ ID NO: 42). In some embodiments, mature CD28 lacks a signal peptide and comprises the sequence: NKILVKQSPMLVAYDNAVNLSCKYSYNLFSREFRASLHKGLDSAVEVCVVYGNY SQQLQVYSKTGFNCDGKLGNESVTFYLQNLYVNQTDIYFCKIEVMYPPPYLDNEK SNGTIIHVKGKHLCPSPLFPGPSKPFWVLVVVGGVLACYSLLVTVAFIIFWVRSKRS RLLHSDYMNMTPRRPGPTRKHYQPYAPPRDFAAYRS (SEQ ID NO: 43). In some embodiments, the CD28 signal peptide comprises or consists of MLRLLLALNLFPSIQVTG (SEQ ID NO: 41).

In some embodiments, the DNA coding sequence that codes for full length human CD28 comprises the sequence:

(SEQ ID NO: 44)
ATGCTCAGGCTGCTCTTGGCTCTCAACTTATTCCCTTCAATTCAAGTAA
CAGGAAACAAGATTTTGGTGAAGCAGTCGCCCATGCTTGTAGCGTACGA
CAATGCGGTCAACCTTAGCTGCAAGTATTCCTACAATCTCTTCTCAAGG
GAGTTCCGGGCATCCCTTCACAAAGGACTGGATAGTGCTGTGGAAGTCT
GTGTTGTATATGGGAATTACTCCCAGCAGCTTCAGGTTTACTCAAAAAC
GGGGTTCAACTGTGATGGGAAATTGGGCAATGAATCAGTGACATTCTAC
CTCCAGAATTTGTATGTTAACCAAACAGATATTTACTTCTGCAAAATTG
AAGTTATGTATCCTCCTCCTTACCTAGACAATGAGAAGAGCAATGGAAC
CATTATCCATGTGAAAGGGAAACACCTTTGTCCAAGTCCCCTATTTCCC
GGACCTTCTAAGCCCTTTTGGGTGCTGGTGGTGGTTGGTGGAGTCCTGG
CTTGCTATAGCTTGCTAGTAACAGTGGCCTTTATTATTTTCTGGGTGAG
GAGTAAGAGGAGCAGGCTCCTGCACAGTGACTACATGAACATGACTCCC
CGCCGCCCCGGGCCCACCCGCAAGCATTACCAGCCCTATGCCCCACCAC
GCGACTTCGCAGCCTATCGCTCCTGA.

In some embodiments, the CD28 is membranal CD28 (mCD28). In some embodiments, membranal CD28 is membrane CD28. In some embodiments, the mCD28 is on a cell surface. In some embodiments, the mCD28 is in a membrane.

In some embodiments, CD28 is extracellular CD28. In some embodiments, CD28 is the extracellular domain (ECD) of CD28. In some embodiments, the ECD of CD28 comprises MLRLLLALNLFPSIQVTGNKILVKQSPMLVAYDNAVNLSCKYSYNLFSREFRASLH KGLDSAVEVCVVYGNYSQQLQVYSKTGFNCDGKLGNESVTFYLQNLYVNQTDIY FCKIEVMYPPPYLDNEKSNGTIIHVKGKHLCPSPLFPGPSKP (SEQ ID NO: 45). In some embodiments, the ECD of CD28 consists of SEQ ID NO: 45. In some embodiments, the ECD comprises NKILVKQSPMLVAYDNAVNLSCKYSYNLFSREFRASLHKGLDSAVEVCVVYGNY SQQLQVYSKTGFNCDGKLGNESVTFYLQNLYVNQTDIYFCKIEVMYPPPYLDNEK SNGTIIHVKGKHLCPSPLFPGPSKP (SEQ ID NO: 46). In some embodiments, the ECD consists of SEQ ID NO: 46. In some embodiments, the ECD is dimeric. In some embodiments, the ECD comprises the stalk domain.

In some embodiments, CD28 is the stalk domain of CD28. In some embodiments, a first CD28 binding sdAb binds the stalk domain. In some embodiments, a second CD28 binding sdAb binds the stalk domain. In some embodiments, both the first and second CD28 binding sdAbs bind the stalk domain. In some embodiments, the stalk domain is the stalk region. In some embodiments, the stalk region comprises the amino acid sequence GKHLCPSPLFPGPSKP (SEQ ID NO: 35). In some embodiments, the stalk region comprises the amino acid sequence KGKHLCPSPLFPGPS (SEQ ID NO: 36). In some embodiments, the stalk region comprises the amino acid sequence HVKGKHLCPSPLFPGPSKP (SEQ ID NO: 37). In some embodiments, the stalk region consists of SEQ ID NO: 35. In some embodiments, the stalk region consists of SEQ ID NO: 36. In some embodiments, the stalk region consists of SEQ ID NO: 37.

In some embodiments, the sdAb inhibits proteolytic cleavage of CD28. As used herein, “inhibiting proteolytic cleavage” refers to any reduction in proteolytic cleavage of mCD28. In some embodiments, the inhibition is a reduction in cleavage of at least 5, 10, 15, 20, 25, 30, 35, 40, 45, 50, 55, 60, 65, 70, 75, 80, 85, 90, 95, 97, 99 or 100%. Each possibility represents a separate embodiment of the invention. In some embodiments, inhibiting proteolytic cleavage maintains levels of mCD28 on immune cells. In some embodiments, inhibiting proteolytic cleavage increases levels of mCD28 on immune cells. In some embodiments, inhibiting proteolytic cleavage maintains levels of mCD28 adequate for immune stimulation.

In some embodiments, the reduction in proteolytic cleavage is reduction in cleavage by at least one protease. In some embodiments, the reduction in proteolytic cleavage is reduction in cleavage by at least one metalloprotease. In some embodiments, the metalloprotease is MMP-2, ADAM10, ADAM17 or a combination thereof. In some embodiments, the metalloprotease is MMP-2, ADAM10, ADAM17, MMP-13 or a combination thereof. In some embodiments, the metalloprotease is MMP-2. In some embodiments, the metalloprotease is MMP-2 or MMP-13. In some embodiments, the metalloprotease is MMP-2. In some embodiments, the metalloprotease is MMP-2, MMP-13 or a combination thereof.

In some embodiments, the sdAb inhibits proteolytic cleavage by at least one protease. In some embodiments, the protease is a metalloprotease. In some embodiments, the protease is a matrix metalloprotease. In some embodiments, the protease is a serine protease. In some embodiments, the protease is a cysteine protease. In some embodiments, the protease is a threonine protease. In some embodiments, the protease is a serine, cysteine or threonine protease. In some embodiments, the protease is an aspartic protease. In some embodiments, the protease is a glutamic protease. In some embodiments, the protease is selected from an aspartic, a glutamic, a serine, a cysteine and a threonine protease. In some embodiments, the protease is an asparagine peptide lyases. In some embodiments, the protease is a sheddase. In some embodiments, the metalloprotease is an exopeptidase. In some embodiments, the metalloprotease is an endopeptidase. In some embodiments, the metalloprotease is an exopeptidase or endopeptidase. In some embodiments, the metalloprotease is zinc catalyzed. In some embodiments, the metalloprotease is cobalt catalyzed. In some embodiments, the metalloprotease is matrix metalloproteinase-2 (MMP-2). In some embodiments, the metalloprotease is matrix metalloproteinase-13 (MMP-13). In some embodiments, the metalloprotease is ADAM10. In some embodiments, the metalloprotease is ADAM17. In some embodiments, the metalloprotease is ADAM10, MMP-2, and/or ADAM17. In some embodiments, the metalloprotease is ADAM10, MMP-2, MMP-13 and/or ADAM17. In some embodiments, the metalloprotease is MMP-2, ADAM10, ADAM17 or a combination thereof. In some embodiments, the metalloprotease is MMP-2, MMP-13, ADAM10, ADAM17 or a combination thereof.

In some embodiments, the sdAb binds to a cleavage site. In some embodiments, the cleavage site is within the stalk region. In some embodiments, the cleavage site is a cleavage motif. In some embodiments, the MMP-2 cleavage motif is PXX/X, wherein the last X is a hydrophobic residue. In some embodiments, the PXX/X motif in CD28 is PSP/L. In some embodiments, the protease cleavage site is amino acids 142-145 (PSPL) of SEQ ID NO: 42. In some embodiments, the protease cleavage site is amino acids 127-130 (PSPL) of SEQ ID NO: 43. In some embodiments, the protease cleavage site is amino acids 9-12 (PSPL) of SEQ ID NO: 37. In some embodiments, the agent blocks accesses of a protease to a cleavage site. In some embodiments, the agent binds to PSPL in a stalk domain of mCD28.

In some embodiments, the cleavage site is before a leucine. In some embodiments, the cleavage site is before a valine. In some embodiments, the cleavage site is before an aromatic amino acid. In some embodiments, the cleavage site is before a leucine, valine and/or aromatic amino acid. In some embodiments, the aromatic amino acid is selected from phenylalanine, tryptophan, tyrosine and histidine. In some embodiments, the cleavage site is before any one of histidine 134, valine 135, histidine 139, leucine 140, leucine 145, and phenylalanine 146 of SEQ ID NO: 29. In some embodiments, the cleavage site is before histidine 134, valine 135, histidine 139, leucine 140, leucine 145, or phenylalanine 146 of SEQ ID NO: 42. Each possibility represents a separate embodiment of the invention. In some embodiments, the cleavage site is before leucine 145 of SEQ ID NO: 42. In some embodiments, the cleavage site is before leucine 127 of SEQ ID NO: 43.

In some embodiments, the sdAb is not an antagonist. In some embodiments, the sdAb is not an antagonist of CD28. In some embodiments, an antagonist is a substantial antagonist. In some embodiments, an antagonist is a direct antagonist.

The term “antagonist” generally refers to a molecule, compound or agent that binds to a receptor at the same site as an agonist or another site, does not activate the receptor and does one or more of the following: interferes with or blocks activation of the receptor by a natural ligand, and interferes with or blocks activation of the receptor by a receptor agonist. In some embodiments, the sdAb binds to CD28 and blocks activation of the receptor. In some embodiments, the agent, and/or the sdAb does not block activation by CD86. In some embodiments, the sdAb does block activation by CD86. In some embodiments, the sdAb does block binding of a ligand to CD28. In some embodiments, the sdAb does not inhibit binding of a ligand to CD28. In some embodiments, the sdAb does inhibit binding of a ligand to CD28. In some embodiments, inhibit is substantially inhibit. In some embodiments, substantial is significantly. In some embodiments, substantial antagonism is more than low inhibition. In some embodiments, an agent that is not a substantial antagonist does not inhibit or lowly inhibits. In some embodiments, the sdAb lowly inhibits binding of a ligand to CD28. In some embodiments, lowly inhibits comprises less than 50, 45, 40, 35, 30, 25, 20, 15, 10, 7, or 5% inhibition. Each possibility represents a separate embodiment of the invention. In some embodiments, lowly inhibits comprises less than 50% inhibition. In some embodiments, lowly inhibits comprises less than 35% inhibition. In some embodiments, lowly inhibits comprises less than 20% inhibition. In some embodiments, the CD28 ligand is selected from: CD80, CD86 and ICOSL. In some embodiments, the CD28 ligand is CD86. In some embodiments, the CD28 ligand is CD80. In some embodiments, the CD28 ligand is ICOSL. In some embodiments, CD86 is CD86-Fc. In some embodiments, CD80 is CD80-Fc.

In some embodiments, the sdAb is not an agonist of CD28 In some embodiments, an agonist is a direct agonist. The term “agonist” generally refers to a molecule, compound or agent that binds to a receptor and activates, fully or partially, the receptor. In some embodiments, the agonist binds at the same site as the natural ligand. In some embodiments, the agonist binds at an allosteric site different from the binding site of the natural ligand.

As used herein, a “direct agonist/antagonist” refers to a molecule that binds to a receptor (mCD28) and by binding increases/decreases signaling by that molecule. In the case of mCD28 an agonist would bind mCD28 and by binding increase mCD28 signaling in the cell. In some embodiments, the agonist increases T cell activation. In some embodiments, the agonist increases T cell proliferation. In some embodiments, the agonist increases pro-inflammatory cytokine secretion. Pro-inflammatory cytokines are well known in the art and are known to be secreted by activated T cells. Examples of pro-inflammatory cytokines include, but are not limited to, TNFα, IFNγ, TL-1B, IL-2, and IL-6. In some embodiments, the pro-inflammatory cytokine is IFNγ. In some embodiments, the pro-inflammatory cytokine is IL-2. In the case of mCD28 an antagonist would bind mCD28 and by binding decrease mCD28 signaling in the cell. In some embodiments, the antagonist decreases T cell activation, decreases T cell proliferation and/or decreases pro-inflammatory cytokine secretion. A molecule that effects a receptor's signaling by contacting its ligand, contacting an inhibitor, contacting a co-receptor or contacting any molecule other than the receptor in question in order to modify receptor signaling is not considered a direct agonist/antagonist. In some embodiments, a sdAb of the invention decreases production of soluble CD28 (sCD28) (by inhibiting cleavage of mCD28). In some embodiments, a agent of the invention decreases production of soluble CD28 (sCD28) (by inhibiting cleavage of mCD28). sCD28 can act as a decoy by binding CD28 ligands as thus can be antagonistic to mCD28. Its removal allows for increased signaling through mCD28 on cells. Though the result is increased mCD28 signaling the agent is not a mCD28 agonist or direct agonist as its binding to mCD28 does not increase the receptors signaling.

In some embodiments, the sdAb does not bind the ligand binding domain of mCD28. In some embodiments, the sdAb does not obscure or block access to the ligand binding domain. In some embodiments, the sdAb does not bind, obscure or block access to the IgV domain of sCD28. In some embodiments, the IgV domain is the ligand binding domain. In some embodiments, the ligand binding domain comprises amino acids 28-137 of SEQ ID NO: 42. In some embodiments, the ligand binding domain comprises or consists of the amino acid sequence

(SEQ ID NO: 47)
MLVAYDNAVNLSCKYSYNLFSREFRASLHKGLDSAVEVCVVYGNYSQQL
QVYSKTGFNCDGKLGNESVTFYLQNLYVNQTDIYFCKIEVMYPPPYLDN
EKSNGTIIHVKG.

In some embodiments, the sdAb comprises the sequence X₁VQLVESGGGLVQX₂GX₃SLRLSCX₄ASGSX₅X₆S (SEQ ID NO: 79) N-terminal to CDR1 wherein X₁ is E or Q, X₂ is A or P, X₃ is E or G, X₄ is A or K, X₅ is I, L or T and X₆ is A or F. In some embodiments, a sequence N-terminal to CDR1 consists of SEQ ID NO: 79. In some embodiments, the sdAb comprises the sequence EVQLVESGGGLVQAGESLRLSCAASGSIAS (SEQ ID NO: 22) N-terminal to CDR1. In some embodiments, SEQ ID NO: 79 is SEQ ID NO: 22. In some embodiments, a sequence N-terminal to CDR1 consists of SEQ ID NO: 22. In some embodiments, the sdAb comprises the sequence WYRQAPGX₇X₈X₉EX₁₀VX₁₁ (SEQ ID NO: 80) between CDR1 and CDR2, wherein X₇ is S or K, X₈ is Q or G, X₉ is R or L, X₁₀ is L or R, and X₁₁ is one of: A, S, or T. In some embodiments, a sequence between CDR1 and CDR2 consists of SEQ ID NO: 79. In some embodiments, the sdAb comprises the sequence WYRQAPGSQRELVX (SEQ ID NO: 48) between CDR1 and CDR2, wherein X is A or T. In some embodiments, a sequence between CDR1 and CDR2 consists of SEQ ID NO: 48. In some embodiments, SEQ ID NO: 80 is SEQ ID NO: 48. In some embodiments, SEQ ID NO: 48 is WYRQAPGSQRELVA (SEQ ID NO: 23). In some embodiments, SEQ ID NO: 48 is WYRQAPGSQRELVT (SEQ ID NO: 49). In some embodiments, the sdAb comprises the sequence RFTX₁₁SRDNX₁₂KX₁₃TX₁₄YLQMNX₁₅LX₁₆X₁₇X₁₈DX₁₉X₂₀VYYCVV (SEQ ID NO: 81) between CDR2 and CDR3, wherein X₁₁ is I or V, X₁₂ is A or S, X₁₃ is T or N, X₁₄ is V, M or L, X₁₅ is S or N, X₁₆ is R, K, or E, X₁₇ is P or A, X₁₈ is E or R, X₁₉ is T or A, X₂₀ is A or G. In some embodiments, a sequence between CDR2 and CDR3 consists of SEQ ID NO: 81. In some embodiments, the sdAb comprises the sequence RFTISRDNAKTTVYLQMNSLRPEDTAVYYCVV (SEQ ID NO: 24) between CDR2 and CDR3. In some embodiments, SEQ ID NO: 81 is SEQ ID NO: 24. In some embodiments, a sequence between CDR2 and CDR3 consists of SEQ ID NO: 24. In some embodiments, the sdAb comprises the sequence WGQGTX₂₁VTVSS (SEQ ID NO: 82) C-terminal to CDR3, wherein X₂₁ is an Q or L. In some embodiments, a sequence C-terminal to CDR3 consists of SEQ ID NO: 82. In some embodiments, the sdAb comprises the sequence WGQGTQVTVSS (SEQ ID NO: 25) C-terminal to CDR3. In some embodiments, SEQ ID NO: 82 is SEQ ID NO: 25. In some embodiments, a sequence C-terminal to CDR3 consists of SEQ ID NO: 25.

In some embodiments, the sdAb comprises the amino acid sequence EVQLVESGGGLVQAGESLRLSCAASGSIASINSMGWYRQAPGSQRELVAAINEKLL IYYADSVKGRFTISRDNAKTTVYLQMNSLRPEDTAVYYCVVDLYGSDYWDWGQ GTQVTVSS (SEQ ID NO: 26). In some embodiments, the sdAb consists of SEQ ID NO: 26. In some embodiments, SEQ ID NO: 26 is the amino acid sequence of VHH 5A3. In some embodiments, the sdAb comprises or consists of a sequence with at least 70, 75, 80, 85, 90, 93, 95, 97, or 99% homology to SEQ ID NO: 26. Each possibility represents a separate embodiment of the invention. In some embodiments, the sequence with homolog comprises the CDRs of SEQ ID NO: 26.

In some embodiments, the sdAb comprises the amino acid sequence EVQLVESGGGLVQAGESLRLSCAASGSIASINAMGWYRQAPGSQRELVAAISGGG DTYYADSVKGRFTISRDNAKTTVYLQMNSLRPEDTAVYYCVVDMIEQQWWYWG QGTQVTVSS (SEQ ID NO: 27). In some embodiments, the sdAb consists of SEQ ID NO: 27. In some embodiments, SEQ ID NO: 27 is the amino acid sequence of VHH 6B3. In some embodiments, the sdAb comprises or consists of a sequence with at least 70, 75, 80, 85, 90, 93, 95, 97, or 99% homology to SEQ ID NO: 27. Each possibility represents a separate embodiment of the invention. In some embodiments, the sequence with homolog comprises the CDRs of SEQ ID NO: 27.

In some embodiments, the sdAb comprises the amino acid sequence EVQLVESGGGLVQAGESLRLSCAASGSIASINAMGWYRQAPGSQRELVAAISGGG DTYYADSVKGRFTISRDNAKTTVYLQMNSLRPEDTAVYYCVVDTHRGVYWYWG QGTQVTVSS (SEQ ID NO: 28). In some embodiments, the sdAb consists of SEQ ID NO: 28. In some embodiments, SEQ ID NO: 28 is the amino acid sequence of VHH 6B10. In some embodiments, the sdAb comprises or consists of a sequence with at least 70, 75, 80, 85, 90, 93, 95, 97, or 99% homology to SEQ ID NO: 28. Each possibility represents a separate embodiment of the invention. In some embodiments, the sequence with homolog comprises the CDRs of SEQ ID NO: 28.

In some embodiments, the sdAb comprises the amino acid sequence EVQLVESGGGLVQAGESLRLSCAASGSIASIKTMAWYRQAPGSQRELVAAINYIKE IYYADSVKGRFTISRDNAKTTVYLQMNSLRPEDTAVYYCVVDVTKEDYWYWGQ GTQVTVSS (SEQ ID NO: 29). In some embodiments, the sdAb consists of SEQ ID NO: 29. In some embodiments, SEQ ID NO: 29 is the amino acid sequence of VHH 10E1. In some embodiments, the sdAb comprises or consists of a sequence with at least 70, 75, 80, 85, 90, 93, 95, 97, or 99% homology to SEQ ID NO: 29. Each possibility represents a separate embodiment of the invention. In some embodiments, the sequence with homolog comprises the CDRs of SEQ ID NO: 29.

In some embodiments, the sdAb comprises the amino acid sequence EVQLVESGGGLVQAGESLRLSCAASGSIASINSMAWYRQAPGSQRELVAAISNARE VYYADSVKGRFTISRDNAKTTVYLQMNSLRPEDTAVYYCVVDVYFQEYWYWGQ GTQVTVSS (SEQ ID NO: 30). In some embodiments, the sdAb consists of SEQ ID NO: 30. In some embodiments, SEQ ID NO: 30 is the amino acid sequence of VHH 11E11. In some embodiments, the sdAb comprises or consists of a sequence with at least 70, 75, 80, 85, 90, 93, 95, 97, or 99% homology to SEQ ID NO: 30. Each possibility represents a separate embodiment of the invention. In some embodiments, the sequence with homolog comprises the CDRs of SEQ ID NO: 30.

In some embodiments, the sdAb comprises the amino acid sequence EVQLVESGGGLVQAGESLRLSCAASGSIASINTMAWYRQAPGSQRELVAAINSISR TYYADSVKGRFTISRDNAKTTVYLQMNSLRPEDTAVYYCVVDVTKEDYWYWGQ GTQVTVSS (SEQ ID NO: 31). In some embodiments, the sdAb consists of SEQ ID NO: 31. In some embodiments, SEQ ID NO: 31 is the amino acid sequence of VHH 11G11. In some embodiments, the sdAb comprises or consists of a sequence with at least 70, 75, 80, 85, 90, 93, 95, 97, or 99% homology to SEQ ID NO: 31. Each possibility represents a separate embodiment of the invention. In some embodiments, the sequence with homolog comprises the CDRs of SEQ ID NO: 31.

In some embodiments, the sdAb comprises the amino acid sequence EVQLVESGGGLVQAGESLRLSCAASGSIASIKTMAWYRQAPGSQRELVTAIASDN RKYYADSVKGRFTISRDNAKTTVYLQMNSLRPEDTAVYYCVVDVTKEDYWYWG QGTQVTVSS (SEQ ID NO: 32). In some embodiments, the sdAb consists of SEQ ID NO: 32. In some embodiments, SEQ ID NO: 32 is the amino acid sequence of VHH 12A9. In some embodiments, the sdAb comprises or consists of a sequence with at least 70, 75, 80, 85, 90, 93, 95, 97, or 99% homology to SEQ ID NO: 32. Each possibility represents a separate embodiment of the invention. In some embodiments, the sequence with homolog comprises the CDRs of SEQ ID NO: 32.

In some embodiments, the first sdAb, the second sdAb or both comprises the amino acid sequence EVQLVESGGGLVQPGGSLRLSCAASGSIASIKTMAWYRQAPGKQRELVTAIASDN RKYYADSVKGRFTISRDNSKNTLYLQMNSLRAEDTAVYYCVVDVTKEDYWYWG QGTLVTVSS (SEQ ID NO: 70). In some embodiments, SEQ ID NO: 70 is the amino acid sequence of 12A09_VHH4. In some embodiments, the first sdAb, the second sdAb or both consists of SEQ ID NO: 70. In some embodiments, the first sdAb, the second sdAb or both comprises or consists of a sequence with at least 70, 75, 80, 85, 90, 93, 95, 97, or 99% homology to SEQ ID NO: 70. Each possibility represents a separate embodiment of the invention. In some embodiments, the sequence with homolog comprises the CDRs of SEQ ID NO: 70.

In some embodiments, the first sdAb, the second sdAb or both comprises the amino acid sequence EVQLVESGGGLVQPGGSLRLSCKASGSIASIKTMAWYRQAPGKGLELVTAIASDN RKYYADSVKGRFTISRDNSKTTVYLQMNSLRAEDTAVYYCVVDVTKEDYWYWG QGTLVTVSS (SEQ ID NO: 71). In some embodiments, SEQ ID NO: 71 is the amino acid sequence of 12A9_VHH12. In some embodiments, the first sdAb, the second sdAb or both consists of SEQ ID NO: 71. In some embodiments, the first sdAb, the second sdAb or both comprises or consists of a sequence with at least 70, 75, 80, 85, 90, 93, 95, 97, or 99% homology to SEQ ID NO: 71. Each possibility represents a separate embodiment of the invention. In some embodiments, the sequence with homolog comprises the CDRs of SEQ ID NO: 71.

In some embodiments, the first sdAb, the second sdAb or both comprises the amino acid sequence EVQLVESGGGLVQPGGSLRLSCAASGSTASIKTMAWYRQAPGKGLELVTAIASDN RKYYADSVKGRFTISRDNSKTTVYLQMNSLRAEDTAVYYCVVDVTKEDYWYWG QGTLVTVSS (SEQ ID NO: 72). In some embodiments, SEQ ID NO: 72 is the amino acid sequence of 12A09_VHH16. In some embodiments, the first sdAb, the second sdAb or both consists of SEQ ID NO: 72. In some embodiments, the first sdAb, the second sdAb or both comprises or consists of a sequence with at least 70, 75, 80, 85, 90, 93, 95, 97, or 99% homology to SEQ ID NO: 72. Each possibility represents a separate embodiment of the invention. In some embodiments, the sequence with homolog comprises the CDRs of SEQ ID NO: 72.

In some embodiments, the first sdAb, the second sdAb or both comprises the amino acid sequence EVQLVESGGGLVQPGGSLRLSCKASGSTASIKTMAWYRQAPGKGLELVTAIASDN RKYYADSVKGRFTISRDNSKTTVYLQMNSLRAEDTAVYYCVVDVTKEDYWYWG QGTLVTVSS (SEQ ID NO: 73). In some embodiments, SEQ ID NO: 73 is the amino acid sequence of 12A9_VHH17. In some embodiments, the first sdAb, the second sdAb or both consists of SEQ ID NO: 73. In some embodiments, the first sdAb, the second sdAb or both comprises or consists of a sequence with at least 70, 75, 80, 85, 90, 93, 95, 97, or 99% homology to SEQ ID NO: 73. Each possibility represents a separate embodiment of the invention. In some embodiments, the sequence with homolog comprises the CDRs of SEQ ID NO: 73.

In some embodiments, the first sdAb, the second sdAb or both comprises the amino acid sequence EVQLVESGGGLVQPGGSLRLSCAASGSIASIKTMAWYRQAPGKGRELVTAIASDN RKYYADSVKGRFTISRDNSKTTVYLQMNSLRAEDTAVYYCVVDVTKEDYWYWG QGTLVTVSS (SEQ ID NO: 74). In some embodiments, SEQ ID NO: 74 is the amino acid sequence of 12A09_VHH18. In some embodiments, the first sdAb, the second sdAb or both consists of SEQ ID NO: 74. In some embodiments, the first sdAb, the second sdAb or both comprises or consists of a sequence with at least 70, 75, 80, 85, 90, 93, 95, 97, or 99% homology to SEQ ID NO: 74. Each possibility represents a separate embodiment of the invention. In some embodiments, the sequence with homolog comprises the CDRs of SEQ ID NO: 74.

In some embodiments, the sdAb comprises the amino acid sequence EVQLVESGGGLVQAGESLRLSCAASGSIASIRTMAWYRQAPGSQRELVAAISSGRE VYYADSVKGRFTISRDNAKTTVYLQMNSLRPEDTAVYYCVVDMYWQDYWWWG QGTQVTVSS (SEQ ID NO: 33). In some embodiments, the sdAb consists of SEQ ID NO: 33. In some embodiments, SEQ ID NO: 33 is the amino acid sequence of VHH 9B3. In some embodiments, the sdAb comprises or consists of a sequence with at least 70, 75, 80, 85, 90, 93, 95, 97, or 99% homology to SEQ ID NO: 33. Each possibility represents a separate embodiment of the invention. In some embodiments, the sequence with homolog comprises the CDRs of SEQ ID NO: 33.

In some embodiments, the first sdAb, the second sdAb or both comprises the amino acid sequence EVQLVESGGGLVQPGESLRLSCAASGSIASIRTMAWYRQAPGSQRELVAAISSGRE VYYADSVKGRFTISRDNAKTTVYLQMNSLRAEDTAVYYCVVDMYWQDYWWW GQGTQVTVSS (SEQ ID NO: 75). In some embodiments, SEQ ID NO: 75 is the amino acid sequence of 9B03_VHH1. In some embodiments, the first sdAb, the second sdAb or both consists of SEQ ID NO: 75. In some embodiments, the first sdAb, the second sdAb or both comprises or consists of a sequence with at least 70, 75, 80, 85, 90, 93, 95, 97, or 99% homology to SEQ ID NO: 75. Each possibility represents a separate embodiment of the invention. In some embodiments, the sequence with homolog comprises the CDRs of SEQ ID NO: 75.

In some embodiments, the first sdAb, the second sdAb or both comprises the amino acid sequence EVQLVESGGGLVQPGGSLRLSCKASGSIASIRTMAWYRQAPGKGLELVAAISSGRE VYYADSVKGRFTISRDNSKTTVYLQMNSLRAEDTAVYYCVVDMYWQDYWWWG QGTLVTVSS (SEQ ID NO: 76). In some embodiments, SEQ ID NO: 76 is the amino acid sequence of 9B03_VHH12. In some embodiments, the first sdAb, the second sdAb or both consists of SEQ ID NO: 76. In some embodiments, the first sdAb, the second sdAb or both comprises or consists of a sequence with at least 70, 75, 80, 85, 90, 93, 95, 97, or 99% homology to SEQ ID NO: 76. Each possibility represents a separate embodiment of the invention. In some embodiments, the sequence with homolog comprises the CDRs of SEQ ID NO: 76.

In some embodiments, the first sdAb, the second sdAb or both comprises the amino acid sequence EVQLVESGGGLVQPGGSLRLSCKASGSTASIRTMAWYRQAPGKGLELVSAISSGRE VYYADSVKGRFTISRDNSKTTVYLQMNSLRAEDTAVYYCVVDMYWQDYWWWG QGTLVTVSS (SEQ ID NO: 77). In some embodiments, SEQ ID NO: 77 is the amino acid sequence of 9B03_VHH19. In some embodiments, the first sdAb, the second sdAb or both consists of SEQ ID NO: 77. In some embodiments, the first sdAb, the second sdAb or both comprises or consists of a sequence with at least 70, 75, 80, 85, 90, 93, 95, 97, or 99% homology to SEQ ID NO: 77. Each possibility represents a separate embodiment of the invention. In some embodiments, the sequence with homolog comprises the CDRs of SEQ ID NO: 77.

In some embodiments, the first sdAb, the second sdAb or both comprises the amino acid sequence EVQLVESGGGLVQPGGSLRLSCAASGSIASIRTMAWYRQAPGKGLELVSAISSGRE VYYADSVKGRFTISRDNSKTTVYLQMNSLRAEDTAVYYCVVDMYWQDYWWWG QGTLVTVSS (SEQ ID NO: 78). In some embodiments, SEQ ID NO: 78 is the amino acid sequence of 9B03_VHH20. In some embodiments, the first sdAb, the second sdAb or both consists of SEQ ID NO: 78. In some embodiments, the first sdAb, the second sdAb or both comprises or consists of a sequence with at least 70, 75, 80, 85, 90, 93, 95, 97, or 99% homology to SEQ ID NO: 78. Each possibility represents a separate embodiment of the invention. In some embodiments, the sequence with homolog comprises the CDRs of SEQ ID NO: 78.

In some embodiments, the sdAb comprises the amino acid sequence EVQLVESGGGLVQAGESLRLSCAASGSIASINSMGWYRQAPGSQRELVAAISDRSE KYYADSVKGRFTISRDNAKTTVYLQMNSLRPEDTAVYYCVVDHHHSDWWTWGQ GTQVTVSS (SEQ ID NO: 34). In some embodiments, the sdAb consists of SEQ ID NO: 34. In some embodiments, SEQ ID NO: 34 is the amino acid sequence of VHH 9A7. In some embodiments, the sdAb comprises or consists of a sequence with at least 70, 75, 80, 85, 90, 93, 95, 97, or 99% homology to SEQ ID NO: 34. Each possibility represents a separate embodiment of the invention. In some embodiments, the sequence with homolog comprises the CDRs of SEQ ID NO: 34.

It will be understood by a skilled artisan that for the purposes of immune stimulation, such as in the context of treating cancer for example, the sdAb will be non-antagonistic, or can be antagonistic so long as once introduced into an agent of the invention the antagonistic effect is lost or reduced to acceptable low levels. For the purposes of immune inhibition, such as in the context of autoimmune disease for example, the sdAb may also be antagonistic.

### Agents

By another aspect, there is provided an agent comprising at least two CD28 binding single domain antibodies (sdAbs).

In some embodiments, the agent comprises a first sdAb and a second sdAb. In some embodiments, the at least two sdAbs are sdAbs of the invention. In some embodiments, the first sdAb is an sdAb of the invention. In some embodiments, the second sdAb is an sdAb of the invention.

In some embodiments, two sdAbs of the invention are linked by a linker. In some embodiments, a first sdAb and a second sdAb of the invention are linked by a linker. In some embodiments, the first sdAb is linked to the second sdAb by a linker. In some embodiments, this linkage produced an agent of the invention.

In some embodiments, the agent is a dimeric agent. As used herein, the term “dimeric” refers to an agent that includes two simpler molecules, e.g., monomers. In some embodiments, a dimeric agent includes two sdAbs. In some embodiments, the agent is monoparatopic. As used herein the term “monoparatopic” refers to an agent that targets only one epitope. In some embodiments, the agent comprises two sdAbs. In some embodiments, the dimer is a homodimer. In some embodiments, the dimer is a heterodimer. In some embodiments, the agent comprises a first sdAb and a second sdAb. In some embodiments, the agent comprises two identical sdAbs. In some embodiments, the two sdAbs comprise the same sequence. In some embodiments, the sequence is an amino acid sequence. In some embodiments, the agent comprises two different sdAbs. In some embodiments, the two sdAbs comprise different sequences. In some embodiments, the two sdAbs comprise the same CDRs. In some embodiments, the two sdAbs comprise different CDRs. In some embodiments, the target epitope of the sdAbs is the CD28 stalk region. In some embodiments, the target epitope of the sdAbs is the CD28 cleavage site. In some embodiments, the target epitope is the site of CD28 protease mediated shedding.

In some embodiments, the agent inhibits proteolytic cleavage of CD28. In some embodiments, the agent is superior at inhibiting proteolytic cleavage as compared to either sdAb as a monomer. In some embodiments, superior comprises increased inhibition. In some embodiments, increased is at least 5, 10, 15, 20, 25, 30, 35, 40, 45, 50, 55, 60, 65, 70, 75, 80, 85, 90, 95, 100, 150, 200, 250, 300, 400, or 500% increased. Each possibility represents a separate embodiment of the invention.

In some embodiments, the agent is not an antagonist of CD28. In some embodiments, each sdAb when not part of the agent is not an antagonist of CD28. In some embodiments, the first sdAb, the second sdAb or both when not part of the agent is an antagonist, and the agent is not an antagonist of CD28. In some embodiments, each sdAb when not part of the agent is an antagonist, and the agent is not an antagonist of CD28. In some embodiments, the agent, and/or the sdAb binds to CD28 but does not activate or block activation of the receptor. In some embodiments, the agent, and/or the sdAb does not block binding of a ligand to CD28. In some embodiments, the agent, does not inhibit binding of a ligand to CD28. In some embodiments, the agent lowly inhibits binding of a ligand to CD28.

In some embodiments, the agent is not an agonist of CD28. In some embodiments, each sdAb when not part of the agent is not an agonist of CD28. In some embodiments, the agent does not bind the ligand binding domain of mCD28. In some embodiments, the agent does not obscure or block access to the ligand binding domain. In some embodiments, the agent does not bind, obscure or block access to the IgV domain of sCD28.

In some embodiments, the agent comprises a first sdAb and a second sdAb. In some embodiments, the agent comprises two identical sdAbs. In some embodiments, the two sdAbs comprise the same sequence. In some embodiments, the sequence is an amino acid sequence. In some embodiments, the agent comprises two different sdAbs. In some embodiments, the two sdAbs comprise different sequences. In some embodiments, the two sdAbs comprise the same CDRs. In some embodiments, the two sdAbs comprise different CDRs. In some embodiments, the target epitope of the sdAbs is the CD28 stalk region. In some embodiments, the target epitope of the sdAbs is the CD28 cleavage site. In some embodiments, the target epitope is the site of CD28 protease mediated shedding.

In some embodiments, the agent comprises at least two sdAbs. In some embodiments, the agent comprises a plurality of sdAbs. In some embodiments, the agent comprises at least 2, 3, 4, 5, 6, or 7 sdAbs, Each possibility represents a separate embodiment of the invention. In some embodiments, the agent comprises two sdAbs. In some embodiments, the agent comprises a first sdAb and a second sdAb. In some embodiments, the first sdAb and the second sdAb are the same sdAb. In some embodiments, the first sdAb and the second sdAb comprise the same sequence. In some embodiments, the first sdAb and the second sdAb are different sdAbs. In some embodiments, the first sdAb and the second sdAb comprise different sequences.

In some embodiments, the first sdAb and the second sdAb bind the same mCD28 molecule. In some embodiments, the first sdAb and the second sdAb bind a single mCD28 molecule. In some embodiments, the first sdAb and the second sdAb bind different CD28 molecules. In some embodiments, the first sdAb binds a first CD28 molecule and the second sdAb binds a second CD28 molecule. In some embodiments, the first CD28 molecule and the second CD28 molecule are the same molecule. In some embodiments, the first CD28 molecule and the second CD28 molecule are different molecules.

In some embodiments, the first sdAb, the second sdAb or both comprises the amino acid sequence EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSS (SEQ ID NO: 40). In some embodiments, the first sdAb, the second sdAb or both consists of SEQ ID NO: 40. In some embodiments, SEQ ID NO: 40 is the amino acid sequence of VHH 2A1. In some embodiments, the first sdAb, the second sdAb or both comprises or consists of a sequence with at least 70, 75, 80, 85, 90, 93, 95, 97, or 99% homology to SEQ ID NO: 40. Each possibility represents a separate embodiment of the invention. In some embodiments, the sequence with homolog comprises the CDRs of SEQ ID NO: 40.

In some embodiments, the first sdAb, the second sdAb or both comprises the amino acid sequence EVQLVESGGGLVQAGGSLRLSCAASGSLFSINAMAWYRQAPGKQRELVAAITSSG STNYANSVKGRFTVSRDNAKNTMYLQMNSLKPEDTAVYYCVVDEYGSDYWIWG QGTQVTVSS (SEQ ID NO: 95). In some embodiments, the first sdAb, the second sdAb or both consists of SEQ ID NO: 95. In some embodiments, the first sdAb, the second sdAb or both comprises or consists of a sequence with at least 70, 75, 80, 85, 90, 93, 95, 97, or 99% homology to SEQ ID NO: 95. Each possibility represents a separate embodiment of the invention. In some embodiments, the sequence with homolog comprises the CDRs of SEQ ID NO: 95.

In some embodiments, the first sdAb, the second sdAb or both comprises the amino acid sequence QVQLVESGGGLVQAGGSLRLSCAASGSIFSINAMGWYRQAPGKQRERVAAITSGG STNYADSVKGRFTISRDNAKNTVYLQMNNLEPRDAGVYYCVVDLYGEDYWIWG QGTQVTVSS (SEQ ID NO: 96). In some embodiments, the first sdAb, the second sdAb or both consists of SEQ ID NO: 96. In some embodiments, the first sdAb, the second sdAb or both comprises or consists of a sequence with at least 70, 75, 80, 85, 90, 93, 95, 97, or 99% homology to SEQ ID NO: 96. Each possibility represents a separate embodiment of the invention. In some embodiments, the sequence with homolog comprises the CDRs of SEQ ID NO: 96.

In some embodiments, the first sdAb, the second sdAb or both comprises three CDRs: wherein CDR1 comprises the amino acid sequence INAMG (SEQ ID NO: 4), CDR2 comprises the amino acid sequence AISGGGDTYYADSVKG (SEQ ID NO: 5), and CDR3 comprises the amino acid sequence DLYGSDYWD (SEQ ID NO: 3). In some embodiments, the CDRs of SEQ ID NO: 1 are SEQ ID Nos: 3-5. In some embodiments, the first sdAb, the second sdAb or both comprises three CDRs: wherein CDR1 comprises the amino acid sequence INAMA (SEQ ID NO: 98), CDR2 comprises the amino acid sequence AITSSGSTNYANSVKG (SEQ ID NO: 99), and CDR3 comprises the amino acid sequence DEYGSDYWI (SEQ ID NO: 100). In some embodiments, the CDRs of SEQ ID NO: 95 are SEQ ID Nos: 98-100. In some embodiments, the first sdAb, the second sdAb or both comprises three CDRs: wherein CDR1 comprises the amino acid sequence INAMG (SEQ ID NO: 4), CDR2 comprises the amino acid sequence AITSGGSTNYADSVKG (SEQ ID NO: 101), and CDR3 comprises the amino acid sequence DLYGEDYWI (SEQ ID NO: 102). In some embodiments, the CDRs of SEQ ID NO: 96 are SEQ ID Nos: 4, 101 and 102.

It will be understood by a skilled artisan that any sdAb that binds to mCD28 on cells and inhibits proteolytic cleavage and sCD28 shedding can be employed as an sdAb of the invention. For the purposes of immune stimulation, such as in the context of cancer for example, the sdAb will also be non-antagonistic, or can be antagonistic so long as once introduced into an agent of the invention the antagonistic effect is lost or reduced to acceptable low levels. For the purposes of immune inhibition, such as in the context of autoimmune disease for example, the sdAb may also be antagonistic, or may become antagonistic once part of an agent of the invention.

In some embodiments, the two CD28 binding sdAbs are linked by a linker. In some embodiments, the first sdAb and the second sdAb are linked by a linker. In some embodiments, the first sdAb is linked to the second sdAb by a linker.

### Polypeptide Dimers

In some embodiments, the agent comprises a first polypeptide comprising the first sdAb. In some embodiments, the agent comprises a second polypeptide comprising the second sdAb. In some embodiments, the linker links the first polypeptide and the second polypeptide. In some embodiments, a polypeptide is a polypeptide chain. In some embodiments, the agent comprises the first polypeptide and the second polypeptide linked by a linker. In some embodiments, agents with two polypeptides are not CD28 antagonists.

In some embodiments, the first polypeptide comprises a signal peptide. In some embodiments, the second polypeptide comprises a signal peptide. In some embodiments, the first polypeptide is devoid of a signal peptide. In some embodiments, the second polypeptide is devoid of a signal peptide. It will be understood by a skilled artisan that the active form of the agent will not comprise signal peptides, but in order to express the polypeptides in cells it may be necessary to produce the polypeptides with signal peptides to facilitate secretion of the polypeptides from the cells. In some embodiments, the signal peptide is an Ig signal peptide. Any signal peptide that allows for production of the polypeptides of the invention may be employed.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the linkage is a C-terminal to C-terminal linkage. In some embodiments, C-terminal is a linkage at the most C-terminal amino acid of a polypeptide. In some embodiments, C-terminal is a linkage at the most C-terminal domain of a polypeptide. In some embodiments, the linkage is from a C-terminal domain to a C-terminal domain.

In some embodiments, the first polypeptide comprises a first cysteine amino acid. In some embodiments, the second polypeptide comprises a second cysteine amino acid. In some embodiments, the cysteine is a free cysteine. In some embodiments, the cysteine is outside of the sdAb. In some embodiments, the polypeptide comprises a cysteine outside of the sdAb. In some embodiments, the cysteine is a C-terminal cysteine. In some embodiments, the cysteine is an N-terminal cysteine. In some embodiments, the cysteine is a C-terminal amino acid. In some embodiments, the cysteine is in a C-terminal domain. In some embodiments, the cysteine is in a domain C-terminal to the sdAb. In some embodiments, the sdAb is N-terminal to the cysteine. In some embodiments, the linking comprises a bond between the first cysteine and the second cysteine. In some embodiments, the linker is a bond. In some embodiments, the bond is a disulfide bond. In some embodiments, the bond is between the most C-terminal cysteines in each polypeptide.

In some embodiments, the first polypeptide comprises EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSSC (SEQ ID NO: 113). In some embodiments, the first polypeptide consists of SEQ ID NO: 113. In some embodiments, the second polypeptide comprises SEQ ID NO: 113. In some embodiments, the second polypeptide consists of SEQ ID NO: 113. In some embodiments, the agent comprises a dimer of SEQ ID NO: 113. In some embodiments, the agent consists of a dimer of SEQ ID NO: 113. In some embodiments, the first polypeptide, the second polypeptide or both comprises or consists of a sequence with at least 70, 75, 80, 85, 90, 93, 95, 97, or 99% homology to SEQ ID NO: 113. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 113. Each possibility represents a separate embodiment of the invention. In some embodiments, the sequence with homolog comprises the CDRs of SEQ ID NO: 40.

In some embodiments, an agent comprising SEQ ID NO: 113 or a homolog thereof is not a CD28 antagonist. In some embodiments, an agent consisting of a dimer of SEQ ID NO: 113 or a homolog thereof is not a CD28 antagonist. In some embodiments, a CD28 antagonist is a substantial antagonist.

In some embodiments, an agent comprising linked cysteines is not a CD28 antagonist. In some embodiments, an agent comprising two polypeptides each with a C-terminal free cysteine wherein the free cysteines are linked is not a CD28 antagonist. In some embodiments, a CD28 antagonist is a substantial antagonist.

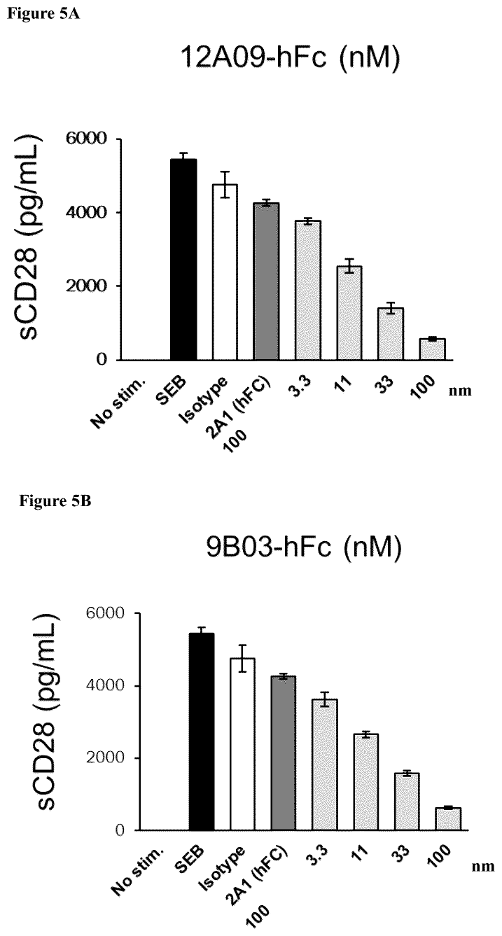

In some embodiments, the linker is a chemical linker. In some embodiments, the linker is an artificial linker. In some embodiments, the linker is not an amino acid linker. In some embodiments, the linker is not just a bond. In some embodiments, the linker comprises a biocompatible polymer. In some embodiments, the biocompatible polymer is at least partially biodegradable. In some embodiments, the biocompatible polymer is or comprises a polyglycol ether, a polyester, a polyamide or any combination thereof. In some embodiments, the polyglycol ether is or comprises polyethylene glycol (PEG). In some embodiments, the linker of the invention comprises PEG. In some embodiments, the linker of the invention comprises PEG characterized by Mn of between 100 and 5000 Da including any range between. In some embodiments, the PEG linker comprises at least 10 repeats of PEG. In some embodiments, the PEG linker comprises at least 1 repeat of PEG. In some embodiments, the PEG linker comprises at least 1, 2, 3, 4, 5, 6, 7, 8, 9, 10 or 11 repeats of PEG. Each possibility represents a separate embodiment of the invention. In some embodiments, the PEG linker comprises at least 11 repeats of PEG. In some embodiments, the PEG linker comprises 11 repeats of PEG. In some embodiments, the PEG linker is the linker provided in FIG. 5. In some embodiments, the PEG linker comprises or consists of Maleimide-N—(CH2)2-CO—NH—(CH2)2-(O—CH2CH2)n-(CH2)2-NH—CO—(CH2)2-N-Maleimide. In some embodiments, the PEG linker comprises or consists of N—(CH2)2-CO—NH—(CH2)2-(O—CH2CH2)n-(CH2)2-NH—CO—(CH2)2-N.

In some embodiments, an agent comprising cysteines linked by a chemical linker is not a CD28 antagonist. In some embodiments, an agent consisting of two polypeptides each comprising a cysteine linked by a chemical linker is not a CD28 antagonist. In some embodiments, a CD28 antagonist is a substantial antagonist. In some embodiments, the chemical linker is linked to a cysteine. In some embodiments, the chemical linker links the first and second cysteine. In some embodiments, the chemical linker is linked to a cysteine by a maleimide reactive group. In some embodiments, the reaction is to a thiol in the polypeptide. In some embodiments, the reaction is to a thiol in the cysteine. Methods of generating chemical linkers and attaching them to polypeptides are well known in the art and any such method may be employed to produce the agent of the invention.

In some embodiments, the first polypeptide further comprises a first dimerization domain. In some embodiments, the second polypeptide further comprises a second dimerization domain. In some embodiments, the dimerization domain is C-terminal to the sdAb. In some embodiments, the dimerization domain is N-terminal to the sdAb. In some embodiments, the sdAb is N-terminal to the dimerization domain. In some embodiments, the dimerization domain is a C-terminal domain. In some embodiments, the dimerization domain comprises the cysteine. In some embodiments, the linker comprises the dimerization domains. In some embodiments, the linker comprises the bonds between the dimerization domains. In some embodiments, the bonds are disulfide bonds.

In some embodiments, the dimerization domains are capable of dimerizing with each other. In some embodiments, the first dimerization domain is capable of dimerization with the second dimerization domain. In some embodiments, the first and second dimerization domains are capable of dimerizing with each other. In some embodiments, capable of dimerizing is configured to dimerize. In some embodiments, dimerization is under physiological conditions. In some embodiments, dimerization is within a bodily fluid. In some embodiments, the bodily fluid is blood. In some embodiments, the bodily fluid is plasma. In some embodiments, the bodily fluid is serum. In some embodiments, dimerization is within a subject. In some embodiments, dimerization is in vivo. In some embodiments, dimerization is in vitro.

As used herein, the term “dimerization domain” refers to an amino acid sequence that upon contacting another amino acid sequence (the other dimerization domain) binds to it to form a dimer. Dimerization domains are well known in the art, as many protein sequences are known to bind to each other. In some embodiments, dimerization comprises formation of a covalent bond between the dimerization domains. In some embodiments, dimerization comprises electrostatic binding. In some embodiments, dimerization does not comprise electrostatic binding. In some embodiments, dimerization is reversible. In some embodiments, dimerization is irreversible. In some embodiments, dimerization comprises a bond forming between the dimerization domains. In some embodiments, the bond is a chemical bond. In some embodiments, the bond is a disulfide bond. In some embodiments, the bond is a peptide bond. Examples of dimerization domain include the hinge domain of antibody heavy chains, the CH1/CL domains of antibody heavy/light chains, and the ECD domains of TCR alpha/beta to name but a few.

In some embodiments, the dimerization domain comprises or consists of an immunoglobulin (Ig) hinge domain. In some embodiments, an agent comprising an Ig hinge domain is not a CD28 antagonist. In some embodiments, a CD28 antagonist is a substantial antagonist. In some embodiments, the first dimerization domain is a first Ig hinge domain. In some embodiments, the second dimerization domain is a second Ig hinge domain. In some embodiments, an Ig hinge domain is a heavy chain hinge domain. In some embodiments, the Ig is a human Ig. In some embodiments, the immunoglobulin is elected from IgA, IgD, IgE, IgG and IgM. In some embodiments, the immunoglobulin is IgG. In some embodiments, the IgG is IgG1. In some embodiments, the IgG is IgG2. In some embodiments, the IgG is IgG3. In some embodiments, the IgG is selected from IgG1 and IgG3. In some embodiments, the IgG is IgG4. In some embodiments, the IgG is human IgG. In some embodiments, the first and second dimerization domains are both Ig hinge domains. In some embodiments, the first and second dimerization domains are identical. In some embodiments, the first and second dimerization domains are at least 95% identical. In some embodiments, the first and second dimerization domains are at least 50, 55, 60, 65, 70, 75, 80, 85, 90, 95, 97, 99 or 100% identical. Each possibility represents a separate embodiment of the invention.

In some embodiments, the Ig hinge domain comprises the amino acid sequence DKTHTCPPCPAPEL (SEQ ID NO: 38). In some embodiments, the Ig hinge domain consists of SEQ ID NO: 38. In some embodiments, the IgG1 hinge domain comprises or consists of SEQ ID NO: 38. In some embodiments, the Ig hinge domain comprises the amino acid sequence DKTHTCPPCPAPE (SEQ ID NO: 83). In some embodiments, the Ig hinge domain consists of SEQ ID NO: 83. In some embodiments, the IgG1 hinge domain comprises or consists of SEQ ID NO: 83. In some embodiments, the hinge domain comprises the amino acid sequence EPKSCDKTHTCPPCPAPELLGG (SEQ ID NO: 50). In some embodiments, the hinge domain consists of the amino acid sequence of SEQ ID NO: 50. In some embodiments, the IgG1 hinge comprises or consists of SEQ ID NO: 50. In some embodiments, the hinge domain comprises the amino acid sequence EPKCCVECPPCPAPPAAA (SEQ ID NO: 51). In some embodiments, the hinge domain consists of the amino acid sequence of SEQ ID NO: 51. In some embodiments, the IgG2 hinge comprises or consists of SEQ ID NO: 51. In some embodiments, the hinge domain comprises the amino acid sequence EPKCCVECPPCPAPPVAGP (SEQ ID NO: 84). In some embodiments, the hinge domain consists of the amino acid sequence of SEQ ID NO: 84. In some embodiments, the IgG2 hinge comprises or consists of SEQ ID NO: 84. In some embodiments, the hinge domain comprises the amino acid sequence ESKYGPPCPPCPAPEFLGG (SEQ ID NO: 52). In some embodiments, the hinge domain consists of the amino acid sequence of SEQ ID NO: 52. In some embodiments, the IgG4 hinge comprises or consists of SEQ ID NO: 52. In some embodiments, the hinge domain comprises the amino acid sequence ESKYGPPCPPCPAPEFEGG (SEQ ID NO: 85). In some embodiments, the hinge domain consists of the amino acid sequence of SEQ ID NO: 85. In some embodiments, the IgG4 hinge comprises or consists of SEQ ID NO: 85. In some embodiments, the hinge domain comprises the amino acid sequence ESKYGPPCPSCPAPEFLGG (SEQ ID NO: 86). SEQ ID NO: 85 includes the S228P and L235E mutations which are well known in the art. SEQ ID NO: 85 is SEQ ID NO: 86 comprising the mutations. These mutations are known to reduce aggregation, enhance stability and reduce effector function. In some embodiments, the hinge domain consists of the amino acid sequence of SEQ ID NO: 86. In some embodiments, the IgG4 hinge comprises or consists of SEQ ID NO: 86. In some embodiments, the hinge domain comprises the amino acid sequence ELKTPLGDTTHTCPRCPEPKSCDTPPPCPRCPEPKSCDTPPPCPRCPEPKSCDTPPPC PRCPAPELLGGP (SEQ ID NO: 53). In some embodiments, the hinge domain consists of the amino acid sequence of SEQ ID NO: 53. In some embodiments, the IgG3 hinge comprises or consists of SEQ ID NO: 53. In some embodiments, the hinge domain comprises a CPXCP (SEQ ID NO: 54) motif. In some embodiments, SEQ ID NO: 54 is the hinge domain core. In some embodiments, the X in SEQ ID NO: 54 is selected from P and R. In some embodiments, SEQ ID NO: 54 is CPPCP (SEQ ID NO: 55). In some embodiments, the IgG1 core consists of SEQ ID NO: 55. In some embodiments, the IgG2 core comprises SEQ ID NO: 55. In some embodiments, the IgG2 core consists of CCVECPPCP (SEQ ID NO: 87). In some embodiments, the IgG4 core comprises or consists of SEQ ID NO: 55. In some embodiments, SEQ ID NO: 54 is CPRCP (SEQ ID NO: 56). In some embodiments, the IgG3 core comprises or consists of SEQ ID NO: 56. In some embodiments, SEQ ID NO: 54 is CPSCP (SEQ ID NO: 88). In some embodiments, the IgG4 core comprises or consists of SEQ ID NO: 88. It will thus be understood that the cysteines of the hinge domain found in SEQ ID NO: 54 are required for disulfide bonding and dimerization.

In some embodiments, the first polypeptide comprises EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSSDKTHTCPPCPAPEL (SEQ ID NO: 114). In some embodiments, the first polypeptide consists of SEQ ID NO: 114. In some embodiments, the second polypeptide comprises SEQ ID NO: 114. In some embodiments, the second polypeptide consists of SEQ ID NO: 114. In some embodiments, the agent comprises a dimer of SEQ ID NO: 114. In some embodiments, the agent consists of a dimer of SEQ ID NO: 114. In some embodiments, the first polypeptide, the second polypeptide or both comprises or consists of a sequence with at least 70, 75, 80, 85, 90, 93, 95, 97, or 99% homology to SEQ ID NO: 114. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 114. Each possibility represents a separate embodiment of the invention. In some embodiments, the sequence with homolog comprises the CDRs of SEQ ID NO: 40.

In some embodiments, an agent comprising SEQ ID NO: 114 or a homolog thereof is not a CD28 antagonist. In some embodiments, an agent consisting of a dimer of SEQ ID NO: 114 or a homolog thereof is not a CD28 antagonist. In some embodiments, a CD28 antagonist is a substantial antagonist.

In some embodiments, the dimerization domain further comprises a CH2 domain of an Ig. In some embodiments, the CH2 domain is of an Ig heavy chain. In some embodiments, the dimerization domain further comprises a CH3 domain of an Ig. In some embodiments, the CH3 domain is of an Ig heavy chain. In some embodiments, the first dimerization domain comprises a CH2 domain, a CH3 domain or both. In some embodiments, the second dimerization domain comprises a CH2 domain, a CH3 domain or both. In some embodiments, the Ig is IgG. In some embodiments, the IgG is IgG1. In some embodiments, the Ig is IgG2. In some embodiments, the IgG is IgG4. In some embodiments, the IgG is IgG3. In some embodiments, the IgG1 is modified IgG1. In some embodiments, the IgG3 is modified IgG3. In some embodiments, modified is modified to reduce effector function. In some embodiments, modified is modified to abolish effector function. In some embodiments, modified is PG-LALA modified.

In some embodiments, the dimerization domain comprises an Fc domain. In some embodiments, the dimerization domain consists of an Fc domain. In some embodiments, an Fc domain comprises the hinge, CH2 and CH3 domains. In some embodiments, an Fc domain consists of the hinge, CH2 and CH3 domains. In some embodiments, an agent comprising an Fc domain is not a CD28 antagonist.

In some embodiments, a CH2 domain comprises the amino acid sequence LGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAK (SEQ ID NO: 57). In some embodiments, the CH2 domain consists of SEQ ID NO: 57. In some embodiments, SEQ ID NO: 57 is the IgG1 CH2 domain. In some embodiments, a CH2 domain comprises the amino acid sequence PSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAK (SEQ ID NO: 58). In some embodiments, the CH2 domain consists of SEQ ID NO: 58. In some embodiments, SEQ ID NO: 58 is the IgG1 CH2 domain. In some embodiments, the CH2 domain comprises or consists of a sequence with at least 70, 75, 80, 85, 90, 93, 95, 97, or 99% homology to SEQ ID NO: 57. Each possibility represents a separate embodiment of the invention. In some embodiments, the CH2 domain comprises or consists of a dimer of a polypeptide a sequence with at least 70, 75, 80, 85, 90, 93, 95, 97, or 99% homology to SEQ ID NO: 58. Each possibility represents a separate embodiment of the invention. In some embodiments, a CH2 domain comprises the amino acid sequence PSVFLFPPKPKDTLMISRTPEVTCVVVDVSQEDPEVQFNWYVDGVEVHNAKTKPR EEQFNSTYRVVSVLTVLHQDWLNGKEYKCKVSNKGLPSSIEKTISKAK (SEQ ID NO:89). In some embodiments, the CH2 domain consists of SEQ ID NO: 89. In some embodiments, SEQ ID NO: 89 is the IgG4 CH2 domain. In some embodiments, the CH2 domain consists of SEQ ID NO: 89. In some embodiments, SEQ ID NO: 89 is the IgG4 CH2 domain. In some embodiments, the CH2 domain comprises or consists of a sequence with at least 70, 75, 80, 85, 90, 93, 95, 97, or 99% homology to SEQ ID NO: 89. Each possibility represents a separate embodiment of the invention. In some embodiments, the CH2 domain comprises or consists of a dimer of a polypeptide a sequence with at least 70, 75, 80, 85, 90, 93, 95, 97, or 99% homology to SEQ ID NO: 89. Each possibility represents a separate embodiment of the invention.

In some embodiments, a CH3 domain comprises the amino acid sequence GQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 59). In some embodiments, the CH3 domain consists of SEQ ID NO: 59. In some embodiments, SEQ ID NO: 59 is the IgG1 CH3 domain. In some embodiments, CH3 domain comprises or consists of a sequence with at least 70, 75, 80, 85, 90, 93, 95, 97, or 99% homology to SEQ ID NO: 59. Each possibility represents a separate embodiment of the invention. In some embodiments, a CH3 domain comprises the amino acid sequence GQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGK (SEQ ID NO: 90). In some embodiments, the CH3 domain consists of SEQ ID NO: 90. In some embodiments, SEQ ID NO: 90 is the IgG4 CH3 domain. In some embodiments, CH3 domain comprises or consists of a sequence with at least 70, 75, 80, 85, 90, 93, 95, 97, or 99% homology to SEQ ID NO: 90. Each possibility represents a separate embodiment of the invention.

In some embodiments, the Fc domain comprises DKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALP APIEKTISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYTQKSL SLSPGK (SEQ ID NO: 60). In some embodiments, the Fc domain consists of SEQ ID NO: 60. In some embodiments, the Fc domain of IgG1 comprises or consists of SEQ ID NO: 60. In some embodiments, the Fc domain comprises or consists of a sequence with at least 70, 75, 80, 85, 90, 93, 95, 97, or 99% homology to SEQ ID NO: 60. Each possibility represents a separate embodiment of the invention. In some embodiments, the Fc domain comprises ESKYGPPCPSCPAPEFLGGPSVFLFPPKPKDTLMISRTPEVTCVVVDVSQEDPEVQF NWYVDGVEVHNAKTKPREEQFNSTYRVVSVLTVLHQDWLNGKEYKCKVSNKGL PSSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 91). In some embodiments, the Fc domain consists of SEQ ID NO: 91. In some embodiments, the Fc domain of IgG4 comprises or consists of SEQ ID NO: 91. In some embodiments, the Fc domain comprises or consists of a sequence with at least 70, 75, 80, 85, 90, 93, 95, 97, or 99% homology to SEQ ID NO: 91. Each possibility represents a separate embodiment of the invention. In some embodiments, SEQ ID NO: 91 comprises mutation of S10 to P. In some embodiments, SEQ ID NO: 91 comprises mutation of L17 to E.

In some embodiments, the dimerization domain does not induce antibody-dependent cell-mediated cytotoxicity (ADCC). In some embodiments, the dimerization domain does not induce complement-dependent cytotoxicity (CDC). In some embodiments, the dimerization domain is configured not to induce ADCC or CDC. In some embodiments, the dimerization domain is configured to have reduced ADCC or CDC. In some embodiments, the dimerization domain does not possess effector function. In some embodiments, the dimerization domain comprises reduced effector function. In some embodiments, the dimerization domain comprises at least one mutation that reduces or abolishes effector function. In some embodiments, the dimerization domain comprises at least one mutation that reduces ADCC or CDC. In some embodiments, the dimerization domain comprises at least one mutation that reduces effector function. In some embodiments, reduced CDC, ADCC or effector function comprises at least one mutation that reduces CDC, ADC or effector function.

It will be known by a skilled artisan that IgG2 and IgG4 possess greatly reduced effector function and are not generally cytotoxic in nature. Additionally, mutations such as S228P and L235E in IgG4 are known to reduce effector function even more. Further, mutations that reduce the cytotoxicity/effector function of IgG1 and IgG3 are well known in the art. In some embodiments, the IgG comprises at least one mutation. In some embodiments, the mutation is a plurality of mutations. In some embodiments, the mutation decreases cytotoxicity. In some embodiments, the mutation increases stability. In some embodiments, the mutation decreases aggregation. In some embodiments, the plurality of mutations that decreases cytotoxicity comprise the PG-LALA mutations. In some embodiments, the mutation is mutation of proline 329 of the IgG1 human heavy chain to glycine (P329G). In some embodiments, the P to G mutation is mutation of P109 of SEQ ID NO: 60 to G. In some embodiments, the mutation is mutation of leucine 234 of the IgG1 human heavy chain to alanine (L234A). In some embodiments, the L to A mutation is mutation of L14 of SEQ ID NO: 60 to A. In some embodiments, the mutation is mutation of leucine 235 of the IgG1 human heavy chain to alanine (L235A). In some embodiments, the L to A mutation is mutation of L15 of SEQ ID NO: 60 to A. In some embodiments, the plurality of mutation comprises P109G, L14A and L15A of SEQ ID NO: 60. In some embodiments, the plurality of mutation comprises P329G, L234A and L235A of the IgG1 human heavy chain. It will be understood by a skilled artisan that parallel mutation can also be performed in the IgG3 heavy chain or the heavy chains of non-human IgG1s. In some embodiments, the mutation is mutation of leucine 235 of the IgG4 human heavy chain to glutamic acid (L235E). n some embodiments, the mutation is mutation of serine 228 of the IgG4 human heavy chain to proline (S228P). It will be understood that the number given herein is in reference to a full-length IgG including the variable domains. The numbers can be shifted to correspond to the positions of these amino acids within just the Fc portion of the IgG.

In some embodiments, the dimerization domain comprising reduced cytotoxicity and/or effector function comprises DKTHTCPPCPAPEAAGGPSVFLFPPKPKDTLMISRTPEVTCVVVDVSHEDPEVKFN WYVDGVEVHNAKTKPREEQYNSTYRVVSVLTVLHQDWLNGKEYKCKVSNKAL GAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTQK SLSLSPGK (SEQ ID NO: 39). In some embodiments, the dimerization domain comprises SEQ ID NO: 39. In some embodiments, the dimerization domain consists of SEQ ID NO: 39. In some embodiments, the dimerization domain comprises or consists of a sequence with at least 70, 75, 80, 85, 90, 93, 95, 97, or 99% homology to SEQ ID NO: 39. Each possibility represents a separate embodiment of the invention. In some embodiments, the sequence with homolog comprises the P to G, L to A and L to A mutations. In some embodiments, the dimerization domain comprising reduced cytotoxicity and/or effector function comprises ESKYGPPCPPCPAPEFEGGPSVFLFPPKPKDTLMISRTPEVTCVVVDVSQEDPEVQF NWYVDGVEVHNAKTKPREEQFNSTYRVVSVLTVLHQDWLNGKEYKCKVSNKGL PSSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 92). In some embodiments, the Fc domain consists of SEQ ID NO: 92. In some embodiments, the Fc domain of IgG4 comprises or consists of SEQ ID NO: 92. In some embodiments, the Fc domain comprises or consists of a sequence with at least 70, 75, 80, 85, 90, 93, 95, 97, or 99% homology to SEQ ID NO: 92. Each possibility represents a separate embodiment of the invention. In some embodiments, the sequence with homology comprises the P at position 10. In some embodiments, the sequence with homolog comprises the E at position 17.

In some embodiments, the sdAb and the dimerization domain are separated by a linker. In some embodiments, the linker is an amino acid linker. In some embodiments, the linker is a peptide linker. In some embodiments, the linker is a peptide bond. In some embodiments, the first sdAb and the first dimerization domain are separated by a linker. In some embodiments, the second sdAb and second dimerization domain are separated by a linker. In some embodiments, both polypeptides comprise the linker. In some embodiments, either comprises the linker.

In some embodiments, the linker is an amino acid linker. In some embodiments, the linker is a flexible linker. In some embodiments, the linker is a hydrophilic linker. In some embodiments, the linker comprises the amino acid sequence GGGGS (SEQ ID NO: 118). In some embodiments, the linker comprises the amino acid sequence GS. In some embodiments, the linker comprises the amino acid sequence (GGGGS)n wherein n is an integer. In some embodiments, the linker comprises the amino acid sequence AAA(GGGGS)n wherein n is an integer. In some embodiments, the linker comprises the amino acid sequence (GS)n wherein n is an integer. In some embodiments, the linker comprises the amino acid sequence (GGS)n wherein n is an integer. In some embodiments, the linker comprises the amino acid sequence (GSGGS)n wherein n is an integer. In some embodiments, the linker comprises the amino acid sequence (EGGGS)n wherein n is an integer. In some embodiments, the linker comprises the amino acid sequence (EGGS)n wherein n is an integer. In some embodiments, n is an integer selected from 1, 2, 3, 4, 5, 6, 7, 8, 9 and 10. Each possibility represents a separate embodiment of the invention. In some embodiments, n is 1. In some embodiments, n is 2. In some embodiments, n is 3. In some embodiments, n is 4. In some embodiments, n is 5. In some embodiments, n is 7.

In some embodiments, n is 8. In some embodiments, the linker comprises at least 1, 2, 3, 4, 5, 6, 7, 8, 9, 10, 11, 12, 13, 14, 15, 16, 17, 18, 19, 20, 21, 22, 23, 24 or 25 amino acids. Each possibility represents a separate embodiment of the invention. In some embodiments, the linker comprises at least 1 amino acid. In some embodiments, the linker comprises at least 5 amino acids. In some embodiments, the linker comprises at least 8 amino acids. In some embodiments, the linker comprises at least 10 amino acids. In some embodiments, the linker comprises at least 13 amino acids. In some embodiments, the linker comprises at least 15 amino acids. In some embodiments, the linker comprises at least 18 amino acids. In some embodiments, the linker comprises at least 25 amino acids. In some embodiments, the linker comprises at least 35 amino acids. In some embodiments, the linker comprises at most 25, 28, 30, 35, 40, 45, 50, 60, 70, 75, 80, 90 or 100 amino acids. Each possibility represents a separate embodiment of the invention. In some embodiments, the linker comprises at most 25 amino acids. In some embodiments, the linker comprises at most 28 amino acids. In some embodiments, the linker comprises at most 35 amino acids. In some embodiments, the linker comprises at most 50 amino acids. In some embodiments, the linker comprises 1-50, 1-28, 1-25, 1-18, 1-15, 1-13, 1-10, 5-50, 5-28, 5-25, 5-18, 5-15, 5-13, 5-10, 10-50, 10-28, 10-25, 10-18, 10-15, 10-13, 15-50, 15-28, 15-25, 15-18, 25-50 and 28-50 amino acids. Each possibility represents a separate embodiment of the invention. In some embodiments, the linker comprises between 15 and 25 amino acids. In some embodiments, the linker comprises between 15 and 35 amino acids. In some embodiments, the linker comprises between 25 and 35 amino acids. In some embodiments, the linker comprises between 18 and 28 amino acids. In some embodiments, the linker comprises between 10 and 25 amino acids. In some embodiments, the linker comprises between 13 and 28 amino acids. In some embodiments, the linker comprises between 10 and 20 amino acids. In some embodiments, the linker comprises between 13 and 23 amino acids. It will be understood by a skilled artisan that in addition to increasing the number of repeats in the linker, the N- and C-termini can also include additional bases such as additional Gs and/or As.

In some embodiments, the first polypeptide comprises EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSSAAAGGGGSGGGGSGGGGSDKTHTCPPCPAPEAAGGPSVFLFPPKPK DTLMISRTPEVTCVVVDVSHEDPEVKFNWYVDGVEVHNAKTKPREEQYNSTYRV VSVLTVLHQDWLNGKEYKCKVSNKALGAPIEKTISKAKGQPREPQVYTLPPSRDE LTKNQVSLTCLVKGFYPSDIAVEWESNGQPENNYKTTPPVLDSDGSFFLYSKLTVD KSRWQQGNVFSCSVMHEALHNHYTQKSLSLSPGK (SEQ ID NO: 116). In some embodiments, the first polypeptide consists of SEQ ID NO: 116. In some embodiments, the second polypeptide comprises SEQ ID NO: 116. In some embodiments, the second polypeptide consists of SEQ ID NO: 116. In some embodiments, the agent comprises a dimer of SEQ ID NO: 116. In some embodiments, the agent consists of a dimer of SEQ ID NO: 116. In some embodiments, the first polypeptide, the second polypeptide or both comprises or consists of a sequence with at least 70, 75, 80, 85, 90, 93, 95, 97, or 99% homology to SEQ ID NO: 116. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 116. Each possibility represents a separate embodiment of the invention. In some embodiments, the sequence with homology comprises the CDRs of SEQ ID NO: 1. In some embodiments, the sequence with homolog comprises the P to G, L to A and L to A mutations of the Fc domain. In some embodiments, the sequence with homology comprises G244, A149 and A150 of SEQ ID NO: 116.

In some embodiments, an agent comprising SEQ ID NO: 116 or a homolog thereof is not a CD28 antagonist. In some embodiments, an agent consisting of a dimer of SEQ ID NO: 116 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSSAAAGGGGSGGGGSGGGGSGGGGSGGGGSDKTHTCPPCPAPEAAGG PSVFLFPPKPKDTLMISRTPEVTCVVVDVSHEDPEVKFNWYVDGVEVHNAKTKPR EEQYNSTYRVVSVLTVLHQDWLNGKEYKCKVSNKALGAPIEKTISKAKGQPREPQ VYTLPPSRDELTKNQVSLTCLVKGFYPSDIAVEWESNGQPENNYKTTPPVLDSDGS FFLYSKLTVDKSRWQQGNVFSCSVMHEALHNHYTQKSLSLSPGK (SEQ ID NO: 117). In some embodiments, the first polypeptide consists of SEQ ID NO: 117. In some embodiments, the second polypeptide comprises SEQ ID NO: 117. In some embodiments, the second polypeptide consists of SEQ ID NO: 117. In some embodiments, the agent comprises a dimer of SEQ ID NO: 117. In some embodiments, the agent consists of a dimer of SEQ ID NO: 117. In some embodiments, the first polypeptide, the second polypeptide or both comprises or consists of a sequence with at least 70, 75, 80, 85, 90, 93, 95, 97, or 99% homology to SEQ ID NO: 117. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 117. Each possibility represents a separate embodiment of the invention. In some embodiments, the sequence with homology comprises the CDRs of SEQ ID NO: 1. In some embodiments, the sequence with homolog comprises the P to G, L to A and L to A mutations of the Fc domain. In some embodiments, the sequence with homology comprises G254, A159 and A160 of SEQ ID NO: 117.

In some embodiments, an agent comprising SEQ ID NO: 117 or a homolog thereof is not a CD28 antagonist. In some embodiments, an agent consisting of a dimer of SEQ ID NO: 117 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSSDKTHTCPPCPAPEAAGGPSVFLFPPKPKDTLMISRTPEVTCVVVDVS HEDPEVKFNWYVDGVEVHNAKTKPREEQYNSTYRVVSVLTVLHQDWLNGKEYK CKVSNKALGAPIEKTISKAKGQPREPQVYTLPPSRDELTKNQVSLTCLVKGFYPSDI AVEWESNGQPENNYKTTPPVLDSDGSFFLYSKLTVDKSRWQQGNVFSCSVMHEA LHNHYTQKSLSLSPGK (SEQ ID NO: 115). In some embodiments, the first polypeptide consists of SEQ ID NO: 115. In some embodiments, the second polypeptide comprises SEQ ID NO: 115. In some embodiments, the second polypeptide consists of SEQ ID NO: 115. In some embodiments, the agent comprises a dimer of SEQ ID NO: 115. In some embodiments, the agent consists of a dimer of SEQ ID NO: 115. In some embodiments, the first polypeptide, the second polypeptide or both comprises or consists of a sequence with at least 70, 75, 80, 85, 90, 93, 95, 97, or 99% homology to SEQ ID NO: 115. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 115. Each possibility represents a separate embodiment of the invention. In some embodiments, the sequence with homology comprises the CDRs of SEQ ID NO: 1. In some embodiments, the sequence with homolog comprises the P to G, L to A and L to A mutations of the Fc domain. In some embodiments, the sequence with homology comprises G1156, A131 and A132 of SEQ ID NO: 115.

In some embodiments, an agent comprising SEQ ID NO: 115 or a homolog thereof is not a CD28 antagonist. In some embodiments, an agent consisting of a dimer of SEQ ID NO: 115 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASINSMGWYRQAPGSQRELVAAINEKLL IYYADSVKGRFTISRDNAKTTVYLQMNSLRPEDTAVYYCVVDLYGSDYWDWGQ GTQVTVSSAAAGGGGSGGGGSGGGGSDKTHTCPPCPAPEAAGGPSVFLFPPKPKD TLMISRTPEVTCVVVDVSHEDPEVKFNWYVDGVEVHNAKTKPREEQYNSTYRVV SVLTVLHQDWLNGKEYKCKVSNKALGAPIEKTISKAKGQPREPQVYTLPPSRDEL TKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 61). In some embodiments, the first polypeptide consists of SEQ ID NO: 61. In some embodiments, the second polypeptide comprises SEQ ID NO: 61. In some embodiments, the second polypeptide consists of SEQ ID NO: 61. In some embodiments, the agent comprises a dimer of SEQ ID NO: 61. In some embodiments, the agent consists of a dimer of SEQ ID NO: 61. In some embodiments, the first polypeptide, the second polypeptide or both comprises or consists of a sequence with at least 70, 75, 80, 85, 90, 93, 95, 97, or 99% homology to SEQ ID NO: 61. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 61. Each possibility represents a separate embodiment of the invention. In some embodiments, the sequence with homology comprises the CDRs of SEQ ID NO: 26. In some embodiments, the sequence with homolog comprises the P to G, L to A and L to A mutations of the Fc domain. In some embodiments, the sequence with homology comprises G244, A149 and A150 of SEQ ID NO: 61.

In some embodiments, an agent comprising SEQ ID NO: 61 or a homolog thereof is not a CD28 antagonist. In some embodiments, an agent consisting of a dimer of SEQ ID NO: 61 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASINAMGWYRQAPGSQRELVAAISGGG DTYYADSVKGRFTISRDNAKTTVYLQMNSLRPEDTAVYYCVVDMIEQQWWYWG QGTQVTVSSAAAGGGGSGGGGSGGGGSDKTHTCPPCPAPEAAGGPSVFLFPPKPK DTLMISRTPEVTCVVVDVSHEDPEVKFNWYVDGVEVHNAKTKPREEQYNSTYRV VSVLTVLHQDWLNGKEYKCKVSNKALGAPIEKTISKAKGQPREPQVYTLPPSRDE LTKNQVSLTCLVKGFYPSDIAVEWESNGQPENNYKTTPPVLDSDGSFFLYSKLTVD KSRWQQGNVFSCSVMHEALHNHYTQKSLSLSPGK (SEQ ID NO: 62). In some embodiments, the first polypeptide consists of SEQ ID NO: 62. In some embodiments, the second polypeptide comprises SEQ ID NO: 62. In some embodiments, the second polypeptide consists of SEQ ID NO: 62. In some embodiments, the agent comprises a dimer of SEQ ID NO: 62. In some embodiments, the agent consists of a dimer of SEQ ID NO: 62. In some embodiments, the first polypeptide, the second polypeptide or both comprises or consists of a sequence with at least 70, 75, 80, 85, 90, 93, 95, 97, or 99% homology to SEQ ID NO: 62. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 62. Each possibility represents a separate embodiment of the invention. In some embodiments, the sequence with homology comprises the CDRs of SEQ ID NO: 27. In some embodiments, the sequence with homolog comprises the P to G, L to A and L to A mutations of the Fc domain. In some embodiments, the sequence with homology comprises G244, A149 and A150 of SEQ ID NO: 62.

In some embodiments, an agent comprising SEQ ID NO: 62 or a homolog thereof is not a CD28 antagonist. In some embodiments, an agent consisting of a dimer of SEQ ID NO: 62 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASINAMGWYRQAPGSQRELVAAISGGG DTYYADSVKGRFTISRDNAKTTVYLQMNSLRPEDTAVYYCVVDTHRGVYWYWG QGTQVTVSSAAAGGGGSGGGGSGGGGSDKTHTCPPCPAPEAAGGPSVFLFPPKPK DTLMISRTPEVTCVVVDVSHEDPEVKFNWYVDGVEVHNAKTKPREEQYNSTYRV VSVLTVLHQDWLNGKEYKCKVSNKALGAPIEKTISKAKGQPREPQVYTLPPSRDE LTKNQVSLTCLVKGFYPSDIAVEWESNGQPENNYKTTPPVLDSDGSFFLYSKLTVD KSRWQQGNVFSCSVMHEALHNHYTQKSLSLSPGK (SEQ ID NO: 63). In some embodiments, the first polypeptide consists of SEQ ID NO: 63. In some embodiments, the second polypeptide comprises SEQ ID NO: 63. In some embodiments, the second polypeptide consists of SEQ ID NO: 63. In some embodiments, the agent comprises a dimer of SEQ ID NO: 63. In some embodiments, the agent consists of a dimer of SEQ ID NO: 63. In some embodiments, the first polypeptide, the second polypeptide or both comprises or consists of a sequence with at least 70, 75, 80, 85, 90, 93, 95, 97, or 99% homology to SEQ ID NO: 63. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 63. Each possibility represents a separate embodiment of the invention. In some embodiments, the sequence with homology comprises the CDRs of SEQ ID NO: 28. In some embodiments, the sequence with homolog comprises the P to G, L to A and L to A mutations of the Fc domain. In some embodiments, the sequence with homology comprises G244, A149 and A150 of SEQ ID NO: 63.

In some embodiments, an agent comprising SEQ ID NO: 63 or a homolog thereof is not a CD28 antagonist. In some embodiments, an agent consisting of a dimer of SEQ ID NO: 63 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASIKTMAWYRQAPGSQRELVAAINYIKE IYYADSVKGRFTISRDNAKTTVYLQMNSLRPEDTAVYYCVVDVTKEDYWYWGQ GTQVTVSSAAAGGGGSGGGGSGGGGSDKTHTCPPCPAPEAAGGPSVFLFPPKPKD TLMISRTPEVTCVVVDVSHEDPEVKFNWYVDGVEVHNAKTKPREEQYNSTYRVV SVLTVLHQDWLNGKEYKCKVSNKALGAPIEKTISKAKGQPREPQVYTLPPSRDEL TKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 64). In some embodiments, the first polypeptide consists of SEQ ID NO: 64. In some embodiments, the second polypeptide comprises SEQ ID NO: 64. In some embodiments, the second polypeptide consists of SEQ ID NO: 64. In some embodiments, the agent comprises a dimer of SEQ ID NO: 64. In some embodiments, the agent consists of a dimer of SEQ ID NO: 64. In some embodiments, the first polypeptide, the second polypeptide or both comprises or consists of a sequence with at least 70, 75, 80, 85, 90, 93, 95, 97, or 99% homology to SEQ ID NO: 64. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 64. Each possibility represents a separate embodiment of the invention. In some embodiments, the sequence with homology comprises the CDRs of SEQ ID NO: 29. In some embodiments, the sequence with homolog comprises the P to G, L to A and L to A mutations of the Fc domain. In some embodiments, the sequence with homology comprises G244, A149 and A150 of SEQ ID NO: 64.

In some embodiments, an agent comprising SEQ ID NO: 64 or a homolog thereof is not a CD28 antagonist. In some embodiments, an agent consisting of a dimer of SEQ ID NO: 64 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASINSMAWYRQAPGSQRELVAAISNARE VYYADSVKGRFTISRDNAKTTVYLQMNSLRPEDTAVYYCVVDVYFQEYWYWGQ GTQVTVSSAAAGGGGSGGGGSGGGGSDKTHTCPPCPAPEAAGGPSVFLFPPKPKD TLMISRTPEVTCVVVDVSHEDPEVKFNWYVDGVEVHNAKTKPREEQYNSTYRVV SVLTVLHQDWLNGKEYKCKVSNKALGAPIEKTISKAKGQPREPQVYTLPPSRDEL TKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 65). In some embodiments, the first polypeptide consists of SEQ ID NO: 65. In some embodiments, the second polypeptide comprises SEQ ID NO: 65. In some embodiments, the second polypeptide consists of SEQ ID NO: 65. In some embodiments, the agent comprises a dimer of SEQ ID NO: 65. In some embodiments, the agent consists of a dimer of SEQ ID NO: 65. In some embodiments, the first polypeptide, the second polypeptide or both comprises or consists of a sequence with at least 70, 75, 80, 85, 90, 93, 95, 97, or 99% homology to SEQ ID NO: 65. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 65. Each possibility represents a separate embodiment of the invention. In some embodiments, the sequence with homology comprises the CDRs of SEQ ID NO: 30. In some embodiments, the sequence with homolog comprises the P to G, L to A and L to A mutations of the Fc domain. In some embodiments, the sequence with homology comprises G244, A149 and A150 of SEQ ID NO: 65.

In some embodiments, an agent comprising SEQ ID NO: 65 or a homolog thereof is not a CD28 antagonist. In some embodiments, an agent consisting of a dimer of SEQ ID NO: 65 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASINTMAWYRQAPGSQRELVAAINSISR TYYADSVKGRFTISRDNAKTTVYLQMNSLRPEDTAVYYCVVDVTKEDYWYWGQ GTQVTVSSAAAGGGGSGGGGSGGGGSDKTHTCPPCPAPEAAGGPSVFLFPPKPKD TLMISRTPEVTCVVVDVSHEDPEVKFNWYVDGVEVHNAKTKPREEQYNSTYRVV SVLTVLHQDWLNGKEYKCKVSNKALGAPIEKTISKAKGQPREPQVYTLPPSRDEL TKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 66). In some embodiments, the first polypeptide consists of SEQ ID NO: 66. In some embodiments, the second polypeptide comprises SEQ ID NO: 66. In some embodiments, the second polypeptide consists of SEQ ID NO: 66. In some embodiments, the agent comprises a dimer of SEQ ID NO: 66. In some embodiments, the agent consists of a dimer of SEQ ID NO: 66. In some embodiments, the first polypeptide, the second polypeptide or both comprises or consists of a sequence with at least 70, 75, 80, 85, 90, 93, 95, 97, or 99% homology to SEQ ID NO: 66. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 66. Each possibility represents a separate embodiment of the invention. In some embodiments, the sequence with homology comprises the CDRs of SEQ ID NO: 31. In some embodiments, the sequence with homolog comprises the P to G, L to A and L to A mutations of the Fc domain. In some embodiments, the sequence with homology comprises G244, A149 and A150 of SEQ ID NO: 66.

In some embodiments, an agent comprising SEQ ID NO: 66 or a homolog thereof is not a CD28 antagonist. In some embodiments, an agent consisting of a dimer of SEQ ID NO: 66 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASIKTMAWYRQAPGSQRELVTAIASDN RKYYADSVKGRFTISRDNAKTTVYLQMNSLRPEDTAVYYCVVDVTKEDYWYWG QGTQVTVSSAAAGGGGSGGGGSGGGGSDKTHTCPPCPAPEAAGGPSVFLFPPKPK DTLMISRTPEVTCVVVDVSHEDPEVKFNWYVDGVEVHNAKTKPREEQYNSTYRV VSVLTVLHQDWLNGKEYKCKVSNKALGAPIEKTISKAKGQPREPQVYTLPPSRDE LTKNQVSLTCLVKGFYPSDIAVEWESNGQPENNYKTTPPVLDSDGSFFLYSKLTVD KSRWQQGNVFSCSVMHEALHNHYTQKSLSLSPGK (SEQ ID NO: 67). In some embodiments, the first polypeptide consists of SEQ ID NO: 67. In some embodiments, the second polypeptide comprises SEQ ID NO: 67. In some embodiments, the second polypeptide consists of SEQ ID NO: 67. In some embodiments, the agent comprises a dimer of SEQ ID NO: 67. In some embodiments, the agent consists of a dimer of SEQ ID NO: 67. In some embodiments, the first polypeptide, the second polypeptide or both comprises or consists of a sequence with at least 70, 75, 80, 85, 90, 93, 95, 97, or 99% homology to SEQ ID NO: 67. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 67. Each possibility represents a separate embodiment of the invention. In some embodiments, the sequence with homology comprises the CDRs of SEQ ID NO: 32. In some embodiments, the sequence with homolog comprises the P to G, L to A and L to A mutations of the Fc domain. In some embodiments, the sequence with homology comprises G244, A149 and A150 of SEQ ID NO: 67.

In some embodiments, an agent comprising SEQ ID NO: 67 or a homolog thereof is not a CD28 antagonist. In some embodiments, an agent consisting of a dimer of SEQ ID NO: 67 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASIRTMAWYRQAPGSQRELVAAISSGRE VYYADSVKGRFTISRDNAKTTVYLQMNSLRPEDTAVYYCVVDMYWQDYWWWG QGTQVTVSSAAAGGGGSGGGGSGGGGSDKTHTCPPCPAPEAAGGPSVFLFPPKPK DTLMISRTPEVTCVVVDVSHEDPEVKFNWYVDGVEVHNAKTKPREEQYNSTYRV VSVLTVLHQDWLNGKEYKCKVSNKALGAPIEKTISKAKGQPREPQVYTLPPSRDE LTKNQVSLTCLVKGFYPSDIAVEWESNGQPENNYKTTPPVLDSDGSFFLYSKLTVD KSRWQQGNVFSCSVMHEALHNHYTQKSLSLSPGK (SEQ ID NO: 68). In some embodiments, the first polypeptide consists of SEQ ID NO: 68. In some embodiments, the second polypeptide comprises SEQ ID NO: 68. In some embodiments, the second polypeptide consists of SEQ ID NO: 68. In some embodiments, the agent comprises a dimer of SEQ ID NO: 68. In some embodiments, the agent consists of a dimer of SEQ ID NO: 68. In some embodiments, the first polypeptide, the second polypeptide or both comprises or consists of a sequence with at least 70, 75, 80, 85, 90, 93, 95, 97, or 99% homology to SEQ ID NO: 68. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 68. Each possibility represents a separate embodiment of the invention. In some embodiments, the sequence with homology comprises the CDRs of SEQ ID NO: 33. In some embodiments, the sequence with homolog comprises the P to G, L to A and L to A mutations of the Fc domain. In some embodiments, the sequence with homology comprises G244, A149 and A150 of SEQ ID NO: 68.

In some embodiments, an agent comprising SEQ ID NO: 68 or a homolog thereof is not a CD28 antagonist. In some embodiments, an agent consisting of a dimer of SEQ ID NO: 68 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASINSMGWYRQAPGSQRELVAAISDRSE KYYADSVKGRFTISRDNAKTTVYLQMNSLRPEDTAVYYCVVDHHHSDWWTWGQ GTQVTVSSAAAGGGGSGGGGSGGGGSDKTHTCPPCPAPEAAGGPSVFLFPPKPKD TLMISRTPEVTCVVVDVSHEDPEVKFNWYVDGVEVHNAKTKPREEQYNSTYRVV SVLTVLHQDWLNGKEYKCKVSNKALGAPIEKTISKAKGQPREPQVYTLPPSRDEL TKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 69). In some embodiments, the first polypeptide consists of SEQ ID NO: 69. In some embodiments, the second polypeptide comprises SEQ ID NO: 69. In some embodiments, the second polypeptide consists of SEQ ID NO: 69. In some embodiments, the agent comprises a dimer of SEQ ID NO: 69. In some embodiments, the agent consists of a dimer of SEQ ID NO: 69. In some embodiments, the first polypeptide, the second polypeptide or both comprises or consists of a sequence with at least 70, 75, 80, 85, 90, 93, 95, 97, or 99% homology to SEQ ID NO: 69. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 69. Each possibility represents a separate embodiment of the invention. In some embodiments, the sequence with homology comprises the CDRs of SEQ ID NO: 34. In some embodiments, the sequence with homolog comprises the P to G, L to A and L to A mutations of the Fc domain. In some embodiments, the sequence with homology comprises G244, A149 and A150 of SEQ ID NO: 69.

In some embodiments, an agent comprising SEQ ID NO: 69 or a homolog thereof is not a CD28 antagonist. In some embodiments, an agent consisting of a dimer of SEQ ID NO: 69 or a homolog thereof is not a CD28 antagonist. In some embodiments, a CD28 antagonist is a substantial antagonist.

In some embodiments, the first polypeptide comprises EVQLVESGGGLVQAGESLRLSCAASGSIASIKTMAWYRQAPGSQRELVTAIASDN RKYYADSVKGRFTISRDNAKTTVYLQMNSLRPEDTAVYYCVVDVTKEDYWYWG QGTQVTVSSGGGGSGGGGSGGGGSGGGGSGGGGSESKYGPPCPPCPAPEFEGGPS VFLFPPKPKDTLMISRTPEVTCVVVDVSQEDPEVQFNWYVDGVEVHNAKTKPREE QFNSTYRVVSVLTVLHQDWLNGKEYKCKVSNKGLPSSIEKTISKAKGQPREPQVY TLPPSQEEMTKNQVSLTCLVKGFYPSDIAVEWESNGQPENNYKTTPPVLDSDGSFF LYSRLTVDKSRWQEGNVFSCSVMHEALHNHYTQKSLSLSLGK (SEQ ID NO:93). In some embodiments, the first polypeptide consists of SEQ ID NO: 93. In some embodiments, the second polypeptide comprises SEQ ID NO: 93. In some embodiments, the second polypeptide consists of SEQ ID NO: 93. In some embodiments, the agent comprises a dimer of SEQ ID NO: 93. In some embodiments, the agent consists of a dimer of SEQ ID NO: 93. In some embodiments, the first polypeptide, the second polypeptide or both comprises or consists of a sequence with at least 70, 75, 80, 85, 90, 93, 95, 97, or 99% homology to SEQ ID NO: 93. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 93. Each possibility represents a separate embodiment of the invention. In some embodiments, the sequence with homology comprises the CDRs of SEQ ID NO: 32. In some embodiments, the sequence with homology comprises the CDRs of SEQ ID NO: 8, SEQ ID NO: 16 and SEQ ID NO: 10. It will be understood by a skilled artisan that amino acids 1-115 of SEQ ID NO: X can be replaced with any of SEQ ID NO: 70-74.

In some embodiments, the first polypeptide comprises EVQLVESGGGLVQAGESLRLSCAASGSIASIRTMAWYRQAPGSQRELVAAISSGRE VYYADSVKGRFTISRDNAKTTVYLQMNSLRPEDTAVYYCVVDMYWQDYWWWG QGTQVTVSSGGGGSGGGGSGGGGSGGGGSGGGGSESKYGPPCPPCPAPEFEGGPS VFLFPPKPKDTLMISRTPEVTCVVVDVSQEDPEVQFNWYVDGVEVHNAKTKPREE QFNSTYRVVSVLTVLHQDWLNGKEYKCKVSNKGLPSSIEKTISKAKGQPREPQVY TLPPSQEEMTKNQVSLTCLVKGFYPSDIAVEWESNGQPENNYKTTPPVLDSDGSFF LYSRLTVDKSRWQEGNVFSCSVMHEALHNHYTQKSLSLSLGK (SEQ ID NO:94). In some embodiments, the first polypeptide consists of SEQ ID NO: 94. In some embodiments, the second polypeptide comprises SEQ ID NO: 94. In some embodiments, the second polypeptide consists of SEQ ID NO: 94. In some embodiments, the agent comprises a dimer of SEQ ID NO: 94. In some embodiments, the agent consists of a dimer of SEQ ID NO: 93. In some embodiments, the first polypeptide, the second polypeptide or both comprises or consists of a sequence with at least 70, 75, 80, 85, 90, 93, 95, 97, or 99% homology to SEQ ID NO: 94. Each possibility represents a separate embodiment of the invention. In some embodiments, the agent comprises or consists of a dimer of a polypeptide with at least 70, 75, 80, 85, 90, 93, 95, 97, or 99% homology to SEQ ID NO: 94. Each possibility represents a separate embodiment of the invention. In some embodiments, the sequence with homology comprises the CDRs of SEQ ID NO: 33. In some embodiments, the sequence with homology comprises the CDRs of SEQ ID NO: 17, SEQ ID NO: 18 and SEQ ID NO: 19. It will be understood by a skilled artisan that amino acids 1-115 of SEQ ID NO: X can be replaced with any of SEQ ID NO: 75-78.

In some embodiments, the agent does not modulate CD28 function and/or signaling. In some embodiments, the agent does not degrade mCD28. In some embodiments, the agent does not lead to or facilitate mCD28 degradation. In some embodiments, the signaling is mCD28-mediated immune cell activation. In some embodiments, the agent does not inhibit immune cell activation. In some embodiments, the agent does not induce CD28 receptor internalization or recycling. Co-stimulation via mCD28 is essential for immune activation of T-cells. Proteolytic cleavage removed the ligand-binding domain in the extracellular region of CD28 from the transmembrane and cytoplasmic portions of the protein which remain in the membrane. Thus, cleaved CD28 cannot signal and cannot contribute to T cell activation. Thus, an agent that blocks cleavage, and is also an antagonist does not allow for mCD28 activation. Similarly, an agent that blocks cleavage, but is also an agonist could induce aberrant T-cell activation, and potentially an autoimmune response. In some embodiments, the agent is not anti-CD28 antibody MAB342. In some embodiments, the agent is not anti-CD28 antibody clone #37407.

In some embodiments, the agent does not reduce surface levels of mCD28 on an immune cell. In some embodiments, the immune cell is a T cell. In some embodiments, the agent reduces surface levels of mCD28 by less than 50, 40, 30, 25, 20, 15, 10, 7, 5, 3, 2 or 1%. Each possibility represents a separate embodiment of the invention.

In some embodiments, the binding of the agent to a cell does not kill the cell. In some embodiments, the binding of the agent to a cell does not lead to death of the cell. In some embodiments the agent does not induce antibody dependent cell-mediated cytotoxicity (ADCC). In some embodiments, the agent does not induce complement-dependent cytotoxicity (CDC). In some embodiments, the agent does not induce ADCC and/or CDC.

### Single Chain Agents

In some embodiments, the agent comprises a single polypeptide. In some embodiments, the agent is a single chain agent. In some embodiments, the agent consists of a single polypeptide. In some embodiments, the single polypeptide comprises the first sdAb and the second sdAb. In some embodiments, the first sdAb is N-terminal to the second sdAb. In some embodiments, the second sdAb is N-terminal to the first sdAb. In some embodiments, the first sdAb is C-terminal to the second sdAb. In some embodiments, the second sdAb is C-terminal to the first sdAb. In some embodiments, the N-terminus of the single polypeptide is an sdAb. In some embodiments, the C-terminus of the single polypeptide is an sdAb. In some embodiments, the N-terminal domain, the C-terminal domain or both is an sdAb.

In some embodiments, two sdAbs are separated by an amino acid linker. In some embodiments, the first sdAb and the second sdAb are separated by a linker.

In some embodiments, the linker is a short linker. In some embodiments, the short linker comprises fewer than 10 amino acids. In some embodiments, the short linker comprises fewer than 13 amino acids. In some embodiments, the short linker comprises 10 or fewer amino acids. In some embodiments, the linker comprises 13 or fewer amino acids. In some embodiments, the short linker comprises at most 5, 6, 7, 8, 9, 10, 11, 12 or 13 amino acids. Each possibility represents a separate embodiment of the invention. In some embodiments, the short linker comprises 5 or fewer amino acids. In some embodiments, the short linker comprises 8 or fewer amino acids. In some embodiments, the short linker comprises at most 5 amino acids. In some embodiments, the short linker comprises at most 8 amino acids. In some embodiments, the short linker comprises at most 9 amino acids. In some embodiments, the short linker comprises at most 12 amino acids. In some embodiments, the short linker is a flexible linker. In some embodiments, the short linker is a GGGGS (SEQ ID NO: 118) linker. In some embodiments, the short linker comprises 1 GGGGS. In some embodiment, the short linker comprises (GGGGS)n wherein n is an integer. In some embodiments, the short linker comprises AAAGGGGS (SEQ ID NO: 119). In some embodiments, n is 1 or 2. In some embodiments, n is 1. In some embodiments, the short linker consists of AAAGGGGS (SEQ ID NO: 119).

In some embodiments, the single polypeptide comprises the sequence EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSSAAAGGGGSEVQLVESGGGLVQAGESLRLSCAASGSIASINAMGWY RQAPGSQRELVAAISGGGDTYYADSVKGRFTISRDNAKTTVYLQMNSLRPEDTAV YYCVVDLYGSDYWDWGQGTQVTVSS (SEQ ID NO: 103). In some embodiments, the single polypeptide consists of SEQ ID NO: 103. In some embodiments, the agent comprises SEQ ID NO: 103. In some embodiments, the agent consists of SEQ ID NO: 103. In some embodiments, the single polypeptide or the agent comprises or consists of a sequence with at least 70, 75, 80, 85, 90, 93, 95, 97, or 99% homology to SEQ ID NO: 103. Each possibility represents a separate embodiment of the invention. In some embodiments, the sequence with homology comprises the CDRs of SEQ ID NO: 40. In some embodiments, the sequence with homology comprises other sdAbs of the invention. In some embodiments, the other sdAbs of the invention are in place of 2A1.

In some embodiments, the single chain agent with a short linker is not a CD28 antagonist. In some embodiments, an agent comprising SEQ ID NO: 103 is not a CD28 antagonist. In some embodiments, an agent consisting of SEQ ID NO: 103 is not a CD28 antagonist. In some embodiments, an agonist is a substantial antagonist.

In some embodiments, the linker is a long linker. In some embodiments, the long linker comprises 10 or more amino acids. In some embodiments, the long linker comprises 13 or more amino acids. In some embodiments, the long linker comprises at least 10 amino acids. In some embodiments, the linker comprises at least 10 amino acids. In some embodiments, the long linker comprises at least 10, 11, 12, 13, 14, 15, 16, 17, 18, 19, 20, 21, 22, 23, 24, 25, 26, 27, or 28 amino acids. Each possibility represents a separate embodiment of the invention. In some embodiments, the long linker comprises 15 or more amino acids. In some embodiments, the long linker comprises 18 or more amino acids. In some embodiments, the long linker comprises at least 15 amino acids. In some embodiments, the long linker comprises at least 18 amino acids. In some embodiments, the long linker comprises at least 20 amino acids. In some embodiments, the long linker comprises at least 23 amino acids. In some embodiments, the long linker comprises 20 or more amino acids. In some embodiments, the long linker comprises 23 or more amino acids. In some embodiments, the long linker is a flexible linker. In some embodiment, the long linker comprises (GGGGS)n wherein n is an integer. In some embodiments, the long linker comprises AAA(GGGGS)n wherein n is an integer. In some embodiments, n is 2, 3, 4, 5, 6, 7, 8, 9 or 10. Each possibility represents a separate embodiment of the invention. In some embodiments, n is 2. In some embodiments, n is 3. In some embodiments, n is 4. In some embodiments, the long linker comprises or consists of AAAGGGGSGGGGS (SEQ ID NO: 46). In some embodiments, the long linker comprises or consists of AAAGGGGSGGGGSGGGGSGGGGS (SEQ ID NO: 109).

In some embodiments, the single polypeptide comprises the sequence EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSSAAAGGGGSGGGGSEVQLVESGGGLVQAGESLRLSCAASGSIASINA MGWYRQAPGSQRELVAAISGGGDTYYADSVKGRFTISRDNAKTTVYLQMNSLRP EDTAVYYCVVDLYGSDYWDWGQGTQVTVSS (SEQ ID NO: 104). In some embodiments, the single polypeptide consists of SEQ ID NO: 104. In some embodiments, the agent comprises SEQ ID NO: 104. In some embodiments, the agent consists of SEQ ID NO: 104. In some embodiments, the single polypeptide or the agent comprises or consists of a sequence with at least 70, 75, 80, 85, 90, 93, 95, 97, or 99% homology to SEQ ID NO: 104. Each possibility represents a separate embodiment of the invention. In some embodiments, the sequence with homology comprises the CDRs of SEQ ID NO: 40. In some embodiments, the sequence with homology is an sdAb of the invention. In some embodiments, the sdAb of the invention is in place of 2A1.

In some embodiments, the single chain agent with a long linker is a CD28 antagonist. In some embodiments, an agent comprising SEQ ID NO: 104 is a CD28 antagonist. In some embodiments, an agent consisting of SEQ ID NO: 104 is a CD28 antagonist. In some embodiments, an agonist is a substantial antagonist.

In some embodiments, the single polypeptide comprises the sequence

(SEQ ID NO: 105)
EVQLVESGGGLVQAGESLRLSCAASGSIASINAMGWYRQAPGSQRELVA
AISGGGDTYYADSVKGRFTISRDNAKTTVYLQMNSLRPEDTAVYYCVVD
LYGSDYWDWGQGTQVTVSSAAAGGGGSGGGGSGGGGSGGGGSEVQLVES
GGGLVQAGESLRLSCAASGSIASINAMGWYRQAPGSQRELVAAISGGGD
TYYADSVKGRFTISRDNAKTTVYLQMNSLRPEDTAVYYCVVDLYGSDYW
DWGQGTQVTVSS.

In some embodiments, the single polypeptide consists of SEQ ID NO: 105. In some embodiments, the agent comprises SEQ ID NO: 105. In some embodiments, the agent consists of SEQ ID NO: 105. In some embodiments, the single polypeptide or the agent comprises or consists of a sequence with at least 70, 75, 80, 85, 90, 93, 95, 97, or 99% homology to SEQ ID NO: 105. Each possibility represents a separate embodiment of the invention. In some embodiments, the sequence with homology comprises the CDRs of SEQ ID NO: 40. In some embodiments, the sequence with homology is an sdAb of the invention. In some embodiments, the sdAb of the invention is in place of 2A1.

In some embodiments, an agent comprising SEQ ID NO: 105 is a CD28 antagonist. In some embodiments, an agent consisting of SEQ ID NO: 105 is a CD28 antagonist. In some embodiments, an agonist is a substantial antagonist.

In some embodiments, the linker comprises a net neutral charge. In some embodiments, the linker consists of alanine (A), glycine (G) and serine (S) residues. In some embodiments, the linker comprises only alanine, glycine and serine residues. In some embodiments, the linker is devoid of charged amino acids.

In some embodiments, the linker is a charged linker. In some embodiments, the linker comprises a net charge. In some embodiments, the charged linker comprises a net positive charge. In some embodiments, the charged linker comprises a net positive charge. In some embodiments, the positively charged linker comprises at least one positively charged amino acid. In some embodiments, a positively charged amino acid is lysine (K), arginine (R) or histidine (H). In some embodiments, the charged linker comprises a net negative charge. In some embodiments, the negatively charged linker comprises at least one negatively charged amino acid. In some embodiments, a negatively charged amino acid is glutamic acid (E) or aspartic acid (D). In some embodiments, a charged linker comprises at least one charged amino acid. In some embodiments, a charged amino acid is K, R, H, E or D. In some embodiment, the charged linker comprises (GGGXS)n wherein n is an integer. In some embodiments, the charged linker comprises AAA(GGGXS)n wherein n is an integer. In some embodiments, X is a charged amino acid. In some embodiment, X is a positively charged amino acid. In some embodiment, X is a negatively charged amino acid. In some embodiments, X is K. In some embodiments, X is E. In some embodiments, n is 2, 3, 4, 5, 6, 7, 8, 9 or 10. Each possibility represents a separate embodiment of the invention. In some embodiments, n is 2. In some embodiments, n is 3. In some embodiments, n is 4. In some embodiments, the charged linker comprises or consists of AAAGGGKSGGGKSGGGKSGGGKS (SEQ ID NO: 110). In some embodiments, the long linker comprises or consists of AAAGGGESGGGESGGGESGGGES (SEQ ID NO: 111).

In some embodiments, the single polypeptide comprises the sequence

(SEQ ID NO: 106)
EVQLVESGGGLVQAGESLRLSCAASGSIASINAMGWYRQAPGSQRELVA
AISGGGDTYYADSVKGRFTISRDNAKTTVYLQMNSLRPEDTAVYYCVVD
LYGSDYWDWGQGTQVTVSSAAAGGGKSGGGKSGGGKSGGGKSEVQLVES
GGGLVQAGESLRLSCAASGSIASINAMGWYRQAPGSQRELVAAISGGGD
TYYADSVKGRFTISRDNAKTTVYLQMNSLRPEDTAVYYCVVDLYGSDYW
DWGQGTQVTVSS.

In some embodiments, the single polypeptide consists of SEQ ID NO: 106. In some embodiments, the agent comprises SEQ ID NO: 106. In some embodiments, the agent consists of SEQ ID NO: 106. In some embodiments, the single polypeptide or the agent comprises or consists of a sequence with at least 70, 75, 80, 85, 90, 93, 95, 97, or 99% homology to SEQ ID NO: 106. Each possibility represents a separate embodiment of the invention. In some embodiments, the sequence with homology comprises the CDRs of SEQ ID NO: 40. In some embodiments, the sequence with homology is an sdAb of the invention. In some embodiments, the sdAb of the invention is in place of 2A1.

In some embodiments, the single chain agent with a charged linker is a CD28 antagonist. In some embodiments, the single chain agent with a positively charged linker is a CD28 antagonist. In some embodiments, an agent comprising SEQ ID NO: 106 is a CD28 antagonist. In some embodiments, an agent consisting of SEQ ID NO: 106 is a CD28 antagonist. In some embodiments, an agonist is a substantial antagonist.

In some embodiments, the single polypeptide comprises the sequence

(SEQ ID NO: 107)
EVQLVESGGGLVQAGESLRLSCAASGSIASINAMGWYRQAPGSQRELVA
AISGGGDTYYADSVKGRFTISRDNAKTTVYLQMNSLRPEDTAVYYCVVD
LYGSDYWDWGQGTQVTVSSAAAGGGESGGGESGGGESGGGESEVQLVES
GGGLVQAGESLRLSCAASGSIASINAMGWYRQAPGSQRELVAAISGGGD
TYYADSVKGRFTISRDNAKTTVYLQMNSLRPEDTAVYYCVVDLYGSDYW
DWGQGTQVTVSS.

In some embodiments, the single polypeptide consists of SEQ ID NO: 107. In some embodiments, the agent comprises SEQ ID NO: 107. In some embodiments, the agent consists of SEQ ID NO: 107. In some embodiments, the single polypeptide or the agent comprises or consists of a sequence with at least 70, 75, 80, 85, 90, 93, 95, 97, or 99% homology to SEQ ID NO: 107. Each possibility represents a separate embodiment of the invention. In some embodiments, the sequence with homology comprises the CDRs of SEQ ID NO: 40. In some embodiments, the sequence with homology is an sdAb of the invention. In some embodiments, the sdAb of the invention is in place of 2A1.

In some embodiments, the single chain agent with a negatively charged linker is a CD28 antagonist. In some embodiments, an agent comprising SEQ ID NO: 107 is a CD28 antagonist. In some embodiments, an agent consisting of SEQ ID NO: 107 is a CD28 antagonist. In some embodiments, an agonist is a substantial antagonist.

In some embodiments, the linker is a rigid linker. In some embodiments, the linker is a helical linker. In some embodiments, the rigid linker is a helical linker. In some embodiments, the rigid linker is a long linking. In some embodiments, the rigid linker comprises (EAAAK)n, wherein n is an integer. In some embodiments, the rigid linker comprises or consists of GGGGSAEAAAKEAAAKEAAAKAAAGSGGGGS (SEQ ID NO: 97). In some embodiments, the rigid linker comprises or consists of

(SEQ ID NO: 112)
AAAGGGGSAEAAAKEAAAKEAAAKAAAGSGGGGS.

In some embodiments, the single polypeptide comprises the sequence EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSSAAAGGGGSAEAAAKEAAAKEAAAKAAAGSGGGGSEVQLVESGGG LVQAGESLRLSCAASGSIASINAMGWYRQAPGSQRELVAAISGGGDTYYADSVKG RFTISRDNAKTTVYLQMNSLRPEDTAVYYCVVDLYGSDYWDWGQGTQVTVSS (SEQ ID NO: 108). In some embodiments, the single polypeptide consists of SEQ ID NO: 108. In some embodiments, the agent comprises SEQ ID NO: 108. In some embodiments, the agent consists of SEQ ID NO: 108. In some embodiments, the single polypeptide or the agent comprises or consists of a sequence with at least 70, 75, 80, 85, 90, 93, 95, 97, or 99% homology to SEQ ID NO: 108. Each possibility represents a separate embodiment of the invention. In some embodiments, the sequence with homology comprises the CDRs of SEQ ID NO: 40.

In some embodiments, the single chain agent with a rigid linker is a CD28 antagonist. In some embodiments, an agent comprising SEQ ID NO: 108 is a CD28 antagonist. In some embodiments, an agent consisting of SEQ ID NO: 108 is a CD28 antagonist. In some embodiments, an agonist is a substantial antagonist.

### Nucleic Acid Molecules

By another aspect, there is provided a nucleic acid molecule encoding a sdAb of the invention.

By another aspect, there is provided a nucleic acid molecule encoding an agent of the invention.

In some embodiments, a nucleic acid molecule is a plurality of nucleic acid molecules. In some embodiments, a first nucleic acid molecule encodes the first polypeptide. In some embodiments, the second nucleic acid molecule encodes the second polypeptide. In some embodiments, a single nucleic acid molecule encodes both the first and second polypeptide.

In some embodiments, the nucleic acid molecule comprises a coding region encoding an sdAb of the invention. In some embodiments, the nucleic acid molecule comprises a coding region encoding an agent of the invention. In some embodiments, encoding an agent comprises encoding a first polypeptide. In some embodiments, encoding an agent comprises encoding a second polypeptide. In some embodiments, the nucleic acid molecule encodes the first and the second polypeptide. In some embodiments, the first and second polypeptides are identical, and the nucleic acid molecule comprises a single coding region encoding the polypeptides. In some embodiments, the nucleic acid molecule is a plurality of nucleic acid molecules. In some embodiments, the plurality comprises a first molecule encoding the first polypeptide and a second molecule encoding the second polypeptide.

In some embodiments, the nucleic acid molecule is a vector. In sone embodiments, the vector is an expression vector. In some embodiments, the vector is a plasmid. In some embodiments, the vector is a mammalian expression vector. In some embodiments, the mammal is human. In some embodiments, the vector is for expression in human cells. In some embodiments, the vector is for expression in culture. In some embodiments, the vector is for expression in vitro. In some embodiments, the vector is for expression in vivo. Expressing of a nucleic acid molecule that encodes an agent within a cell is well known to one skilled in the art. It can be carried out by, among many methods, transfection, viral infection, or direct alteration of the cell's genome.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the nucleic acid sequence encoding an agent is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is a mammalian promoter. In some embodiments, the promoter is configured for expression in a target cell. In some embodiments, the target cell is a mammalian cell. In some embodiments, the mammal is human.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

### Compositions

By another aspect, there is provided a composition comprising a sdAb of the invention.

By another aspect, there is provided a composition comprising an agent of the invention.

By another aspect, there is provided a composition comprising a nucleic acid molecule of the invention.

In some embodiments, the composition is a pharmaceutical composition. In some embodiments, the composition is a therapeutic composition. In some embodiments, the composition comprises a therapeutically acceptable carrier, excipient or adjuvant.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition comprises a therapeutically effective amount of the sdAb. In some embodiments, the composition comprises a therapeutically effective amount of the agent. In some embodiments, the composition comprises a therapeutically effective amount of the nucleic acid molecule. As used herein, the term “therapeutically effective amount” refers to an amount of the agent effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the composition is formulated for administration to a subject. In some embodiments, the composition is formulated for systemic administration. In some embodiments, the composition is formulated for local administration. In some embodiments, local administration is administration to a site of inflammation. In some embodiments, local administration is intratumoral administration. In some embodiments, local administration is administration to a site of immune response. In some embodiments, the immune response is autoimmune response.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of an agent of the invention to a patient in need thereof. Other suitable routes of administration can include parenteral, subcutaneous, oral, intramuscular, or intraperitoneal.

### Methods of Treatment

By another aspect, there is provided a method of decreasing sCD28 levels in a subject in need thereof, the method comprising administering to the subject a sdAb of the invention, an agent of the invention or a pharmaceutical composition of the invention, thereby decreasing sCD28 levels.

By another aspect, there is provided a method of decreasing CD28 cleavage on the surface of a cell, the method comprising contacting the cell with a sdAb of the invention, an agent of the invention or a pharmaceutical composition of the invention, thereby decreasing sCD28 levels.

By another aspect, there is provided a method of treating and/or preventing a disease in a subject in need thereof, the method comprising administering to the subject a sdAb of the invention, an agent of the invention or a pharmaceutical composition of the invention, thereby treating and/or preventing a disease.

By another aspect, there is provided a method of improving an immunotherapy in a subject in need thereof, the method comprising administering to the subject a sdAb of the invention, an agent of the invention or a pharmaceutical composition of the invention, thereby improving an immunotherapy.

The ability of sCD28 reduction and in particular the blocking of CD28 cleavage has been demonstrated to be an effective treatment for cancer, a way to increase immune stimulation and an effective boost to immunotherapy. This has been demonstrated in International Patent Applications WO2019/175885 and WO2020111441, herein incorporated by reference in their entirety.

In some embodiments, the immunotherapy is PD-1 and/or PD-L1 based immunotherapy. In some embodiments, the PD-1/PD-L1 based immunotherapy comprises administering an anti-PD1 or anti-PD-L1 antibody. In some embodiments, the therapy comprises blockade of the PD-1 checkpoint. In some embodiments, the immunotherapy comprises administering allogenic, syngenic or autologous immune cells to the subject. In some embodiments, the immune cells are T cells. In some embodiments, the subject in need of immunotherapy suffers from cancer.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, the subject is a mammal. In some embodiments, the subject is a human. In some embodiments, the subject suffers from a disease. In some embodiments, the subject is in need thereof. In some embodiments, the subject is in need of immunotherapy. In some embodiments, the subject is being treated by immunotherapy.

In some embodiments, the subject's blood comprises elevated levels of sCD28. In some embodiments, the subject's blood before the decreasing comprises elevated levels of sCD28. In some embodiments, the levels are elevated above those of healthy subjects. In some embodiments, the subject's sCD28 levels are elevated by at least 5%, 10%, 15%, 20%, 25%, 30%, 40%, 50%, 60%, 70%, 80%, 90%, 100%, 150%, 200%, 250%, 300%, 350%, 400%, 500%, 600%, 700%, 800%, 900%, or 1000% above healthy subject levels. Each possibility represents a separate embodiment of the invention. In some embodiments, the levels are elevated above 5, 6, 7, 8, 9, 10, 12, 14, 15, 16, 18, 20, 25, 30, 35, 40, 45 or 50 ng/ml of blood. Each possibility represents a separate embodiment of the invention. In some embodiments, the levels are elevated above 5 ng/ml. In some embodiments, the levels are elevated above 10 ng/ml. In some embodiments, the levels are elevated above 20 ng/ml. In some embodiments, the subject's blood comprises at least 5, 6, 7, 8, 9, 10, 12, 14, 15, 16, 18, 20, 25, 30, 35, 40, 45 or 50 ng sCD28 per ml of blood. Each possibility represents a separate embodiment of the invention. In some embodiments, the subject's blood prior to the decreasing comprises at least 5, 6, 7, 8, 9, 10, 12, 14, 15, 16, 18, 20, 25, 30, 35, 40, 45 or 50 ng sCD28 per ml of blood. Each possibility represents a separate embodiment of the invention. In some embodiments, the subject's blood comprises at least 5 ng/ml sCD28. In some embodiments, the subject's blood comprises at least 10 ng/ml sCD28. In some embodiments, the subject's blood comprises at least 20 ng/ml sCD28. In some embodiments, the subject's blood prior to the decreasing comprises at least 5 ng/ml sCD28. In some embodiments, the subject's blood prior to the decreasing comprises at least 10 ng/ml sCD28. In some embodiments, the subject's blood prior to the decreasing comprises at least 20 ng/ml sCD28.

In some embodiments, the subject comprises increasing levels of sCD28. In some embodiments, increasing levels are increasing levels in blood. In some embodiments, increasing is from a first time point to a second time point. In some embodiments, increasing is from before treatment with an immunotherapy to after treatment. In some embodiments, increasing is increasing as the disease develops. It has been shown in International Patent Application WO2021/111442, the contents of which are hereby incorporated by reference in their entirety, that sCD28 levels can increase during cancer progression and during immunotherapy and that cleavage blocking agents can be used to treat such cancers.

In some embodiments, the subject suffers from cancer. In some embodiments, the cancer can be treated by immunotherapy. In some embodiments, the cancer is a cancer that can be treated with PD-1/PD-L1 therapy. In some embodiments, the subject has undergone PD-1/PD-L1 therapy. In some embodiments, the subject is a non-responder to PD-1/PD-L1 therapy. In some embodiments, the subject is naïve to PD-1/PD-L1 therapy. In some embodiments, the methods of the invention are performed together with PD-1/PD-L1 therapy. In some embodiments, the methods of the invention are performed before PD-1/PD-L1 therapy.

In some embodiments, the method further comprises administering another immunotherapy to the subject. In some embodiments, the method further comprises administering a PD-1 and/or PD-L1 based immunotherapy. In some embodiments, the another immunotherapy is a checkpoint inhibitor. In some embodiments, the checkpoint inhibitor is a PD-1 and/or PD-L1 inhibitor. In some embodiments, the checkpoint inhibitor is a CTLA-4 inhibitor. In some embodiments, the another immunotherapy is a chimeric antigen receptor (CAR) based immunotherapy. In some embodiments, the CAR is a CAR-T. In some embodiments, the CAR is a CAR-NK. In some embodiments, the another immunotherapy is a cancer vaccine.

As used herein, the terms “CAR-T cell” and “CAR-NK cell” refer to an engineered receptor which has specificity for at least one protein of interest (for example an immunogenic protein with increased expression following treatment with an epigenetic modifying agent) and is grafted onto an immune effector cell (a T cell or NK cell). In some embodiments, the CAR-T cell has the specificity of a monoclonal antibody grafted onto a T-cell. In some embodiments, the CAR-NK cell has the specificity of a monoclonal antibody grafted onto a NK-cell. In some embodiments, the T cell is selected from a cytotoxic T lymphocyte and a regulatory T cell.

CAR-T and CAR-NK cells and their vectors are well known in the art. Such cells target and are cytotoxic to the protein for which the receptor binds. In some embodiments, a CAR-T or CAR-NK cell targets at least one viral protein. In some embodiments, a CAR-T or CAR-NK cell targets a plurality of viral proteins. In some embodiments, a CAR-T or CAR-NK cell targets a viral protein with increased expression due to contact with an epigenetic modifying agent.

Construction of CAR-T cells is well known in the art. In one non-limiting example, a monoclonal antibody to a viral protein can be made and then a vector coding for the antibody will be constructed. The vector will also comprise a costimulatory signal region. In some embodiments, the costimulatory signal region comprises the intracellular domain of a known T cell or NK cell stimulatory molecule. In some embodiments, the intracellular domain is selected from at least one of the following: CD3Z. CD27, CD28, 4-1BB, OX40, CD30, CD40, PD-1, ICOS, lymphocyte function-associated antigen-1 (LFA-1), CD2, CD 7, LIGHT, NKG2C, B7-H3, and a ligand that specifically binds with CD83. In some embodiments, the vector also comprises a CD3Z signaling domain. This vector is then transfected, for example by lentiviral infection, into a T-cell.

In some embodiments, the cancer is a cancer with elevated sCD28 levels. In some embodiments, the cancer is in a subject with elevated sCD28 levels. In some embodiments, the cancer comprises high sCD28 levels. In some embodiments, the cancer is in a subject with high sCD28 levels. In some embodiments, elevated and/or high sCD28 levels are levels at and/or above 5, 6, 7, 8, 9, 10, 12, 14, 15, 17, 20, 25, 30, 35, 40, 50, 60, 70, 80, 90 or 100 ng/ml. Each possibility represents a separate embodiment of the invention. In some embodiments, the cancer comprises high sCD28 levels. In some embodiments, elevated and/or high sCD28 levels are levels at and/or above 5, 10, 15, 20, 25, 30, 35, 40, 45, 50, 55, 60, 65, 70, 75, 80, 85, 90, 95, 100, 150, 200, 300, 400, 500, 600, 700, 800, 900, or 1000% of the levels in a healthy subject. Each possibility represents a separate embodiment of the invention. In some embodiments, the cancer is not breast cancer. In some embodiments, the cancer is selected from melanoma, head and neck, non-small cell lung cancer, ovarian, kidney, gastric and colorectal. In some embodiments, the cancer is selected from melanoma, head and neck, non-small cell lung cancer, ovarian, and colorectal. In some embodiments, the cancer is melanoma, head and neck, non-small cell lung cancer, ovarian, kidney, gastric or colorectal. Each possibility represents a separate embodiment of the invention. Examples of cancer include, but are not limited to brain cancer, oral cancer, head and neck cancer, esophageal cancer, lung cancer, skin cancer, liver cancer, pancreatic cancer, bladder cancer, renal cancer, blood cancer, bladder cancer, bone cancer, breast cancer, thyroid cancer, cervical cancer, ovarian cancer, testicular cancer, retinoblastoma, gastric cancer, colorectal cancer, and uterine cancer.

In some embodiments, the disease is a proliferative disease. In some embodiments, the disease is cancer. In some embodiments, the disease is treatable by immune stimulation. In some embodiments, the disease comprises elevated sCD28 levels. In some embodiments, the disease comprises increasing levels of sCD28. In some embodiments, increasing is increasing overtime. In some embodiments, increasing is increasing during the progression of the disease. In some embodiments, the disease is an infectious disease. In some embodiments, the disease is a disease treatable by immunotherapy.

In some embodiments, the method is performed in vivo. In some embodiments, the method is performed in vitro. In some embodiments, the decreasing is performed in vivo. In some embodiments, the decreasing is performed in vitro. In some embodiments, the decreasing comprises removing blood from the subject decreasing the sCD28 levels in the removed blood and returning the blood to the subject, thereby decreasing sCD28 in the subject. Methods of dialysis and blood cleaning are well known. The invention may be practiced by in vitro sweeping away the sCD28 and then returning the blood to the subject.

In some embodiments, the agent reduces sCD28 levels by at least 5, 10, 15, 20, 25, 30, 35, 40, 45, 50, 55, 60, 65, 70, 75, 80, 85, 90, 95, 97 or 99%. Each possibility represents a separate embodiment of the invention. In some embodiments, the agent reduces sCD28 levels to that of a healthy individual. In some embodiments, the agent reduces sCD28 levels to at most 1, 2, 3, 4, 5, 10, 15, 20, 25, 30, 35, 40, 45, or 50 ng/ml. Each possibility represents a separate embodiment of the invention. In some embodiments, the agent reduces sCD28 blood levels to at most 5 ng/ml. In some embodiments, the agent reduces sCD28 blood levels to at most 10 ng/ml. In some embodiments, the agent reduces sCD28 blood levels to at most 20 ng/ml. In some embodiments, the agent reduces sCD28 levels to that of a healthy individual. In some embodiments, the agent reduces sCD28 levels to below 1, 2, 3, 4, 5, 10, 15, 20, 25, 30, 35, 40, 45, or 50 ng/ml. Each possibility represents a separate embodiment of the invention. In some embodiments, the agent reduces sCD28 levels to below 5 ng/ml. In some embodiments, the agent reduces sCD28 levels to below 10 ng/ml. In some embodiments, the agent reduces sCD28 levels to below 20 ng/ml. In some embodiments, the reducing or decreasing occurs in blood, peripheral blood or the TME of the subject. In some embodiments, the reducing or decreasing occurs in blood.

In some embodiments, sCD28 levels are as measured by ELISA. In some embodiments, the ELISA is a sandwich ELISA. In some embodiments, the ELISA is a standardized sandwich ELISA. In some embodiments, the ELISA is a Bender MedSystems ELISA. In some embodiments, the ELISA is Bender MedSystems ELISA kit BMS290. In some embodiments, the ELISA is performed with an agent of the invention.

In some embodiments, blocking CD28 shedding comprises blocking proteolytic cleavage. In some embodiments, blocking is inhibiting. In some embodiments, blocking is reducing.

As used herein, “inhibiting proteolytic cleavage” refers to any reduction in proteolytic cleavage of mCD28. In some embodiments, the inhibition is a reduction in cleavage of at least 5, 10, 15, 20, 25, 30, 35, 40, 45, 50, 55, 60, 65, 70, 75, 80, 85, 90, 95, 97, 99 or 100%. Each possibility represents a separate embodiment of the invention. In some embodiments, inhibiting proteolytic cleavage maintains levels of mCD28 on immune cells. In some embodiments, inhibiting proteolytic cleavage increases levels of mCD28 on immune cells. In some embodiments, inhibiting proteolytic cleavage maintains levels of mCD28 adequate for immune-stimulation. In some embodiments, the reduction in proteolytic cleavage is reduction in cleavage by at least one protease.

In some embodiments, the methods of the invention do not degrade or lead to degradation of mCD28. In some embodiments, the methods of the invention do not decrease mCD28 levels on immune cells. In some embodiments, the methods of the invention do not decrease mCD28-mediated immune cell activation. In some embodiments, the methods of the invention maintain mCD28 levels on immune cells in the subject. In some embodiments, the methods of the invention increase mCD28 levels on immune cells in the subject.

In some embodiments, the reduction is at least a 10, 20, 30, 40, 50, 60, 70, 80, 90, 95, or 99% reduction in sCD28. Each possibility represents a separate embodiment of the invention. In some embodiments, the reduction is in serum sCD28. In some embodiments, the reduction is in the blood levels of sCD28. In some embodiments, the reduction is in the levels of sCD28 in the tumor microenvironment (TME).

In some embodiments, the methods that increase immune activation, treat disease and/or improve immunotherapy comprise administering agents that are not antagonists. In some embodiments, the methods that increase immune activation, treat disease and/or improve immunotherapy comprise administering agents that do not substantially antagonize. In some embodiments, the disease is a disease treatable by increased immune activation.

By another aspect, there is provided a method of inhibiting ligand binding to CD28, the method comprising contacting the CD28 with a sdAb of the invention or composition of the invention, thereby inhibiting ligand binding.

By another aspect, there is provided a method of suppressing an immune response in a subject in need thereof, the method comprising administering to the subject a sdAb of the invention or a composition of the invention, thereby suppressing an immune response.

It is well known in the art that inhibition of CD28 blocks immune stimulation and can be used to treat autoimmune disease. Further, International Patent Application WO2020/183471, herein incorporated by reference in its entirety, has demonstrated that increased sCD28 levels and CD28 inhibition are therapeutically effective modalities. In some embodiments, inhibiting ligand binding comprises suppressing immune response.

In some embodiments, the agent reduces T cell activation. In some embodiments, the agent reduces T cell proliferation. In some embodiments, the agent reduces T cell clustering. In some embodiments, the agent increases anti-inflammatory cytokine secretion. Anti-inflammatory cytokines are well known in the art. Non-limiting examples of anti-inflammatory cytokines include, but are not limited to, IL-10, and TGFβ. In some embodiments, the agent decreases pro-inflammatory cytokine secretion. In some embodiments, the pro-inflammatory cytokine is IFNγ.

In some embodiments, the agent modulates CD28 function and/or signaling. In some embodiments, the agent reduces CD28 function and/or signaling. In some embodiments, the agent reduces CD28 activation. In some embodiments, the signaling is CD28-mediated immune response. In some embodiments, the agent increases or promotes immune suppression.

As used herein, the term “immune response” refers to any response taken by the body to defend itself from pathogens or abnormalities. In one embodiment, an immune response comprises a response mediated or involving an immune cell.

In one embodiment, an immune response comprises any response activating or inhibiting the immune system or mediators of the immune system. In another embodiment, activation of an immune response comprises activation of an immune cell. In another embodiment, activation of an immune cell results in the proliferation of a sub-set of immune cells. In another embodiment, activation of an immune cell results in increased secretion of an immunologic mediator by the activated cell. In another embodiment, activation of an immune cell results in the engulfment and/or destruction of a pathogen, a foreign cell, a diseased cell, a molecule derived or secreted therefrom, or any combination thereof. In another embodiment, activation of an immune cell results in the engulfment and or destruction of a neighboring cell, such as, but not limited to, a cell infected by a virus. In another embodiment, activation of an immune cell results in the engulfment and/or destruction of a host cell, a molecule derived or secreted therefrom, or any combination thereof. In another embodiment, activation of an immune cell results in activating the secretion of antibodies directed to a certain molecule, epitope, pathogen, or any combination thereof.

In some embodiments, an immune response is a cytotoxic response. As used herein, cytotoxic response refers to a response comprising activation of the complement system, leading to cell lysis and/or other damage. In some embodiments, an immune response is a humoral response, i.e., involves production and secretion of antibodies. In some embodiments, an immune response is an innate response, i.e., involves the innate immune system. In some embodiments, an immune response is an acquired immune response, i.e., involves the acquired immune response.

In some embodiments, the subject is a graft recipient or a candidate for engraftment. In some embodiment, the graft comprises solitary cells, cell suspension, an organ, or any combination thereof. In some embodiments, the graft is an autologous graft. In some embodiment, the graft is a syngeneic graft. In some embodiments, the graft is an allogenic graft. In some embodiments, the graft is a xenograft. In some embodiments, the graft is a hematopoietic graft. In some embodiments, the graft comprises hematopoietic stem cells. In some embodiments, the graft is a non-hematopoietic graft.

In some embodiments, the subject is afflicted with allergy or an allergic reaction. In some embodiments, the allergic reaction results from an infectious disease or disorder. In some embodiments, the allergic reaction is a symptom of an infectious disease or disorder. In some embodiments, the allergic reaction is independent of an infectious disease or disorder. In some embodiment, the allergic reaction is stimulated in parallel to an infectious disease or disorder.

In some embodiments, the subject is afflicted with a cytokine release syndrome (CRS). As used herein, “cytokine release syndrome” refers to a systemic inflammatory response syndrome resulting from a complication of other disease or infection. In one embodiment, CRS is induced by or results from (e.g., an adverse effect) an immunotherapy, such as a monoclonal antibody drug. In one embodiment, CRS is induced by or results from an adoptive T-cell therapy. As used herein, the terms “CRS” and “cytokine storm” are interchangeable.

In some embodiments, the subject is afflicted with an infectious disease. Non-limiting examples for infectious disease, include, but are not limited to: urinary tract infection, gastrointestinal infection, enteritis, salmonellosis, diarrhea, nontuberculous mycobacterial infections, legionnaires' disease, hospital-acquired pneumonia, skin infection, cholera, septic shock, periodontitis, infection, sinusitis, bacteremia, neonatal infections, pneumonia, endocarditis, osteomyelitis, toxic shock syndrome, scalded skin syndrome, and food poisoning.

In some embodiments, the subject is afflicted with an autoimmune disease. As used herein, the term “autoimmune disease” refers to any disease or disorder resulting from an immune response against the subject's own tissue or tissue components (e.g., cells and molecules produced or secreted by same), or to antigens that are not intrinsically harmful to the subject. In some embodiments, the subject is afflicted with a T-cell-mediated autoimmune disease. Examples of an autoimmune disease include, but are not limited to Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile myositis (JM), Kawasaki disease, Lambert-Eaton syndrome, Leukocytoclastic vasculitis, Lichen planus, Lichen sclerosis, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetesmellitus, Ulcerative colitis (UC), Undifferentiated connective tissue disease (UCTD), Uveitis, Vasculitis, Vitiligo, and Vogt-Koyanagi-Harada Disease. In some embodiments, the autoimmune disease is selected from lupus, rheumatoid arthritis, Crohn's disease, inflammatory bowel disease, Becht's disease, colitis, ulcerative colitis, diabetes, Graves' disease, and multiple sclerosis.

In some embodiments, the method further comprises administering at least one other immunosuppressive therapy. In some embodiments, an immunosuppressive therapy is an immunosuppressant. In some embodiments, an immunosuppressant is an immunosuppressive agent. In some embodiments, the immunosuppressant is sCD28 or a derivative thereof that binds ligand. In some embodiments, an immunosuppressant is a sCD28 stabilizing agent provided in International Patent Application WO2020/183471, herein incorporated by reference in its entirety. In some embodiments, the immunosuppressant is steroids. In some embodiments, the immunosuppressant is a calcineurin inhibitor. In some embodiments, the immunosuppressant is an antiproliferative agent. In some embodiments, the immunosuppressant is an mTOR inhibitor. Immunosuppressants are well known in the art and any such therapy may be employed. Examples of immunosuppressants include, but are not limited to prednisone, sirolimus, tacrolimus, cyclosporine, mycophenolate, mycophenolate sodium, azathioprine, lenalidomide, pomalidomide, methotrexate, azathioprine, and thalidomide.

In some embodiments, the autoimmune disease is an autoimmune disease with elevated sCD28 levels. In some embodiments, the autoimmune disease comprises high sCD28 levels. In some embodiments, elevated and/or high sCD28 levels are levels at and/or above 5, 6, 7, 8, 9, 10, 12, 14, 15, 17, 20, 25, 30, 35, 40, 50, 60, 70, 80, 90 or 100 ng/ml. Each possibility represents a separate embodiment of the invention. In some embodiments, the autoimmune disease comprises high sCD28 levels. In some embodiments, elevated and/or high sCD28 levels are levels at and/or above 5, 10, 15, 20, 25, 30, 35, 40, 45, 50, 55, 60, 65, 70, 75, 80, 85, 90, 95, 100, 150, 200, 300, 400, 500, 600, 700, 800, 900, or 1000% of the levels in a healthy subject. Each possibility represents a separate embodiment of the invention. In some embodiments, the autoimmune disease does not comprise elevated levels of sCD28. In some embodiments, the autoimmune disease does not comprise high levels of sCD28. In some embodiments, high and/or elevated levels are as compared to a healthy subject.

In some embodiments, the subject has elevated sCD28 levels compared to a healthy subject. In some embodiments, the subject has non-elevated sCD28 levels compared to a healthy subject. In some embodiments, the subject and the healthy subject have comparable sCD28 levels. In some embodiments, a subject having non-elevated sCD28 levels or sCD28 levels comparable to a healthy subject, has 0 to less than 5% more sCD28 than a healthy subject. In some embodiments, a subject having non-elevated sCD28 levels or sCD28 levels comparable to a healthy subject, comprises less than 5 ng/ml of sCD28.

In some embodiments, a subject having elevated sCD28 levels comprises blood sCD28 levels elevated by at least 5%, 10%, 15%, 20%, 25%, 30%, 40%, 50%, 60%, 70%, 80%, 90%, 100%, 150%, 200%, 250%, 300%, 350%, 400%, 500%, 600%, 700%, 800%, 900%, or 1,000% above healthy subject levels, or any value and range there between. Each possibility represents a separate embodiment of the invention. In some embodiments, the blood sCD28 levels are elevated by 5-25%, 10-50%, 25-75%, 50-125%, 100-250%, 200-550%, 500-750%, or 700-1,000% above healthy subject levels. In some embodiments, a subject having elevated sCD28 levels comprises levels elevated above 5, 6, 7, 8, 9, 10, 12, 14, 15, 16, 18, 20, 25, 30, 35, 40, 45 or 50 ng/ml of blood. Each possibility represents a separate embodiment of the invention. In some embodiments, the levels are elevated above 5 ng/ml. In some embodiments, the levels are elevated above 10 ng/ml.

By another aspect, there is provided a method of selecting a subject suitable for treatment by a therapeutic method of the invention, the method comprising measuring sCD28 levels in the subject and/or cancer, wherein sCD28 levels above a predetermined threshold indicates the subject is suitable for treatment by a method of the invention.

In some embodiments, the method further comprises confirming elevated sCD28 levels. In some embodiments, the method further comprises measuring sCD28 levels. In some embodiments, sCD28 levels are levels in the subject. In some embodiments, sCD28 levels are levels in the cancer. In some embodiments, sCD28 levels are levels in a sample from the subject. In some embodiments, the sample is a bodily fluid. In some embodiments, the predetermined threshold is the levels in a healthy subject. In some embodiments, the predetermined threshold is a threshold above which levels are high and/or elevated. In some embodiments, the predetermined threshold is 5 ng/ml.

By another aspect, there is provided a sdAb of the invention, an agent of the invention or a pharmaceutical composition of the invention for use in treating and/or preventing cancer.

By another aspect, there is provided a sdAb of the invention, an agent of the invention or a pharmaceutical composition of the invention for use in improving immunotherapy.

By another aspect, there is provided a sdAb of the invention or a pharmaceutical composition of the invention for use in suppressing an immune response.

### Kits

By another aspect, there is provided a kit comprising at least one sdAb of the invention or at least one agent of the invention.

In some embodiments, the kit comprises at least one composition of the invention. In some embodiments, the kit further comprises at least one immunotherapy. In some embodiments, the kit comprises a label stating the agent of the invention is for use with the immunotherapy. In some embodiments, the kit comprises a label stating the immunotherapy is for use with the agent of the invention.

### Methods of Production

By another aspect, there is provided a method for generating an agent of the invention, the method comprising:
i. obtaining a sdAb that binds to CD28 and blocks cleavage; and
ii. linking a first moiety of the sdAb to a second moiety of the sdAb via a linker to produce a dimeric agent;
   thereby producing an agent.

By another aspect, there is provided a method for generating an agent of the invention, the method comprising culturing a host cell comprising one or more vectors comprising one or more nucleic acid sequences encoding a dimeric agent, wherein the one or more nucleic acid sequences are that of a dimeric agent that was selected by:
i. obtaining a sdAb that binds to CD28 and blocks cleavage; and
ii. linking a first moiety of the sdAb to a second moiety of the sdAb via a linker to produce a dimeric agent;
thereby producing an agent.

In some embodiments, the agent is an sdAb. In some embodiments, the sdAb binds mCD28. In some embodiments, the sdAb binds mCD28 on a cell. In some embodiments, the sdAb binds mCD28 on a cell surface. In some embodiments, cleavage is by a protease. In some embodiments, the method further comprises testing an ability of the dimeric agent to block cleavage. In some embodiments, the method of selecting further comprises testing an ability of the dimeric agent to block cleavage. In some embodiments, the method further comprises selecting a dimeric agent that blocks cleavage of CD28. In some embodiments, the method of selecting further comprises selecting a dimeric agent that blocks cleavage of CD28.

In some embodiments, testing a sdAb's ability to block cleavage is by a method described hereinbelow. In some embodiments, testing a sdAb's ability to block cleavage comprises mixing of the sdAb, the protease and an extracellular domain of CD28 or a fragment thereof comprising a cleavage site. In some embodiments, the testing further comprises sequencing the extracellular domain of CD28 or a fragment thereof to check for truncation and/or cleavage. In some embodiments, the testing further comprises run the extracellular domain of CD28 or a fragment thereof on a gel that is sufficiently sensitive to measure the size change due to cleavage. In some embodiments, the testing further comprises measuring the production of sCD28 from cells expressing mCD28 in the presence of the agent and the protease.

In some embodiments, the obtained sdAb is a CD28 antagonist. In some embodiments, the obtained sdAb blocks or inhibits binding of a ligand to CD28. In some embodiments, the obtained sdAb blocks or inhibits CD28 mediated immune activation. In some embodiments, the method further comprises confirming the antagonism, blocking and/or inhibition caused by the sdAb.

In some embodiments, the method further comprises isolating and/or extracting the agent from the host cell. In some embodiments, the method further comprises isolating and/or extracting the agent from the culture media of the host cell. In some embodiments, the method further comprises purifying the agent from the host cell or the culture media of the host cell.

In some embodiments, the obtaining an agent comprises immunizing a shark or camelid with said CD28 extracellular domain or fragment thereof and collecting antibodies from said immunized organism. In some embodiments, the obtaining an agent comprises screening a library of agents for binding to a CD28 extracellular domain or fragment thereof and selecting an agent that binds.

In some embodiments, the collecting an antibody comprises extracting B cells from a spleen of the immunized shark or camelid. In some embodiments, the B cells are fused with a melanoma cell to produce a hybridoma. In some embodiments, the antibodies are collected from the culture media of the hybridoma. In some embodiments, obtaining the agent comprises immunizing an organism with the CD28 extracellular domain or fragment thereof, and collecting antibodies from the immunized organism. In some embodiments, the organism is a mouse. In some embodiments, the organism is selected from a rabbit, a mouse, a rat, a shark, a camelid, a chicken a goat and a phage. In some embodiments, the camelid is selected from a camel and a llama. In some embodiments, the collecting comprises drawing blood. In some embodiments, the collecting comprises:
a. extracting B cells from a spleen of the immunized organism;
b. fusing the extracted B cells with myeloma cells to produce a hybridoma; and
c. collecting antibodies from the hybridoma.

In some embodiments, obtaining the sdAb comprises screening a library of sdAb for binding to a CD28 extracellular domain or fragment thereof and selecting a sdAb that so binds. In some embodiments, obtaining the agent comprises screening a library of agents for binding to a CD28 extracellular domain or fragment thereof and selecting an agent that so binds. In some embodiments, obtaining the sdAb comprises screening a library of sdAbs for binding to a CD28 extracellular domain or fragment thereof and selecting a sdAb that so binds. In some embodiments, the library is a phage display library. In some embodiments, the library is an immunized library derived from splenic B cells. In some embodiments, the library is a library of VHH antibodies. In some embodiments, the library is a library of single domain or nanobodies. In some embodiments, obtaining the sdAb comprises sequencing the sdAb. In some embodiments, obtaining the sdAb comprises producing a recombinant form of the sdAb. In some embodiments, the recombinant form is produced from the sequence of the sdAb. In some embodiments, the method further comprises humanizing the sdAb. In some embodiments, obtaining the sdAb comprises sequencing the sdAb. In some embodiments, obtaining the sdAb comprises producing a recombinant form of the sdAb. In some embodiments, the recombinant form is produced from the sequence of the sdAb. In some embodiments, the method further comprises humanizing the sdAb.

In some embodiments, the method further comprises testing binding of the agent or sdAb to mCD28. In some embodiments, the mCD28 is on a cell surface. In some embodiments, the method further comprises selecting sdAbs or agents that bind to the mCD28. In some embodiments, the method further comprises testing cleavage of mCD28 on a cell surface in the presence of a protease. In some embodiments, the method further comprises selecting an agent or sdAb that blocks or inhibits cleavage of the mCD28 by the protease.

In some embodiments, a moiety is a copy of the sdAb. In some embodiments, the moiety is a single copy. In some embodiments, the moiety is the VHH. In some embodiments, the moiety is a sdAb of the invention.

In some embodiments, the method further comprises assaying mCD28 downstream signaling in the presence of the obtained dimeric agent. In some embodiments, the method further comprises selecting at least one dimeric agent that does not substantially agonizes mCD28 signaling. In some embodiments, the method further comprises selecting at least one dimeric agent that does not substantially antagonizes mCD28 signaling. In some embodiments, the method comprises selecting at least one dimeric agent that does antagonize mCD28 signaling. It will be understood by a skilled artisan that for cancer treatment agonizing CD28 signaling might not be deleterious, but that antagonizing the signaling would be counterproductive. It will be further understood that for treating autoimmune diseases or other conditions that would benefit from immune suppression antagonism would be advantageous and agonizing CD28 signaling would be counterproductive. Methods of measuring agonism and antagonism are well known in the art and further are provided hereinbelow.

By another aspect, there is provided a dimeric agent produced by a method of the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Production of recombinant 2A1 constructs—Synthetic codon-optimized genes were subcloned into relevant pcDNA3.1 expression vectors. 2A1 constructs were produced from transiently transfected ExpiCHO cells and purified by immobilized metal affinity chromatography (IMAC) for tandem constructs, MabSelect Sure Protein A for Fc chimera or Amsphere A3 for di-VHH2 2A1 construct. Protein preparations in 1×PBS pH 7.4 were analyzed by SDS-PAGE for the presence of correct chains under non-reducing conditions and by analytical size exclusion chromatography (aSEC) for the quantification of the monomeric form within the preparation.

Production of recombinant nanobodies—Synthetic codon-optimized genes were subcloned into relevant pcDNA3.1 expression vectors. Nanobodies constructs were produced from transiently transfected ExpiCHO cells and purified by immobilized metal affinity chromatography (IMAC) for tandm construct, Mabselect Sure ProteinA for Fc chimera. Protein preparations in 1×PBS pH 7.4 were analyzed by SDS-PAGE for the presence of correct chains under non-reducing conditions and by analytical size exclusion chromatography (aSEC) for the quantification of monomeric form within the preparation.

Chemical modification of parental 2A1 molecule—2A1 construct carrying a C-terminal Cysteine (2A1-1C) was incubated with a Bis-Mal-PEG11 chemical moiety (Broadpharm, Cat. No. BP-22151). Tris (2-carboxyethyl) phosphine hydrochloride (TCEP) (Sigma Aldrich, Cat. No. 75259) was added prior to the reaction to resolve dimeric content. Following the completion of the reaction, mixtures were loaded on SP cation exchange column to remove excess reagents and unreacted material. The preparations were PBS desalted using Viva-spin concentrators and analyzed by mass-spectrometry for conjugation validation.

Cytokines ELISA—Commercial ELISA kits were used for quantitation of the amount of human interferon-gamma (Biolegend, Cat. No. 430103), human interleukin 2 (Biolegend, Cat. No. 431802) and human CD28 (R&D system, Cat. No. DY342). Cell Proliferation and viability (MTT assay) was conducted to assure validity of results and performed according to manufacturer instructions (Roche, Cat. No. 11465007001).

Direct CD28 EIA—Unless discussed otherwise, Corning high binding plates or equivalent were used for screening. Each well was coated with 300 ng of human CD28-Fc chimera (R&D, Cat. No. 342-CD). Plates were blocked using 1% casein in PBS for 1 hr. at room temperature (RT). Plates were washed 4 times using PBST and incubated with investigated nanobody/agent following detection with Donkey anti human IgG FC HRP (Jackson immuno research, Cat. No. 709-035-098) for Fc chimera, Rabbit anti Camelid VHH Cocktail conjugated with HRP (GenScript, Cat. No. A02016) for tandem dimeric VHH hinge constructs, or by Mouse Anti-Human IgG4 pFc′ HRP (abeam, Cat. No. ab99817).

Cell lines and isolation of human immune cells—PBMCs were isolated from fresh blood samples of healthy donors using standard lymphocytes separation medium (MBP, Cat. No. 850494). CD3 cells were isolated from fresh blood samples of healthy donors using RossetteSEP™ Human T cells Enrichment Kit (STEMCELL, Cat. No. 15061) by negative selection method. Monocytes were isolated from fresh blood samples of healthy donors using EasySep™ Human Monocyte Enrichment Kit (STEMCELL, Cat. No. 17952) by negative selection method. All cells were grown in complete RPMI-1640 media supplemented with 10% HI-FCS and pen/strep mixture.

Transfection—CD28-FL (encoding the full-length CD28 transcript), CD80-FL (encoding the full-length CD80 transcript) and scOKT3-CD14 (encoding the single-chain FV portion of mouse anti-CD3 OKT3 clone fused to CD14 extra-cellular domain) plasmids were generated by cloning the DNA sequences into a PCDNA3.1 vector. Transfections were done using Jet Pei Transfection regent (PolyPlus Transfections). Stable transfectants were selected in G418 and/or hygromycin-containing medium.

Dendritic cell differentiation—Monocytes were cultured at a density of 1×10\hat{ }6/mL in RPMI medium with growth factors that was refreshed at day 3 and at day 6. Immature dendritic cells (iDCs) were induced by 50 ng/mL GM-CSF (R&D systems, Cat. No. 215-GM) and 20 ng/mL IL-4 (R&D systems, Cat. No. 204-IL) for 6 days. When needed the iDCs were further differentiated into mature dendritic cells by addition of 100 ng/mL LPS (Sigma, Cat. No. L4391) and 20 ng/mL interferon-gamma (R&D systems, Cat. No. 285-IF) for 48 hrs. The generated cell populations were tested for the indicated phenotypes by FACS analysis of relevant markers and by analysis of secretion of characteristic cytokines.

SEB activation of PBMCs for the generation of soluble CD28—0.1×10\hat{ }6 PBMCs were stimulated with 10-50 ng/mL SEB (Sigma, Cat. No. S4881) for 7 days at 37° C. with/without the indicated concentrations of various treatments and protease inhibitor in 96 well plate. Assays were conducted in complete RPMI-1640 media supplemented with FCS and pen/strep mixture. Protease inhibitor used is TMI-1 (Sigma, Cat. No. PZ0336). When indicated inhibition of CD28 shedding is calculated using the following equation: 100-(sCD283 μM/sCD28basal*100).

Allogeneic Mixed Lymphocyte Reaction—0.1×10\hat{ }6 T cells were mixed with 0.2×10∝mature dendritic cells from different donor for 24-96 hours at 37° C. with/without the indicated concentration of treatments. Assays were conducted in complete RPMI-1640 media supplemented with 10% FCS and pen/strep mixture. When stated, VHH #3C04 (raised against human HER2) was used as an “irrelevant control” and VHH #12B09 was used as a “positive control” for a nanobody with CD28 antagonist effect.

T cells stimulation with HEK/CD80/scOKT3 artificial antigen presenting cells (aAPC-CD80)—1×10\hat{ }5 isolated CD3 T cells (from healthy donors) were stimulated with 0.5×10\hat{ }4 mitomycin treated aAPC-CD80 (HEK293 cells stably transfected with CD80 and scOKT3-CD14 chimera plasmids) for 24-72 hr at 37° C. Treatments of VHH targeting CD28 or controls were added at the indicated concentration in soluble form. When stated, VHH #3C04 (raised against human HER2) was used as an “irrelevant control” and VHH #12B09 was used as a “positive control” for a nanobody with CD28 antagonist effect. Assays were conducted in complete RPMI-1640 media supplemented with 10% FCS and pen/strep mixture.

CD86 blocking FACS—0.25×10\hat{ }6 HEK293 cells stably transfected with human CD28 were incubated with 2 μg/ml biotinylated CD86-Fc (R&D systems, Cat. No. 141-B2) without or with anti-CD28 shedding clone #2A1 in various formats at a fixed concentration of 3 μM for 30 min in room temperature. Cells were washed and taken for secondary binding using streptavidin conjugated to fluorophore (Jackson immuno research, Cat. No. 016-130-084) at 1:500 dilution for 20 min on ice. Incubations were done in a volume of 100 μL in 96-well U bottom plates. Cells were washed twice with 200 μL of FACS buffer and transferred to FACS tubes in 150 μL of FACS buffer for analysis. Cells were analyzed on a Gallios Flow Cytometer (Beckman Coulter) using the Kaluza for Gallios Flow Cytometry Acquisition Software.

Flow Cytometry on CD3 cells—2.5×10⁵ CD3 cells from healthy donors were incubated for 30 min at 37° C. with/without the indicated constructs at mentioned concentrations. Incubations were done in a volume of 100 μL in 96-well U bottom plates. Cells were washed twice with 200 μL of FACS buffer (PBS with 0.05% BSA). The binding of studied constructs to the cells was evaluated by incubation (30 min, on ice) with Nano-Secondary alpaca anti-human IgG recombinant VHH Alexa Flour 647 antibody (Chromotek, Cat. No. srbAF647-1). Incubations were done in dark, at a volume of 100 μL applying concentration recommended by the manufacturer. Following incubation, cells were washed three times with 200 μL of FACS buffer and analyzed on a CytoFLEX Flow Cytometer (Beckman Coulter) using the CytExpert Acquisition and Analysis Software.

MC-38 syngeneic tumor model—MC38 tumor cells were cultured in DMEM and implanted subcutaneously (0.5×10\hat{ }6 cells) into humanized CD28 transgenic C57/B16 mice (genOway)—5 groups of n=10. Six days after inoculation, mice from each group were treated twice a week (200 μg/mice) with/without the shedding blocking agent and anti-PD1 (RMP1-14), for a total of 6 injections. To set a prevention model, one group of mice was also injected with the shedding blocking agent prior to MC-38 inoculation (200 μg/mice, twice). As isotype controls, InVivoMAb human IgG4 (BE0349-BioXcell) or Rat IgG2a (2A3) were used. Tumor volumes were measured three times a week by a calibrated caliper.

VHH affinity maturation—To affinity mature the parental monoclonal anti-CD28 VHH #2A01 llama antibody, the complementarity determining regions (CDRs) residues were randomized by rational mutagenesis using trimer/primer mixes. Libraries design was based on the represented amino acid human and llama natural diversity (Kim et al., “Generation, diversity determination, and application to antibody selection of a human naïve Fab library”, Mol. Cells, 2017, 40(9):655-666; Zemlin et al., “Expressed murine and human CDR-H3 intervals of equal length exhibit distinct repertoires that differ in their amino acid composition and predicted range of structures”, J. Mol. Biol., 2003, 334:733-749; and Tiller et al., “A fully synthetic human Fab antibody library based on fixed VH/VL framework pairings with favorable biophysical properties”, mAbs 20135:445-470, herein incorporated by reference in their entireties). PCR gene assembly protocol was performed using a large number of overlapping oligonucleotides to introduce CDRs diversity. PCR products were cloned into pDCL1 phagemid to create four different phage libraries with mutations concerning CDR1, CDR2, CDR1+CDR2 and CDR3 each with final size of 8.0E+7-1.0E+09. Libraries were QCed by sequencing. Screening for affinity mature variants, i-solutions selections were performed against biotinylated recombinant human CD28-Fc or human CD28 stalk region dimeric peptide captured on Dynabeads™ MyOne™ Streptavidin T 1 Magnetic Beads. Four cycles of selections were performed with decreasing antigen concentrations and increasing free antigen in solution to achieve variants with improved off-rate kinetics. Counter-selection with human IgG were done to reduce background. Enriched CDR1-3 variants were verified by sequencing, biacore, antigen binding in ELISA and efficiency in CD28 shedding blocking activity. Beneficial residues mutations at specific locations in CDR1-3 were taken and randomized with each other to create a combinatorial library lead panel. The combinatorial library was subjected to four cycles of in-solution selections against the human antigen with increasing stringency. 20 variants were isolated and characterized in-depth for and CD28 immune-modulation and CD28-shedding blocking activity. Six top performing clones were further characterized for affinity determination and produced as Fc chimera.

VHH humanization—Protein model of the VHH clone are analyzed to identify residues critical for antibody conformation and binding. Using this information together with in-silico tools for assessing MHC Class II binding, and databases containing antibody segments previously screened using ex vivo T cell immunogenicity assays, a series of humanized heavy chain VHH region sequences are then designed from segments of human V region sequences with the objective that T cell epitopes are avoided.

Biacore—Affinity clones derived from VHH #2A01 affinity maturation libraries was determined using CD28-Fc immobilized on a CM5 Series S sensor chip. Multi-cycle kinetics measurements were done using 5 step dilutions of the analytes (VHH clones) from 200 nM down to 12.5 nM. Kinetic parameters and affinity values were calculated using the 1:1 binding model in Biacore T 200 E valuation software 3.1 by introducing a double (blank channel and blank sample) subtraction.

### Example 1: Affinity Maturation of Single-Domain Antibodies

Three single domain VHH antibodies were previously generated against the stalk domain of the CD28 extracellular domain and were found to be effective in blocking cleavage of membranal CD28 from the surface of cells (see PCT/IL2020/050297, herein incorporated by reference in its entirety). All three VHHs were found to neither substantially agonize nor substantially antagonize CD28 signaling. VHH 2A1 was the most highly studied and the most effective molecule and was therefore selected for affinity maturation of the complementarity determining regions (CDRs).

The full sequence of 2A1 is EVQLVESGGGLVQAGESLRLSCAASGSIASINAMGWYRQAPGSQRELVAAISGGG DTYYADSVKGRFTISRDNAKTTVYLQMNSLRPEDTAVYYCVVDLYGSDYWDWG QGTQVTVSS (SEQ ID NO: 40). This includes a CDR1 of INAMG (SEQ ID NO: 4), a CDR2 of AISGGGDTYYADSVKG (SEQ ID NO: 5) and a CDR3 of DLYGSDYWD (SEQ ID NO: 3).

In order to affinity mature the parental VHH #2A1 llama antibody, the CDRs residues were randomized by rational mutagenesis using trimer/primer mixes. Library design was based on represented amino acid human and llama natural diversity. PCR gene assembly protocol was performed using a large number of overlapping oligonucleotides to introduce CDR diversity. PCR products were cloned into pDCL1 phagemid to create four different phage libraries with mutations in CDR1, CDR2, CDR1+CDR2 and CDR3 each with a final size of 8.0E+7-1.0E+09. Libraries were quality controlled by sequencing. Screening for affinity mature variants in-solutions selections were performed against biotinylated recombinant human CD28-Fc or human CD28 stalk region dimeric peptide captured on Dynabeads™ MyOne™ Streptavidin T1 Magnetic Beads. Four cycles of selections were performed with decreasing antigen concentrations on the beads and increasing free antigen in solution to achieve variants with improved off-rate kinetics. Counter-selection with human IgG was done to reduce background. Enriched CDR1-3 variants were verified by sequencing, Biacore, antigen binding in ELISA and efficiency in CD28 shedding blocking activity. Beneficial residue mutations at specific locations in CDR1-3 were taken and randomized with each other to create a combinatorial library lead panel. The combinatorial library was subjected to four cycles of in-solution selections against the human antigen with increasing stringency. The sequences of the produced variants are provided in Table 1, with the CDRs of each variant provided in Table 2, and affinity constants of seven of the 9 VHH clones summarized in Table 3.

TABLE 1
Full sequences of sdAbs

| VHH Clone | Full Sequence | SEQ ID NO: |
|---|---|---|
| 5A3 | EVQLVESGGGLVQAGESLRLSCAASGSIASINSMGWYRQAP | 26 |
| | GSQRELVAAINEKLLIYYADSVKGRFTISRDNAKTTVYLQMN | |
| | SLRPEDTAVYYCVVDLYGSDYWDWGQGTQVTVSS | |
| 6B3 | EVQLVESGGGLVQAGESLRLSCAASGSIASINAMGWYRQAP | 27 |
| | GSQRELVAAISGGGDTYYADSVKGRFTISRDNAKTTVYLQM | |
| | NSLRPEDTAVYYCVVDMIEQQWWYWGQGTQVTVSS | |
| 6B10 | EVQLVESGGGLVQAGESLRLSCAASGSIASINAMGWYRQAP | 28 |
| | GSQRELVAAISGGGDTYYADSVKGRFTISRDNAKTTVYLQM | |
| | NSLRPEDTAVYYCVVDTHRGVYWYWGQGTQVTVSS | |
| 10E1 | EVQLVESGGGLVQAGESLRLSCAASGSIASIKTMAWYRQAP | 29 |
| | GSQRELVAAINYIKEIYYADSVKGRFTISRDNAKTTVYLQMN | |
| | SLRPEDTAVYYCVVDVTKEDYWYWGQGTQVTVSS | |
| 11E11 | EVQLVESGGGLVQAGESLRLSCAASGSIASINSMAWYRQAP | 30 |
| | GSQRELVAAISNAREVYYADSVKGRFTISRDNAKTTVYLQM | |
| | NSLRPEDTAVYYCVVDVYFQEYWYWGQGTQVTVSS | |
| 11G11 | EVQLVESGGGLVQAGESLRLSCAASGSIASINTMAWYRQAP | 31 |
| | GSQRELVAAINSISRTYYADSVKGRFTISRDNAKTTVYLQMN | |
| | SLRPEDTAVYYCVVDVTKEDYWYWGQGTQVTVSS | |
| 12A9 | EVQLVESGGGLVQAGESLRLSCAASGSIASIKTMAWYRQAP | 32 |
| | GSQRELVTAIASDNRKYYADSVKGRFTISRDNAKTTVYLQM | |
| | NSLRPEDTAVYYCVVDVTKEDYWYWGQGTQVTVSS | |
| 9B3 | EVQLVESGGGLVQAGESLRLSCAASGSIASIRTMAWYRQAP | 33 |
| | GSQRELVAAISSGREVYYADSVKGRFTISRDNAKTTVYLQM | |
| | NSLRPEDTAVYYCVVDMYWQDYWWWGQGTQVTVSS | |
| 9A7 | EVQLVESGGGLVQAGESLRLSCAASGSIASINSMGWYRQAP | 34 |
| | GSQRELVAAISDRSEKYYADSVKGRFTISRDNAKTTVYLQM | |
| | NSLRPEDTAVYYCVVDHHHSDWWTWGQGTQVTVSS | |

TABLE 2
CDRs of sdAbs

| VHH Clone | CDR1 (SEQ ID) | CDR2 (SEQ ID) | CDR3 (SEQ ID) |
|---|---|---|---|
| 5A3 | INSMG (1) | AINEKLLIYYADSVKG (2) | DLYGSDYWD (3) |
| 6B3 | INAMG (4) | AISGGGDTYYADSVKG (5) | DMIEQQWWY (6) |
| 6B10 | INAMG (4) | AISGGGDTYYADSVKG (5) | DTHRGVYWY (7) |
| 10E1 | IKTMA (8) | AINYIKEIYYADSVKG (9) | DVTKEDYWY (10) |
| 11E11 | INSMA (11) | AISNAREVYYADSVKG (12) | DVYFQEYWY (13) |
| 11G11 | INTMA (14) | AINSISRTYYADSVKG (15) | DVTKEDYWY (10) |
| 12A9 | IKTMA (8) | AIASDNRKYYADSVKG (16) | DVTKEDYWY (10) |
| 9B3 | IRTMA (17) | AISSGREVYYADSVKG (18) | DMYWQDYWW (19) |
| 9A7 | INSMG (1) | AISDRSEKYYADSVKG (20) | DHHHSDWWT (21) |

TABLE 3
Affinity constants to CD28-Fc as determined by multi-cycle kinetics

| VHH | ka (1/Ms) | kd (1/s) | KD (M) | KD (nM) | Chi² (RU²) |
|---|---|---|---|---|---|
| 5A03 | 3.38E+04 | 3.78E−05 | 1.117E−09 | 1.12E+00 | 0.03 |
| 9A07 | 5.15E+04 | 9.78E−06 | 1.898E−10 | 1.90E−01 | 0.88 |
| 10E01 | 4.44E+04 | 2.84E−06 | 6.388E−11 | 6.39E−02 | 0.34 |
| 12A09 | 8.34E+04 | 3.43E−07 | 4.104E−12 | 4.10E−03 | 2.70 |
| 11E11 | 4.66E+04 | 1.64E−05 | 3.521E−10 | 3.52E−01 | 0.41 |
| 11G11 | 4.78E+04 | 5.77E−06 | 1.205E−10 | 1.21E−01 | 0.27 |
| 9B03 | 1.12E+05 | 7.17E−08 | 6.421E−13 | 6.42E−04 | 5.37 |

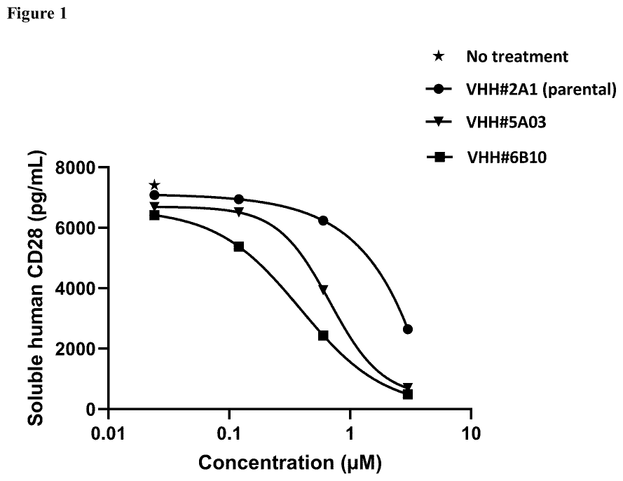

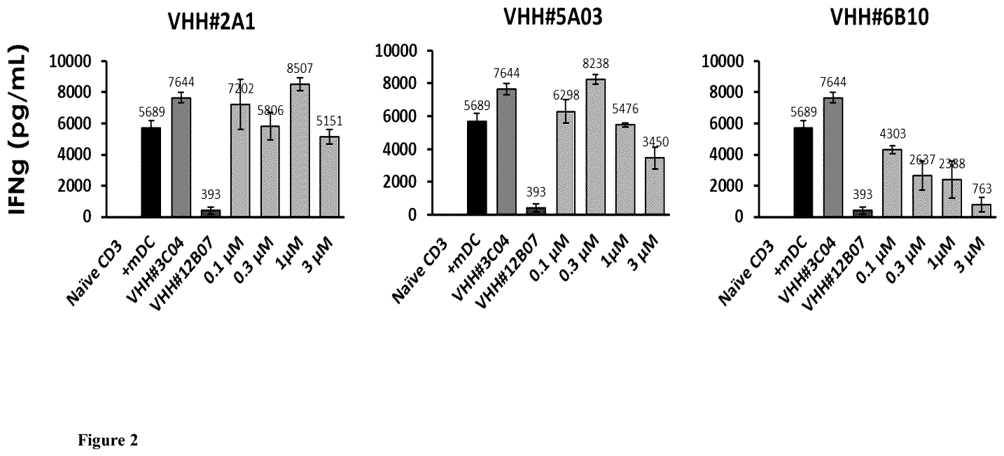

3 affinity matured variants were selected from the first round of mutagenesis, one each with a CDR1+CDR2 mutation (5A3), and two with CDR3 mutations (6B3 and 6B10). From the combinatorial library 20 variants were isolated and characterized in-depth for CD28 immune-modulation and CD28-shedding blocking activity. The 6 best candidates based on CD28 binding and blocking CD28 shedding were selected. All six were found to be superior to the parental VHH with respect to blocking (FIG. 1, 2 representative variants, 5A3 and 6B10, are shown). However, when downstream signaling was tested in a mixed lymphocyte reaction (MLR), the affinity matured VHHs produced a robust antagonistic effect, reducing proinflammatory cytokine secretion, especially at high concentrations (FIG. 2, 2 representative variants, 5A3 and 6B10, are shown).

Blocking cleavage reduces levels of the immunoinhibitory soluble CD28 (sCD28) and thus increases immune cell activation. However, if the cleavage blocking agent also inhibits binding of CD28 to an activating ligand (e.g., CD86, CD80), this antagonistic effect would produce the opposite result and inhibit immune cell activation. These molecules that produced an antagonistic effect therefore may not be useful for immune stimulation, but may be employed in conditions of immune overactivation, such as in autoimmune disease.

### Example 2: Dimeric Binding Agents Reduce CD28 Antagonistic Effect

It was surprisingly discovered that the inclusion of monomer sdAbs within a dimeric binding agent can not only enhance their binding efficiency, but also reduce antagonistic effects. As such, the affinity matured variants were included in Fc chimera molecules and assayed for effects on CD28 signaling.

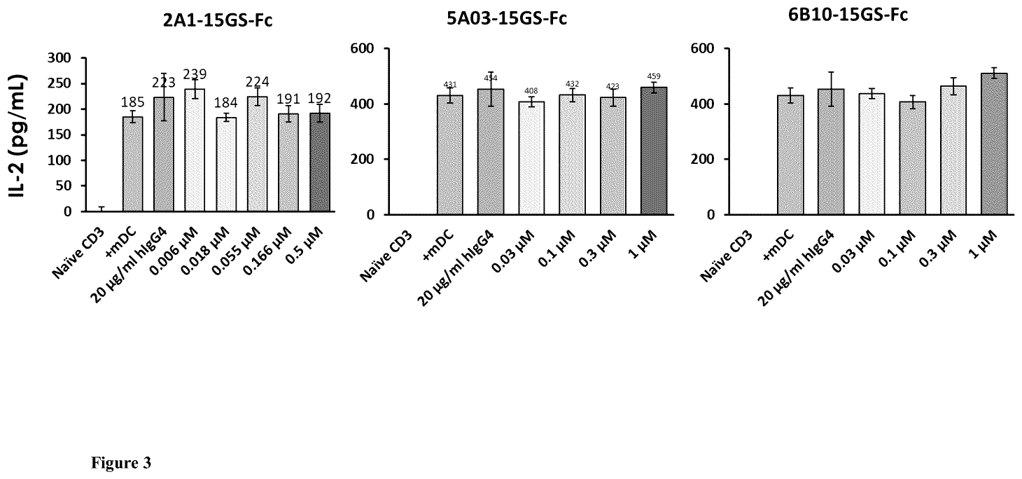

The Fc chimera molecules were generated by cloning the sdAbs upstream to the Fc chain of human IgG1 (SEQ ID NO: 39, Fc with reduced effector function). A flexible amino acid linker (15 amino acid GGGGS linker; for simplicity GGGGS repeat linkers are hereinafter referred to as GS linkers) was inserted between the sdAb C-terminus and the N-terminus of the Fc. These dimeric agents were tested in the MLR experiment. Surprisingly, the inclusion of the Fc domain abolished the antagonistic effect that had been seen with the monomeric sdAbs (FIG. 3, 2 representative variants, 5A3 and 6B10, are shown). As such, these dimeric agents are highly effective immunostimulants as they reduce sCD28 production and do not interfere with membranal CD28 (mCD28) activation.

### Example 3: Affinity Matured Clones 12A09-Fc and 9B03-Fc Inhibit mCD28 Cleavage

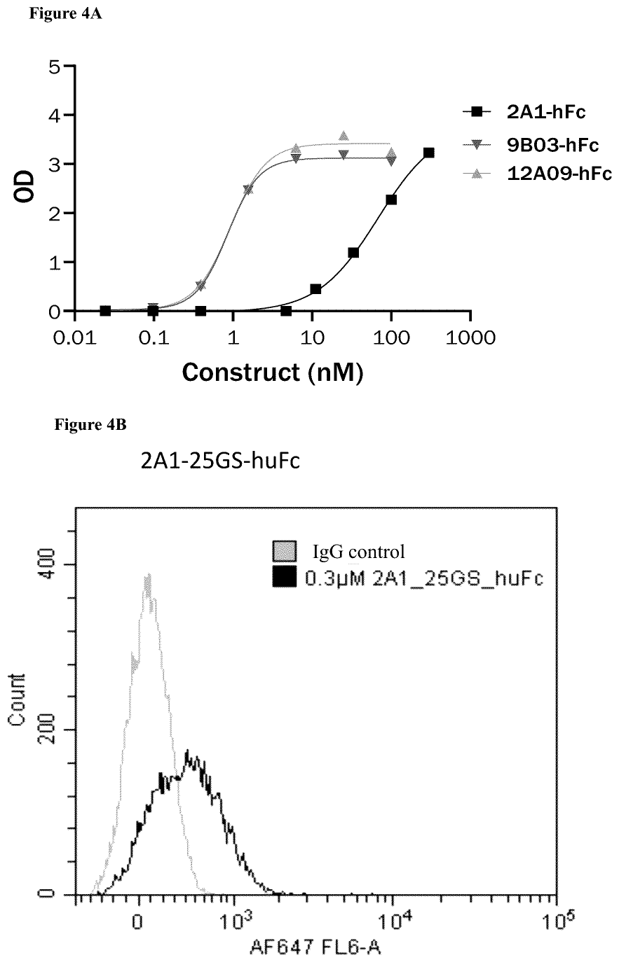

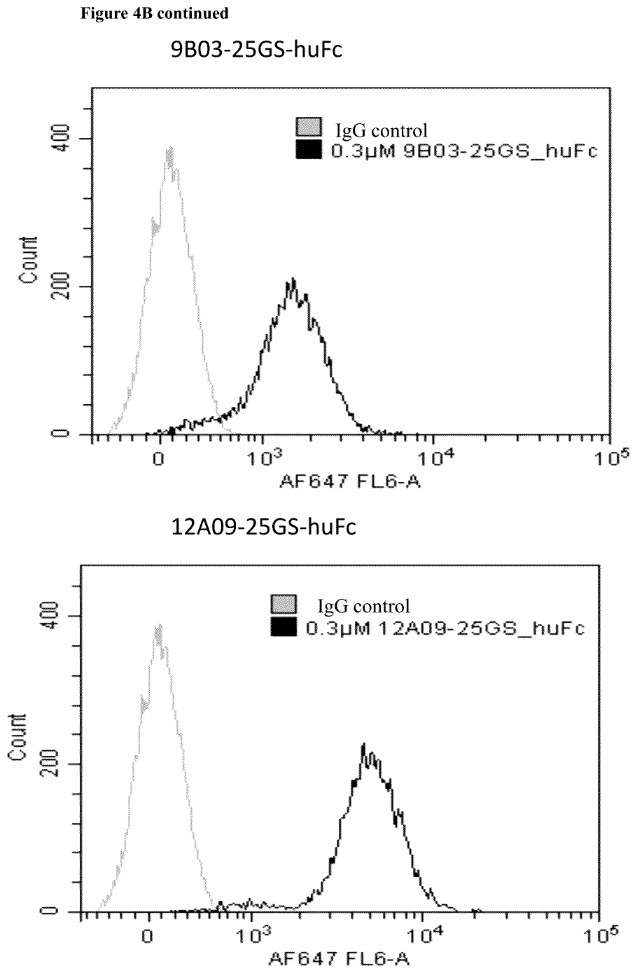

Next, the ability of the affinity matured clones fused to Fc to bind CD28 was tested. The 12A9 and 9B3 clones were cloned upstream of the Fc chain of human IgG4. IgG4 was selected as it is a generally non-cytotoxic Fc. Two-point mutations (S228P and L235E) were generated in the IgG4 chain in order to further reduce cytotoxicity and enhance stability/reduce aggregation. A 25 amino acid linker (GGGGSx5) was inserted between the VHH and the Fc. The binding of these two molecules to CD28 was compared to the binding of the parental VHH construct. As expected, the constructs comprising the two affinity matured VHHs bound CD28 significantly better in solution (FIG. 4A) and on the cell surface (FIG. 4B).

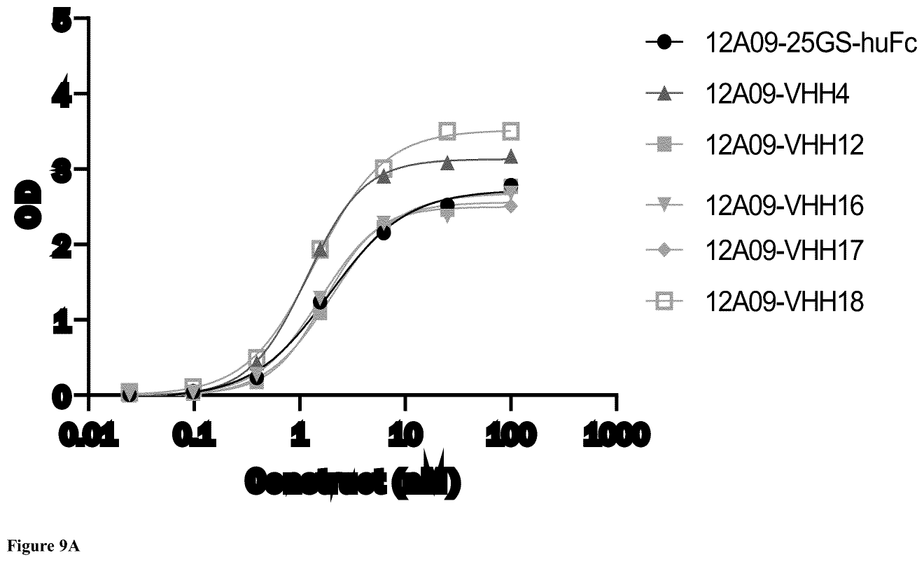

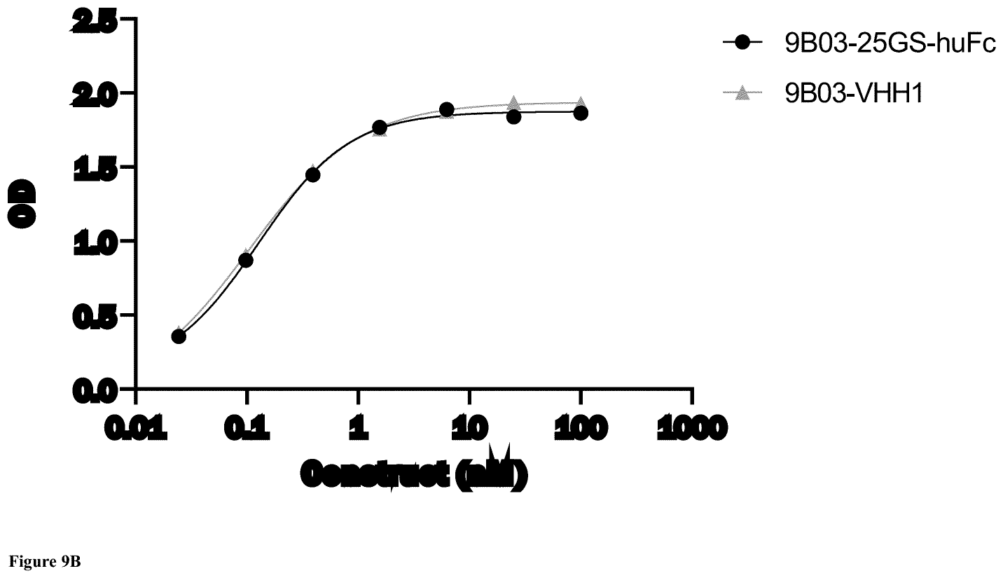

The ability of the dimeric fusion proteins to actually bind mCD28 on the cell surface and block cleavage was tested. Staphylococcal enterotoxin B (SEB) activates peripheral blood mononuclear cell (PBMCs) and induces CD28 cleavage and sCD28 shedding. The addition of the dimeric molecule comprising the parental VHH (2A1, 100 nm concentration) decreases sCD28 production indicating genuine surface binding and cleavage blocking (FIG. 5A-5B). Both 12A9-25GS-hIgG4 (FIG. 5A) and 9B3-25GS-hIgG4 (FIG. 5B) were superior to the parental molecule and produced a robust inhibition of CD28 cleavage in a dose dependent-manner.

### Example 4: Dimeric Binding Agents are not Agonistic

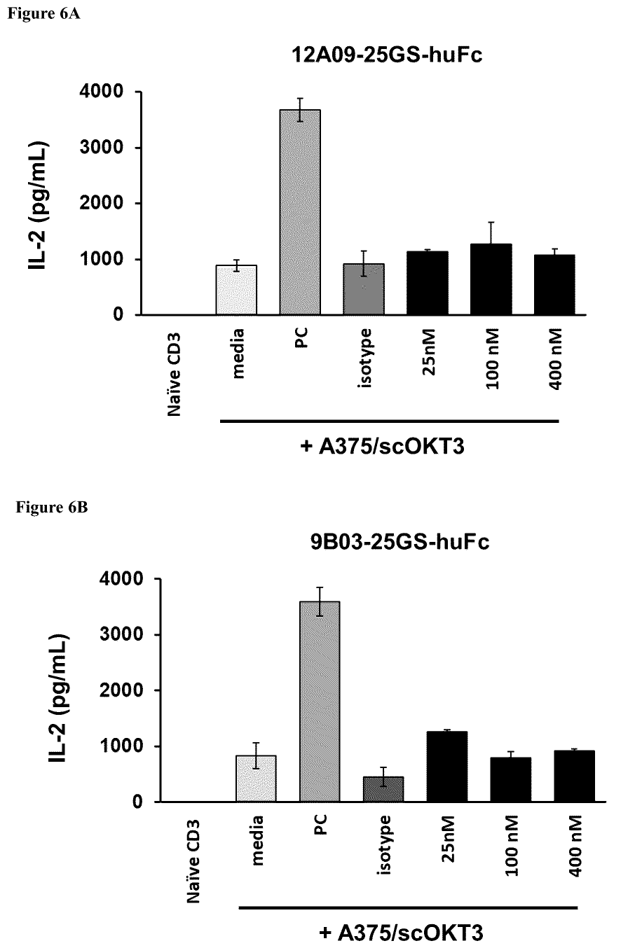

Having established that the dimeric binding agents bind CD28 at a higher affinity than the monomeric VHHs, produce improved CD28 cleavage blocking and do not produce an antagonistic effect on CD28, the potential of these agents to act agonistically was tested. Isolated human CD3 cells were stimulated for 2 days with cells of the A375 cell line over expressing scOKT3 (anti-CD3). This stimulation was performed in the presence of an anti-CD28 agonist antibody, clone 28.2, which served as a positive control, an irrelevant human IgG5 which served as a negative control or Fc chimera affinity mature clone VHHs (25 amino acid linker). The concentration of human IL-2 secreted into the supernatant was quantified with standardized sandwich ELISA (Biolegend). As expected, clone 28.2 produced robust IL-2 secretion, however, the Fc chimera did not show a significant agonistic effect (FIG. 6A-6B, clones 12A9 and 9B3 are shown).

### Example 5: Linker Length Examination

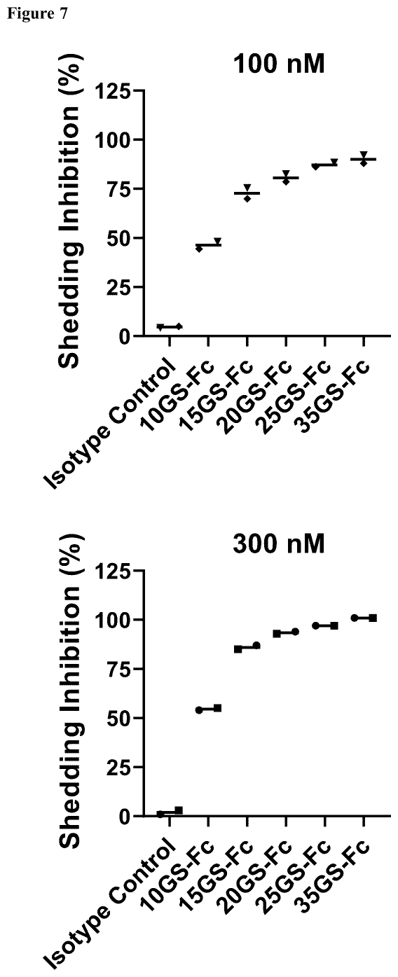

As both a 15 amino acid GGGGS linker and a 25 amino acid GGGGS linker (referred to herein throughout as GS linkers) both produced effective cleavage blocking, the optimal size of the linker was investigated. Fc chimera variants of VHH 12A9 were produced with different lengths of flexible linkers between the Fc and VHH modules. Linkers from 10 amino acids to 35 were investigated. SEB activated PBMCs were again used to measure CD28 cleavage from the cell surface. The chimeric molecules were introduced to the PBMCs at two different concentrations (100 nM and 300 nM) and sCD28 levels were measured. At both concentrations it was clearly observed that increasing the linker length produced greater levels of shedding inhibition, with linkers of 25 amino acids or longer producing greater than 90% shedding inhibition (FIG. 7). As maximal shedding inhibition was achieved with the 35 amino acids linker, there is no reason to use linkers of even greater length.

### Example 6: In Vivo Examination

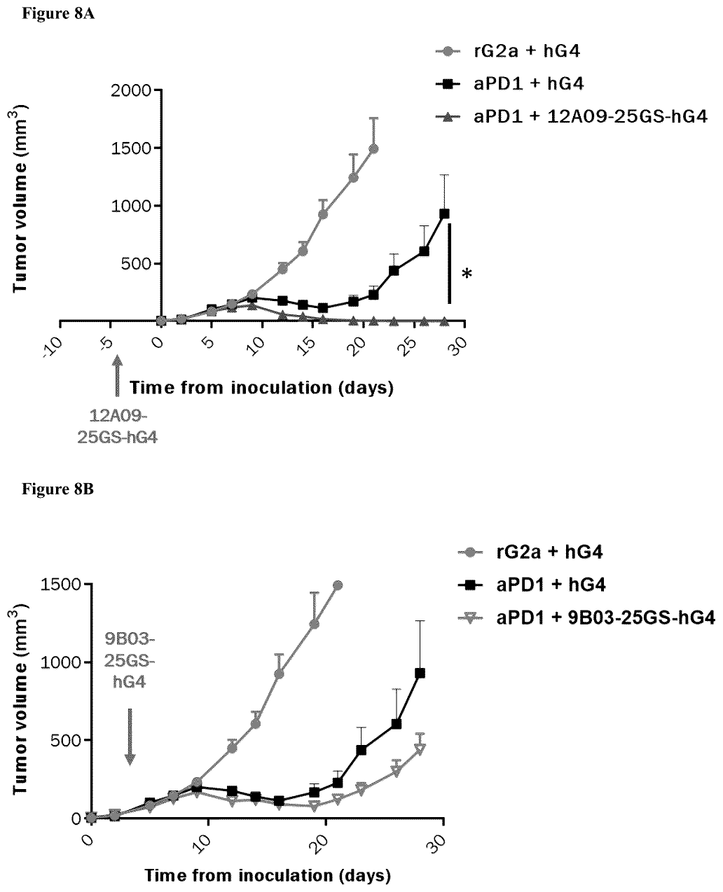

The therapeutic potential of these dimeric VHH constructs was further examined in-vivo in a MC-38 syngeneic model, in which MC38 tumor cells are subcutaneously implanted into humanized CD28 transgenic mice. Anti-PD1 (RMP1-14) treatment, starting 6 days post-implantation, hindered the rate of the tumor growth compared to the corresponding isotype control (rG2a is the rat isotype control for the PD-1 antibody) (FIG. 8A-8B). Surprisingly, a prophylactic treatment with 12A9-25GS-hIgG4 5 days before implantation of the cancer cells, in combination with the anti-PD1 treatment, resulted in complete prevention of cancer growth (FIG. 8A). All cancer cells were killed by the mice's immune system such that even by day 30 no cancer was detected. A post-implantation combined treatment (both at 6 days) significantly hindered the tumor growth and was superior to treatment with anti-PD1 alone (FIG. 8B). These in vivo results further support that the dimeric agents do not have an antagonistic effect on CD28, as such an effect would result in tumor growth and not shrinkage/prevention. As such, it is fully clear that these agents block CD28 cleavage from the cell surface and do so without antagonizing CD28 function which leads to robust cancer treatment.

### Example 7: VHH Humanization

Two of the VHH clones, 12A9 and 93 were further humanized such that the camelid backbone of the VHH was removed. To this end, protein models of the heavy chain VHH region were analyzed to identify residues critical for antibody conformation and binding. Using this information together with in silico technologies (tool 1: assessing MHC Class II binding. Tool2: database containing antibody segments previously screened using ex vivo T cell immunogenicity assays), a series of humanized heavy chain VHH region sequences were designed from segments of human V region sequences with the objective that T cell epitopes will be avoided. Five clones were received for 12A9 and 4 clones for 91B3. The sequences of these clones are provided in Table 4.

TABLE 4
Full sequences of humanized clones

| | | SEQ |
|---|---|---|
| VHH clone | Full sequence | ID NO: |
| VHH | EVQLVESGGGLVQPGGSLRLSCAASGSIASIKTMA | 70 |
| 12A09_VHH4 | WYRQAPGKQRELVTAIASDNRKYYADSVKGRFTI | |
| | SRDNSKNTLYLQMNSLRAEDTAVYYCVVDVTKE | |
| | DYWYWGQGTLVTVSS | |
| VHH | EVQLVESGGGLVQPGGSLRLSCKASGSIASIKTMA | 71 |
| 12A09_ VHH12 | WYRQAPGKGLELVTAIASDNRKYYADSVKGRFTI | |
| | SRDNSKTTVYLQMNSLRAEDTAVYYCVVDVTKE | |
| | DYWYWGQGTLVTVSS | |
| VHH | EVQLVESGGGLVQPGGSLRLSCAASGSTASIKTM | 72 |
| 12A09_ VHH16 | AWYRQAPGKGLELVTAIASDNRKYYADSVKGRF | |
| | TISRDNSKTTVYLQMNSLRAEDTAVYYCVVDVT | |
| | KEDYWYWGQGTLVTVSS | |
| VHH | EVQLVESGGGLVQPGGSLRLSCKASGSTASIKTM | 73 |
| 12A09_ VHH17 | AWYRQAPGKGLELVTAIASDNRKYYADSVKGRF | |
| | TISRDNSKTTVYLQMNSLRAEDTAVYYCVVDVT | |
| | KEDYWYWGQGTLVTVSS | |
| VHH | EVQLVESGGGLVQPGGSLRLSCAASGSIASIKTMA | 74 |
| 12A09_ VHH18 | WYRQAPGKGRELVTAIASDNRKYYADSVKGRFTI | |
| | SRDNSKTTVYLQMNSLRAEDTAVYYCVVDVTKE | |
| | DYWYWGQGTLVTVSS | |
| VHH | EVQLVESGGGLVQPGESLRLSCAASGSIASIRTMA | 75 |
| 9B03_VHH1 | WYRQAPGSQRELVAAISSGREVYYADSVKGRFTI | |
| | SRDNAKTTVYLQMNSLRAEDTAVYYCVVDMYW | |
| | QDYWWWGQGTQVTVSS | |
| VHH 9B03 | EVQLVESGGGLVQPGGSLRLSCKASGSIASIRTMA | 76 |
| VHH12 | WYRQAPGKGLELVAAISSGREVYYADSVKGRFTI | |
| | SRDNSKTTVYLQMNSLRAEDTAVYYCVVDMYW | |
| | QDYWWWGQGTLVTVSS | |
| VHH 9B03 | EVQLVESGGGLVQPGGSLRLSCKASGSTASIRTM | 77 |
| VHH19 | AWYRQAPGKGLELVSAISSGREVYYADSVKGRFT | |
| | ISRDNSKTTVYLQMNSLRAEDTAVYYCVVDMYW | |
| | QDYWWWGQGTLVTVSS | |
| VHH 9B03 | EVQLVESGGGLVQPGGSLRLSCAASGSIASIRTMA | 78 |
| VHH20 | WYRQAPGKGLELVSAISSGREVYYADSVKGRFTI | |
| | SRDNSKTTVYLQMNSLRAEDTAVYYCVVDMYW | |
| | QDYWWWGQGTLVTVSS | |

Dimeric binding agents were also produced with the humanized VHH variants. Five 12A9 humanized variants and one 91B3 humanized variant were linked to the human IgG4 Fe via a 25 amino acid GS flexible linker. Binding of the humanized variant chimeras to human CD28 was confirmed using direct CD28 EIA as before. The 5 tested 12A9 humanized variants and 1 tested 9B33 humanized variant all bound CD28 at least as well as their parental (camelid) variant chimera (FIG. 9A-9B). Indeed, 12A9-VHH18-25GS-huFc and 12A9-VHH4-25GS-huFc were both measurably superior to the parental 12A9 VHH chimera. The affinity constants of the six dimeric agents with humanized variants and the agents with the parental VHHs are summarized in Table 5.

TABLE 5
Affinity constants to CD28-Fc as determined by multi-cycle kinetics

| | | | | Chi² |
|---|---|---|---|---|
| VHH | ka (1/Ms) | kd (1/s) | KD (M) | (RU²) |
| 12A09-25GS-hG4 | 2.30E+05 | 3.71E−05 | 1.61E−10 | 0.69 |
| 12A09 (VHH4)-25GS-hG4 | 1.24E+05 | 5.38E−05 | 4.35E−10 | 0.45 |
| 12A09 (VHH12)-25GS-hG4 | 7.61E+04 | 3.01E−05 | 3.96E−10 | 0.03 |
| 12A09 (VHH16)-25GS-hG4 | 8.49E+04 | 2.53E−05 | 2.98E−10 | 0.33 |
| 12A09 (VHH17)-25GS-hG4 | 7.59E+04 | 1.29E−05 | 1.70E−10 | 0.36 |
| 12A09 (VHH18)-25GS-hG4 | 1.17E+05 | 4.22E−05 | 3.61E−10 | 0.42 |
| 9B03-25GS-hG4 | 2.6E+05 | 1.02E−04 | 3.927E−10 | 0.80 |
| 9B03 (VHH1)-25GS-hG4 | 2.19E+05 | 9.79E−05 | 4.487E−10 | 0.80 |

Binding to CD28 on the surface of cells was also evaluated as before. CD3 cells were analyzed by FACS in presence of isotype control, the humanized VHH chimera or the parental VHH chimera (300 nM). The mean fluorescent intensity measured for these various agents is summarized in Tables 6 and 7. All of the humanized VHH chimera were at least as good binders as the parental VHH chimera, and indeed 12A09-VHH12-25G-huFc, 12A09-VHH16-25G-huFc (humanized), and 12A09-VHH17-25G-huFc produced three times the fluorescence as the parental constructs.

TABLE 6
Binding of 12A9 humanized chimeras
to CD28 on the surface of cells.

| Construct | MFI |
|---|---|
| Isotype hIgG4 | 71.9 |
| 12A09-25GS-huFc (parental) | 517.3 |
| 12A09-VHH4-25GS-huFc (humanized) | 564.1 |
| 12A09-VHH12-25GS-huFc (humanized) | 1652.3 |
| 12A09-VHH16-25GS-huFc (humanized) | 1586.8 |
| 12A09-VHH17-25GS-huFc (humanized) | 1915.6 |
| 12A09-VHH18-25GS-huFc (humanized) | 612.2 |

TABLE 7
Binding of 9B3 humanized chimera
to CD28 on the surface of cells.

| Construct | MFI |
|---|---|
| Isotype hIgG4 | 115.8 |
| 9B03-25GS-huFc | 953 |
| 9B03-VHH1-25GS-huFc (humanized) | 1058 |

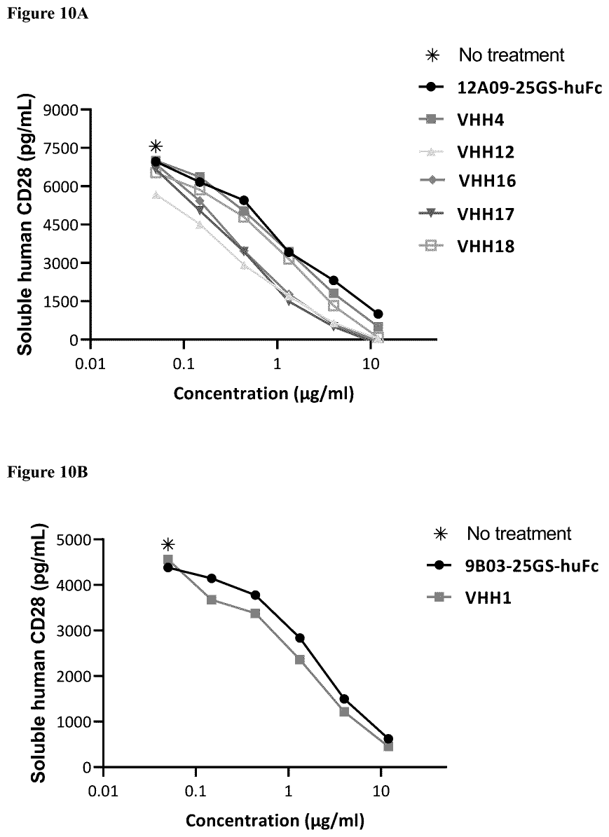

The ability of the humanized variant chimeras to block CD28 shedding was also examined. PMBCs were stimulated with SEB as before in the presence of the humanized variants. The 5 tested 12A9 humanized variants and 1 tested 9B3 humanized variant in Fc chimeras all blocked CD28 shedding from the cell surface at least as well as the parental variant chimera (FIG. 10A-10B). Indeed, the humanized variants produced slightly improved shedding blocking, especially at intermediate concentrations, with 12A9-VHH12, 12A9-VHH16 and 12A9-VHH17 showing the most improvement over the parental VHH (FIG. 10A).

### Example 8: Generation of Dimeric Single-Chain sdAb Molecules

Three single domain VHH antibodies were previously generated against the stalk domain of the CD28 extracellular domain and were found to be effective in blocking cleavage of membranal CD28 from the surface of cells (see PCT/IL2020/050297, herein incorporated by reference in its entirety). All three VHHs were found to neither substantially agonize nor substantially antagonize CD28 signaling. The sequences of these single domain antibodies (sdAb) are provided in Table 8 and the CDRs of these sdAbs are provided in Table 9.

TABLE 8
Full sequences of sdAbs

| VHH Clone | Full Sequence | SEQ ID NO: |
|---|---|---|
| 2A1 | EVQLVESGGGLVQAGESLRLSCAASGSIASINAMGWYRQAP | 40 |
| | GSQRELVAAISGGGDTYYADSVKGRFTISRDNAKTTVYLQM | |
| | NSLRPEDTAVYYCVVDLYGSDYWDWGQGTQVTVSS | |
| 4A4 | EVQLVESGGGLVQAGGSLRLSCAASGSLFSINAMAWYRQAP | 95 |
| | GKQRELVAAITSSGSTNYANSVKGRFTVSRDNAKNTMYLQM | |
| | NSLKPEDTAVYYCVVDEYGSDYWIWGQGTQVTVSS | |
| 4A1 | QVQLVESGGGLVQAGGSLRLSCAASGSIFSINAMGWYRQAP | 96 |
| | GKQRERVAAITSGGSTNYADSVKGRFTISRDNAKNTVYLQM | |
| | NNLEPRDAGVYYCVVDLYGEDYWIWGQGTQVTVSS | |

TABLE 9
CDRs of sdAbs

| VHH Clone | CDR1 (SEQ ID) | CDR2 (SEQ ID) | CDR3 (SEQ ID) |
|---|---|---|---|
| 2A1 | INAMG (4) | AISGGGDTYYADSVKG (5) | DLYGSDYWD (3) |
| 4A4 | INAMA (98) | AITSSGSTNYANSVKG (99) | DEYGSDYWI (100) |
| 4A1 | INAMG (4) | AITSGGSTNYADSVKG (101) | DLYGEDYWI (102) |

In order to improve the specificity and efficacy of the cleavage blocking VHHs, it was decided to generate a dimeric agent comprising two copies of the VHH. As VHH 2A1 was found to be the most effective cleavage blocking agent, it was selected for inclusion in the dimeric molecule.

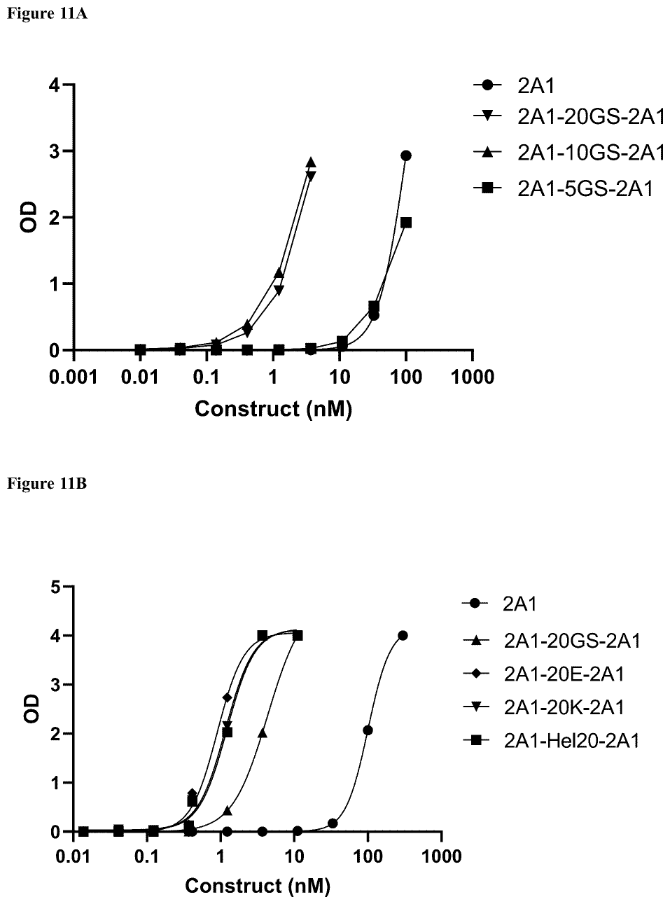

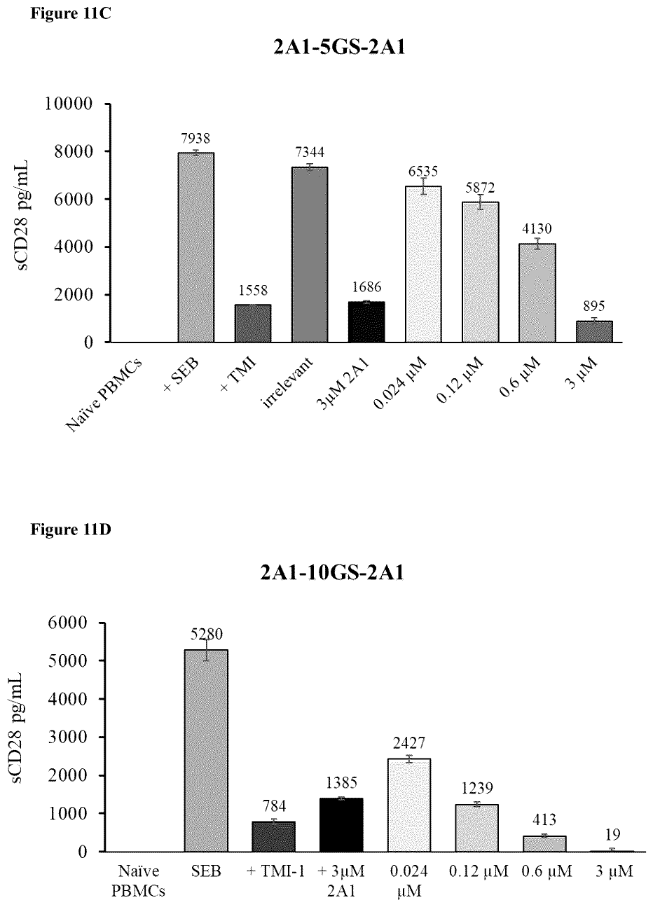

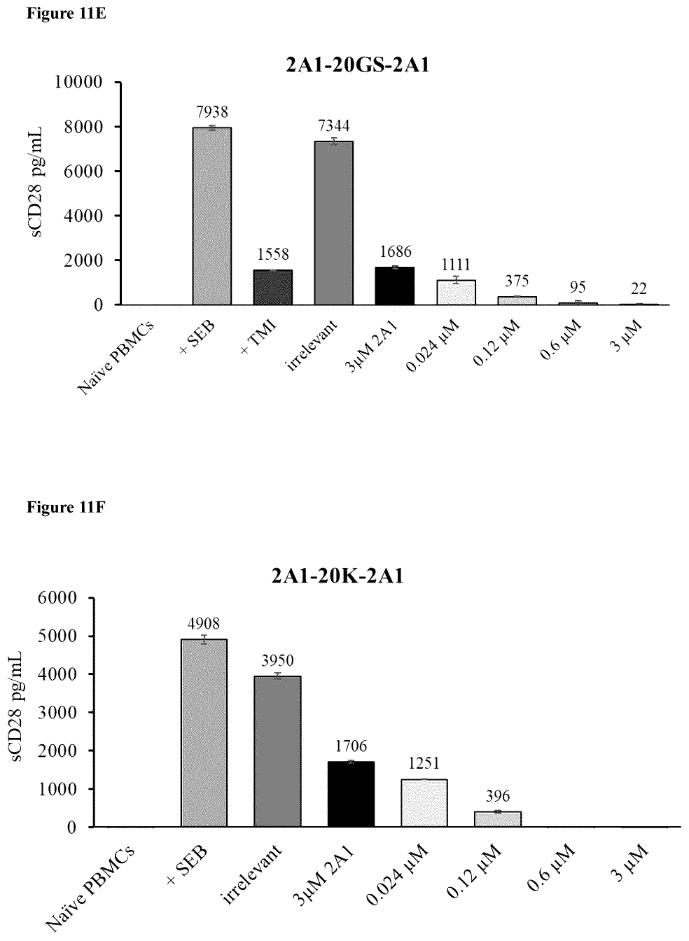

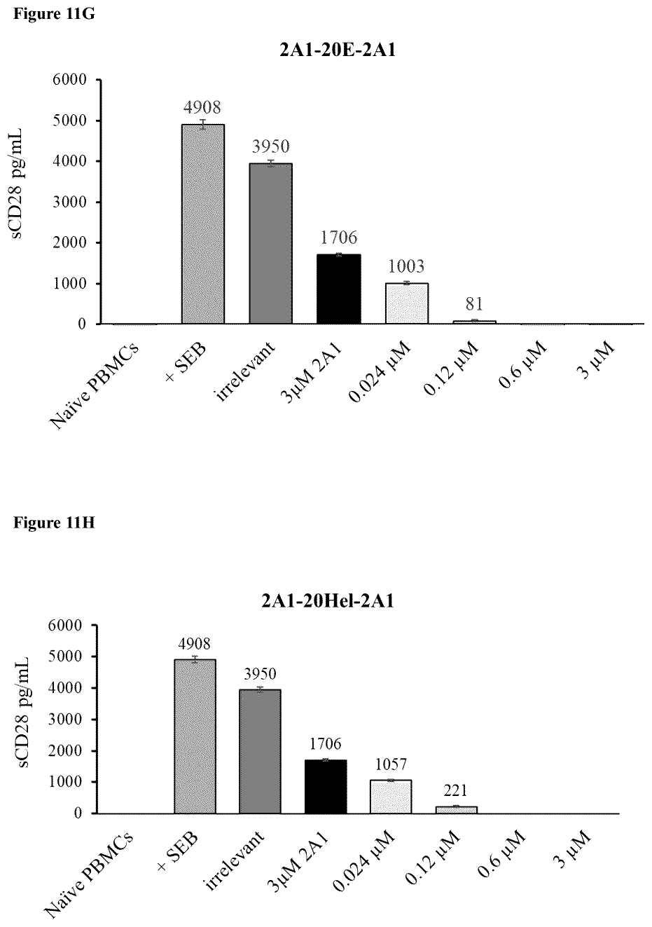

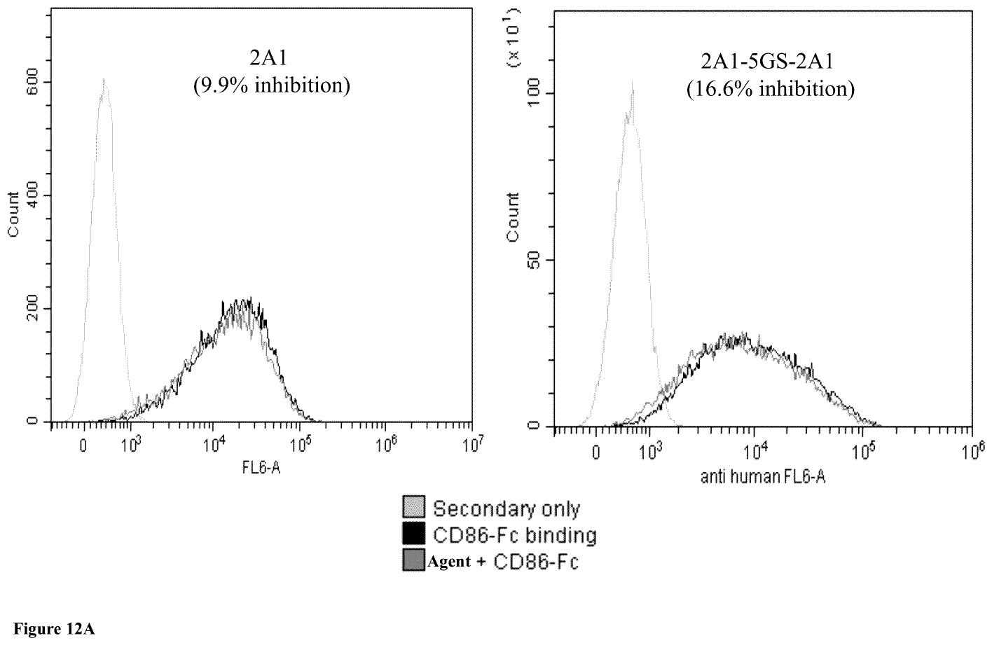

First, single-chain tandem dimeric constructs were generated in which the C-terminus of a first 2A1 is connected via a peptide linker to the N-terminus of a second 2A1. Initially a flexible (G₄S) linker was tested. Dimeric single-chain molecules were generated with 1, 2 or 4 linker repeats. The sequences of the various single chain molecules are summarized in Table 10. These dimeric constructs were tested for binding to a recombinant CD28 fused to an Fc backbone and were compared to the monomeric 2A1 as a control. The very short 5 amino acid linker did not produce increased affinity for CD28 and was comparable to the monomeric 2A1 (FIG. 11A). In contrast, the 10 or 20 amino acids linker produced increased binding as compared to the monomer (FIG. 11A). The difference in EC50 is summarized in Table 11.

TABLE 10
Single-chain dimeric agents

| | | SEQ |
|---|---|---|
| | | ID |
| Name | Full Sequence | NO: |
| 2A1- | EVQLVESGGGLVQAGESLRLSCAASGSIASINAMGWYRQAPGS | 103 |
| 5GS- | QRELVAAISGGGDTYYADSVKGRFTISRDNAKTTVYLQMNSLRP | |
| 2A1 | EDTAVYYCVVDLYGSDYWDWGQGTQVTVSSAAAGGGGSEVQ | |
| | LVESGGGLVQAGESLRLSCAASGSIASINAMGWYRQAPGSQREL | |
| | VAAISGGGDTYYADSVKGRFTISRDNAKTTVYLQMNSLRPEDT | |
| | AVYYCVVDLYGSDYWDWGQGTQVTVSS | |
| 2A1- | EVQLVESGGGLVQAGESLRLSCAASGSIASINAMGWYRQAPGS | 104 |
| 10GS- | QRELVAAISGGGDTYYADSVKGRFTISRDNAKTTVYLQMNSLRP | |
| 2A1 | EDTAVYYCVVDLYGSDYWDWGQGTQVTVSSAAAGGGGSGGG | |
| | GSEVQLVESGGGLVQAGESLRLSCAASGSIASINAMGWYRQAP | |
| | GSQRELVAAISGGGDTYYADSVKGRFTISRDNAKTTVYLQMNS | |
| | LRPEDTAVYYCVVDLYGSDYWDWGQGTQVTVSS | |
| 2A1- | EVQLVESGGGLVQAGESLRLSCAASGSIASINAMGWYRQAPGS | 105 |
| 20GS- | QRELVAAISGGGDTYYADSVKGRFTISRDNAKTTVYLQMNSLRP | |
| 2A1 | EDTAVYYCVVDLYGSDYWDWGQGTQVTVSSAAAGGGGSGGG | |
| | GSGGGGSGGGGSEVQLVESGGGLVQAGESLRLSCAASGSIASIN | |
| | AMGWYRQAPGSQRELVAAISGGGDTYYADSVKGRFTISRDNAK | |
| | TTVYLQMNSLRPEDTAVYYCVVDLYGSDYWDWGQGTQVTVSS | |
| 2A1- | EVQLVESGGGLVQAGESLRLSCAASGSIASINAMGWYRQAPGS | 106 |
| 20K- | QRELVAAISGGGDTYYADSVKGRFTISRDNAKTTVYLQMNSLRP | |
| 2A1 | EDTAVYYCVVDLYGSDYWDWGQGTQVTVSSAAAGGGKSGGG | |
| | KSGGGKSGGGKSEVQLVESGGGLVQAGESLRLSCAASGSIASIN | |
| | AMGWYRQAPGSQRELVAAISGGGDTYYADSVKGRFTISRDNAK | |
| | TTVYLQMNSLRPEDTAVYYCVVDLYGSDYWDWGQGTQVTVSS | |
| 2A1- | EVQLVESGGGLVQAGESLRLSCAASGSIASINAMGWYRQAPGS | 107 |
| 20E- | QRELVAAISGGGDTYYADSVKGRFTISRDNAKTTVYLQMNSLRP | |
| 2A1 | EDTAVYYCVVDLYGSDYWDWGQGTQVTVSSAAAGGGESGGG | |
| | ESGGGESGGGESEVQLVESGGGLVQAGESLRLSCAASGSIASINA | |
| | MGWYRQAPGSQRELVAAISGGGDTYYADSVKGRFTISRDNAKT | |
| | TVYLQMNSLRPEDTAVYYCVVDLYGSDYWDWGQGTQVTVSS | |
| 2A1- | EVQLVESGGGLVQAGESLRLSCAASGSIASINAMGWYRQAPGS | 108 |
| 20Hel- | QRELVAAISGGGDTYYADSVKGRFTISRDNAKTTVYLQMNSLRP | |
| 2A1 | EDTAVYYCVVDLYGSDYWDWGQGTQVTVSSAAAGGGGSAEA | |
| | AAKEAAAKEAAAKAAAGSGGGGSEVQLVESGGGLVQAGESLR | |
| | LSCAASGSIASINAMGWYRQAPGSQRELVAAISGGGDTYYADSV | |
| | KGRFTISRDNAKTTVYLQMNSLRPEDTAVYYCVVDLYGSDYW | |
| | DWGQGTQVTVSS | |

TABLE 11
EC50 values for CD28 protein binding of different
flexible linker constructs. Values given in nM.

| | | 2A1-5GS- | 2A1- | 2A1-20GS- |
|---|---|---|---|---|
| | 2A1 | 2A1 | 10GS-2A1 | 2A1 |
| EC50 | 96.79 | 72.60 | 4.46 | 7.51 |

The effect on cleavage was directly measured in cells. Peripheral blood mononuclear cells (PBMCs) were collected from healthy donor and stimulated with Staphylococcal Enterotoxin B (SEB) to activate the immune cells and produce robust levels of sCD28. Addition of pan-metalloprotease inhibitor TMI, or monomeric 2A1 (3 μM) both greatly reduced sCD28 levels, while an irrelevant VHH had no effect (FIG. 11C-11E). The 20GS construct was as potent as the TMI inhibitor and the monomeric 2A1 even at the lowest concentration tested and at higher concentrations nearly abolished sCD28 production showing it to be a more potent cleavage inhibitor than the monomer (FIG. 11E). The 10GS construct was not quite as effective at the lowest concentration, but at higher concentrations also essentially abolished CD28 cleavage and was still considerably superior to the monomer (FIG. 11D). The 5GS construct, as expected based on the binding data, was the least effective, but at high concentrations was slightly superior to the TMI inhibitor and the monomer (FIG. 11C). These results indicate that the single-chain dimeric molecules are effective in blocking sCD28 production, even more so than the monomeric form of the VHH.

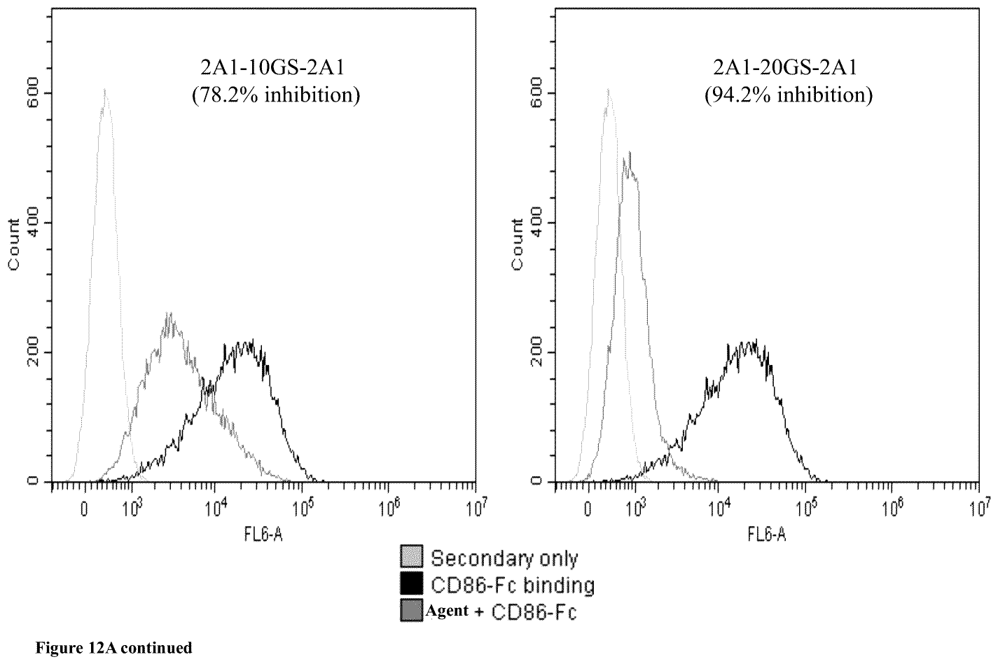

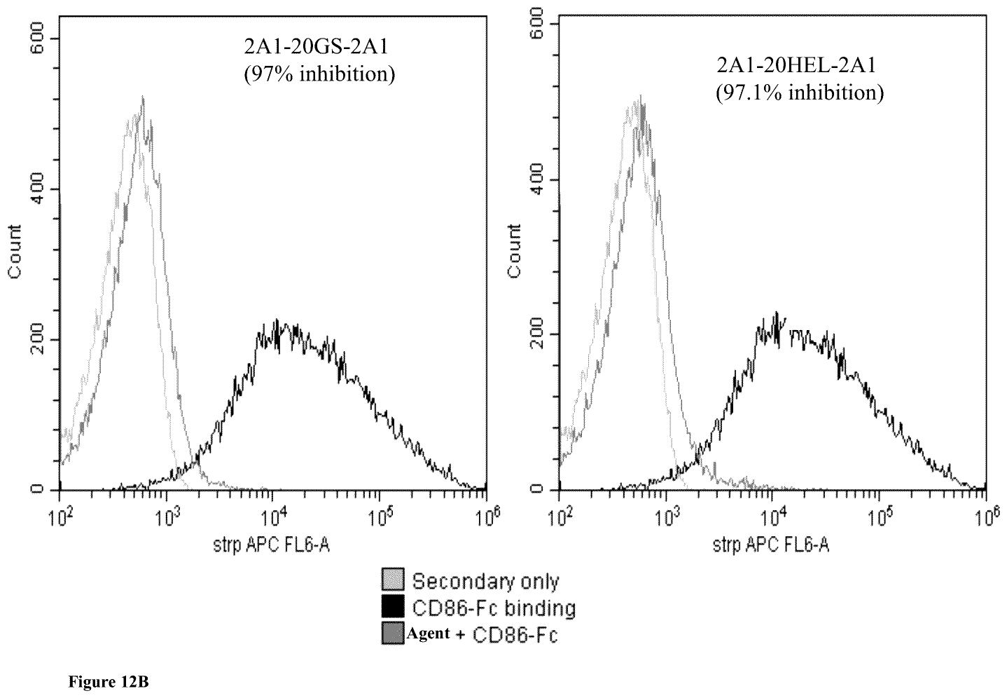

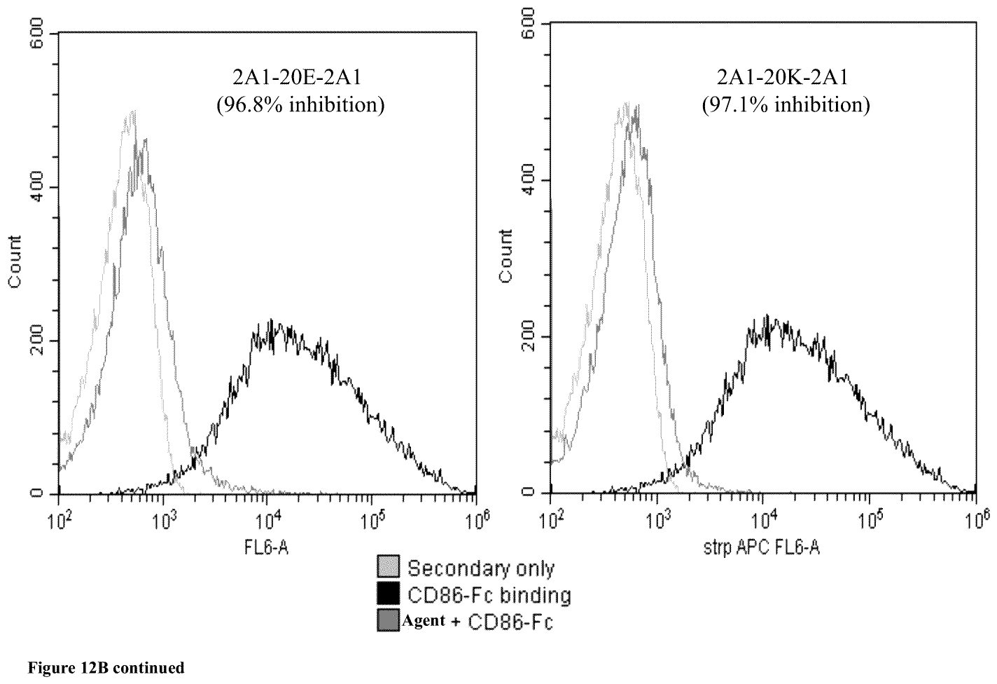

Next, these single-chain dimeric agents were tested to see if they produced an antagonistic effect on CD28. Blocking cleavage would reduce levels of the immunoinhibitory soluble CD28 (sCD28) and thus increase immune cell activation. However, if the cleavage blocking agent also inhibits binding of CD28 to an activating ligand (e.g., CD86), this antagonistic effect would produce the opposite result and inhibit immune cell activation. VHH 2A1 is known not to produce an antagonistic effect and did not block binding of recombinant CD86 to HEK cells overexpressing human CD28 (FIG. 12A). Unexpectedly, the single-chain dimeric molecules with 10 or 20 amino acids linkers produced a robust inhibition of CD86 binding, with over 75% inhibition of binding (FIG. 12A). The short linker did not substantially inhibit CD86 binding and looked largely similar to monomeric 2A1 (FIG. 12A).

It was hypothesized that the antagonistic effect was caused by an interaction between the linker and ligand binding regions of CD28. In order to avoid this interaction, two additional single-chain dimeric agents were created. In these molecules, the 20 amino acid long linker with 4 G₄S repeats has every 4^{th} G replaced with a charged amino acid. In one construct the linker was made positive with glycine amino acids replaced by lysine amino acids (20K) and in the other, the linker was made negative with glycine amino acids replaced by glutamic acid amino acids (20E). It was hypothesized that the charged amino acids would produce electrostatic repulsion from the CD28 extracellular domain and thus abrogate any antagonist effect. A third new single-chain dimeric agent was also created, but a rigid helical linker was used in place of a flexible linker (Hel20). Sequences of these dimeric agents are provided in Table 10.

The same binding assay was repeated with these molecules and the 20GS agent and monomeric 2A1 molecule were used as controls (FIG. 11B). Regardless of the charge or rigidity of the linker used, all the long linker constructs behaved similarly, producing superior target binding as compared to the monomeric 2A1. Indeed, the two charged linkers and the rigid linker constructs actually were slightly superior to the 20GS molecule (FIG. 11B). The EC50 values of these constructs are summarized in Table 12. The new agents all also inhibited sCD28 production as expected (FIG. 11F-11H).

TABLE 12
EC50 values for CD28 protein binding of different
charged/rigid linker constructs. Values given in nM.

| | | 2A1-20E- | 2A1-20K- | 2A1-Hel20- | 2A1-20GS- |
|---|---|---|---|---|---|
| | 2A1 | 2A1 | 2A1 | 2A1 | 2A1 |
| EC50 | 100.6 | 0.902 | 1.165 | 1.22 | 4.346 |

As all the new linker molecules still demonstrated enhanced cleavage blocking, they were tested for an antagonist effect on CD28-CD86 binding as before. Contrary to what was expected, all three new linker molecules produced robust CD86 blocking, with levels of inhibition comparable to the flexible 20 amino acid linker (FIG. 12B). These results indicate that regardless of the structure of the linker, a longer linker used in a single-chain VHH dimer with the tandem VHHs linked C-terminus to N-terminus will invariably produce blocking of CD86 binding.

### Example 9: Functional Analysis of Single-Chain Dimeric Tandem Agents

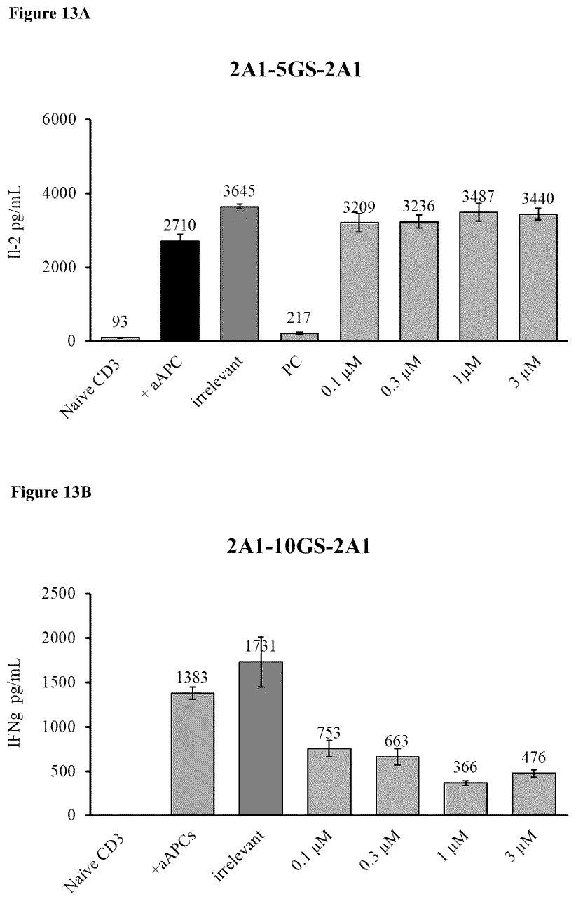

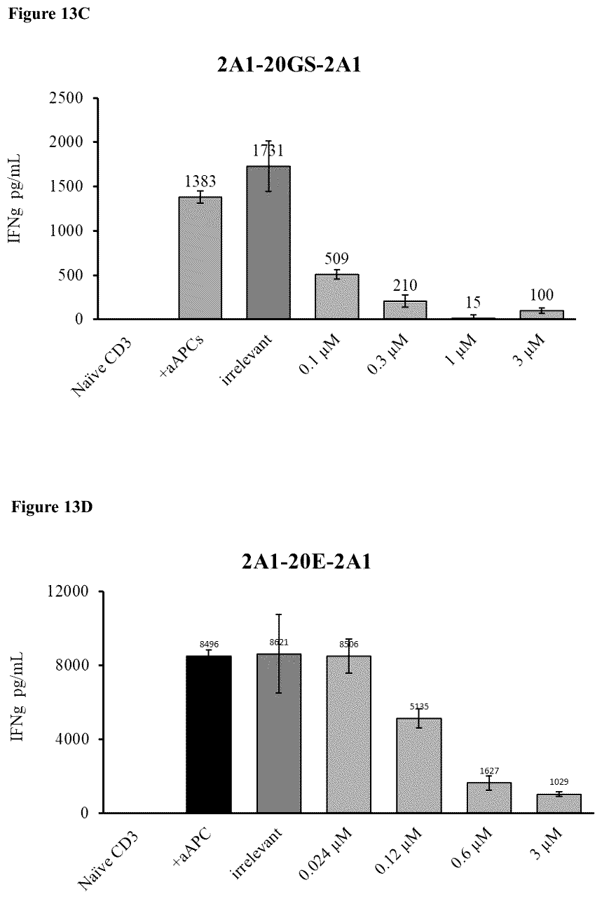

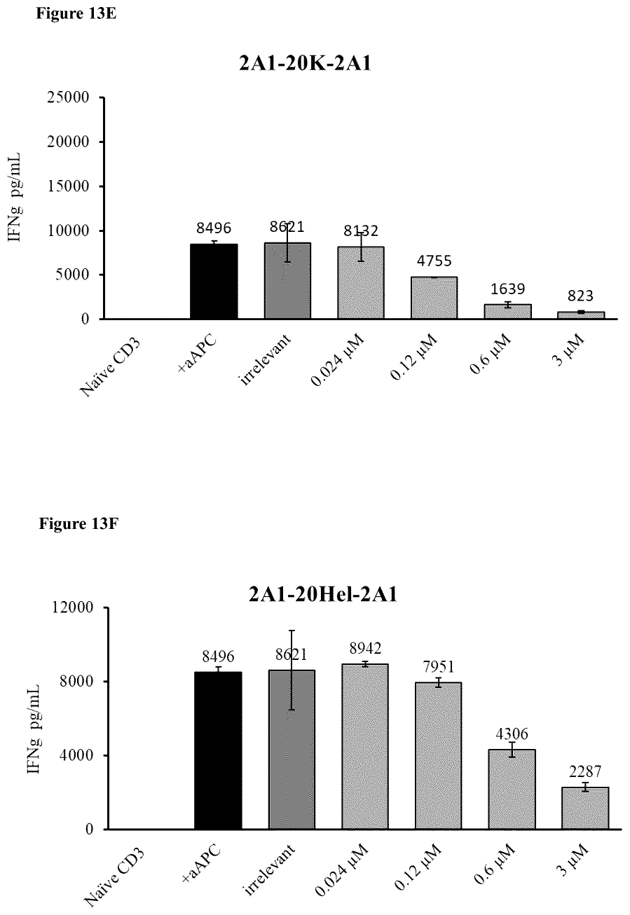

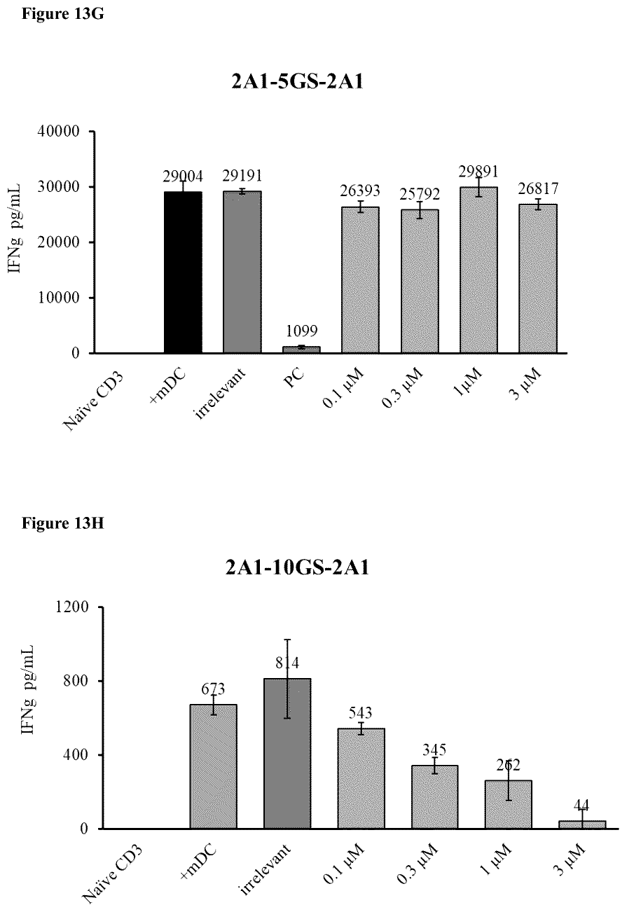

The functional effect of the observed CD86 blocking was further investigated. Isolated T cells were stimulated with artificial antigen presenting cells (aAPCs, HEK cells expressing CD80 and scOKT3) and IL-2 secretion was measured. This was done in the presence of an irrelevant VHH, a VHH known to block CD86 binding and various concentrations of the single-chain dimeric agents (FIG. 13A-13F). As expected, based on the blocking results, the 5GS agent had no effect on IL-2 secretion (FIG. 13A). The 20GS construct, however, nearly completely abolished the induction of IL-2 secretion caused by CD86 even at low concentration (FIG. 13C) and 10GS construct produced an intermediate effect with about 50% reduction in IL-2 secretion at low concentration that increased to about 80% reduction at higher concentrations (FIG. 13B). The three other 20 amino acid linker constructs also produced robust inhibition of IL-2 secretion (FIG. 13D-13F) though the rigid linker construct required higher concentrations in order to produce maximal inhibition (FIG. 13F). Similar results were observed when interferon gamma (IFNg) secretion was measured in a mixed lymphocyte reaction (FIG. 13G-13I). These molecules that produce an antagonistic effect may not be useful for immune stimulation, but maybe employed in conditions of immune overactivation, such as in autoimmune disease.

### Example 10: Generation of Dimeric Agents with Only C-Terminal Linkages

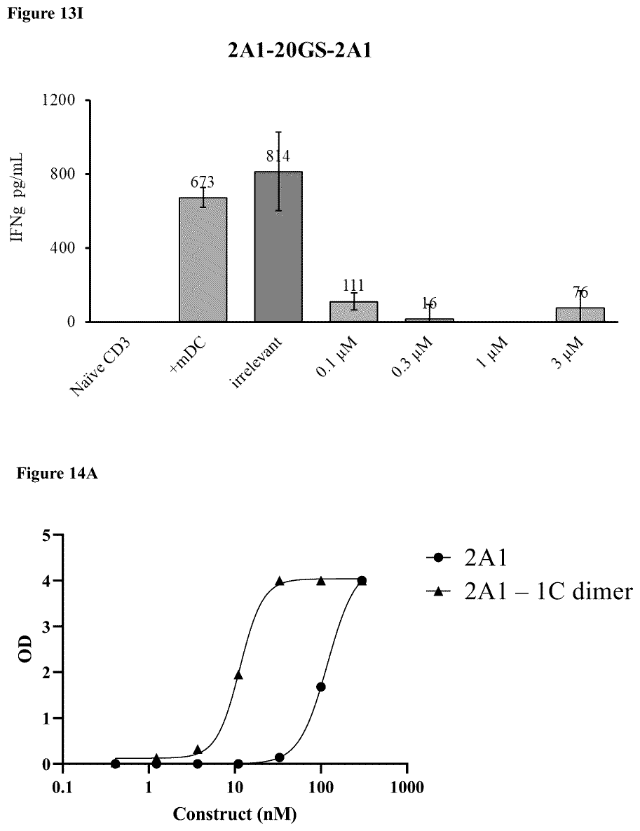

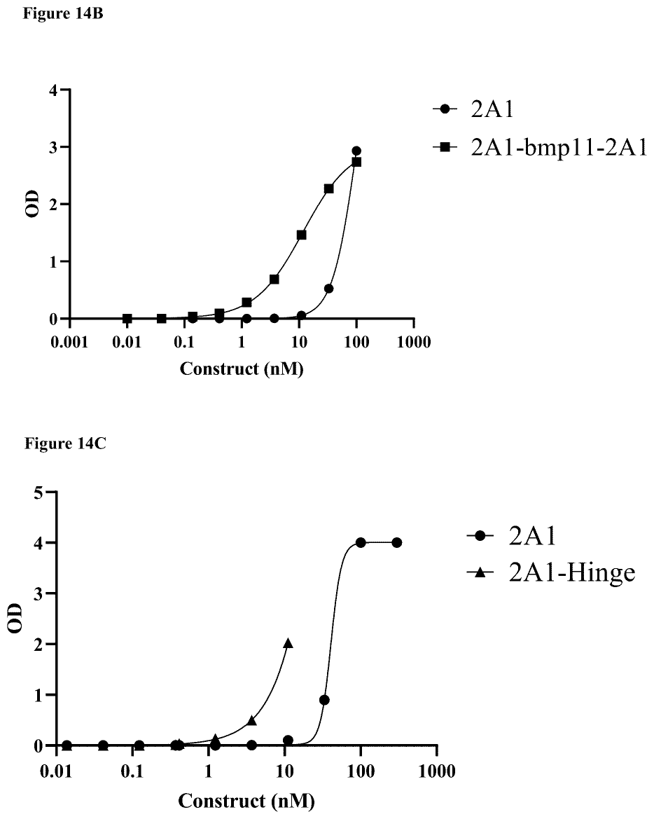

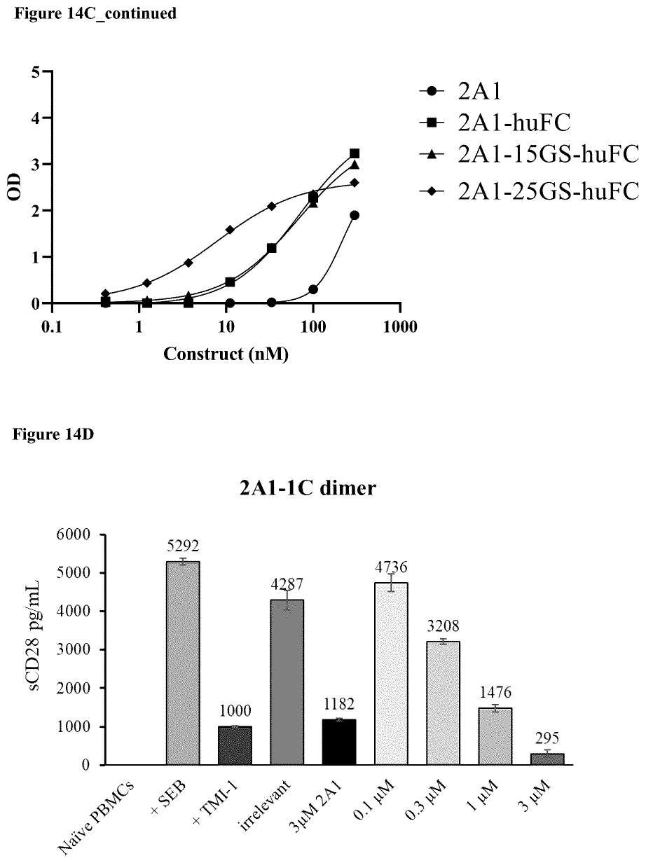

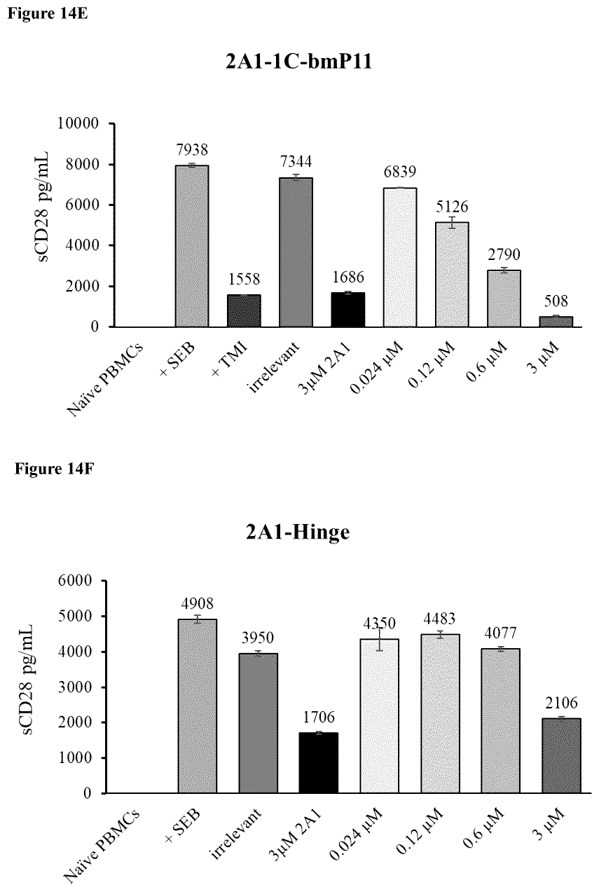

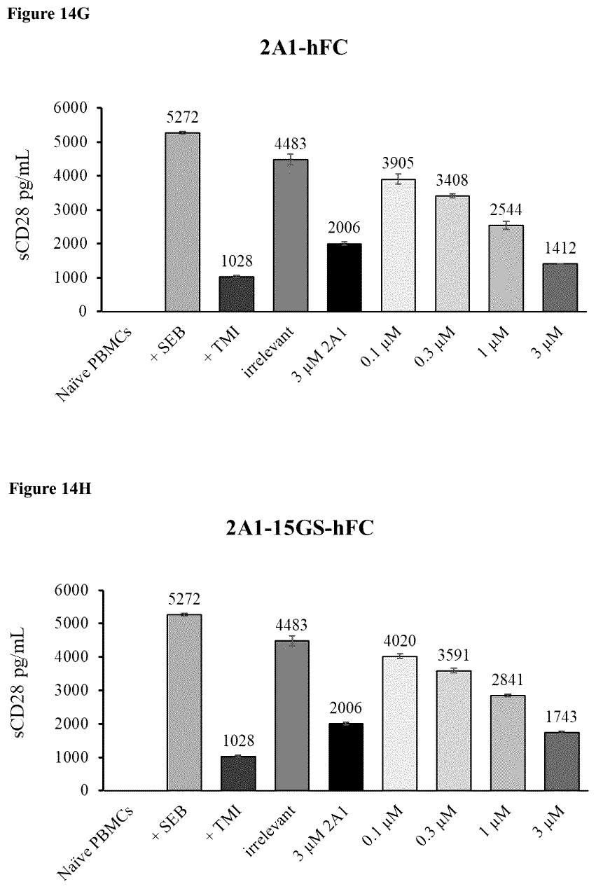

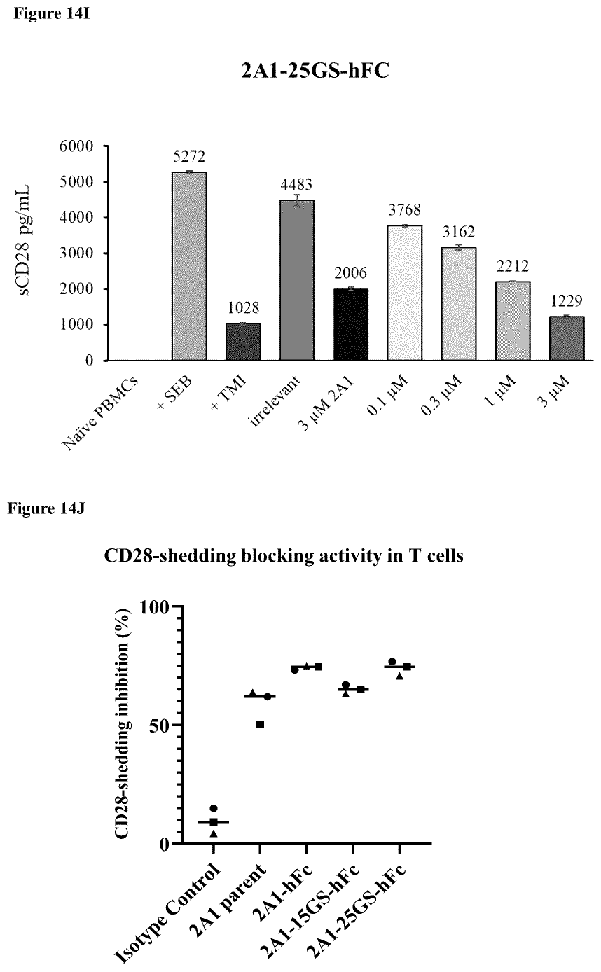

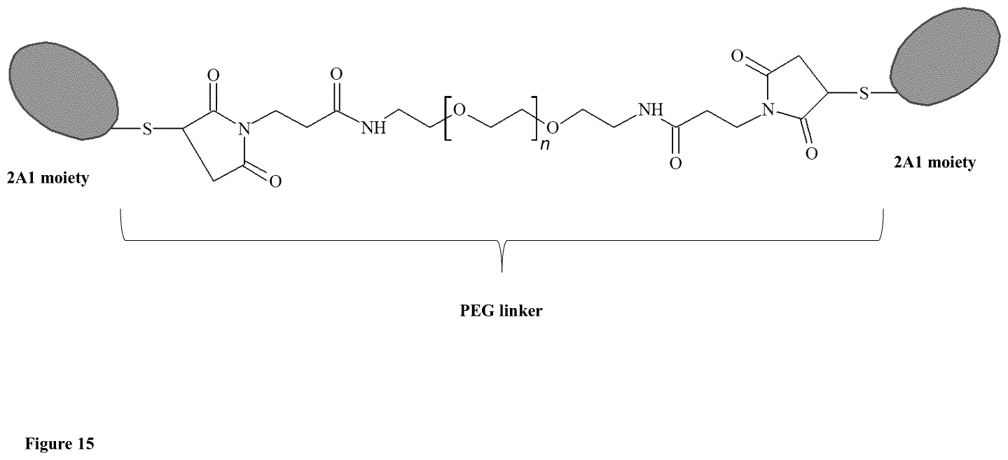

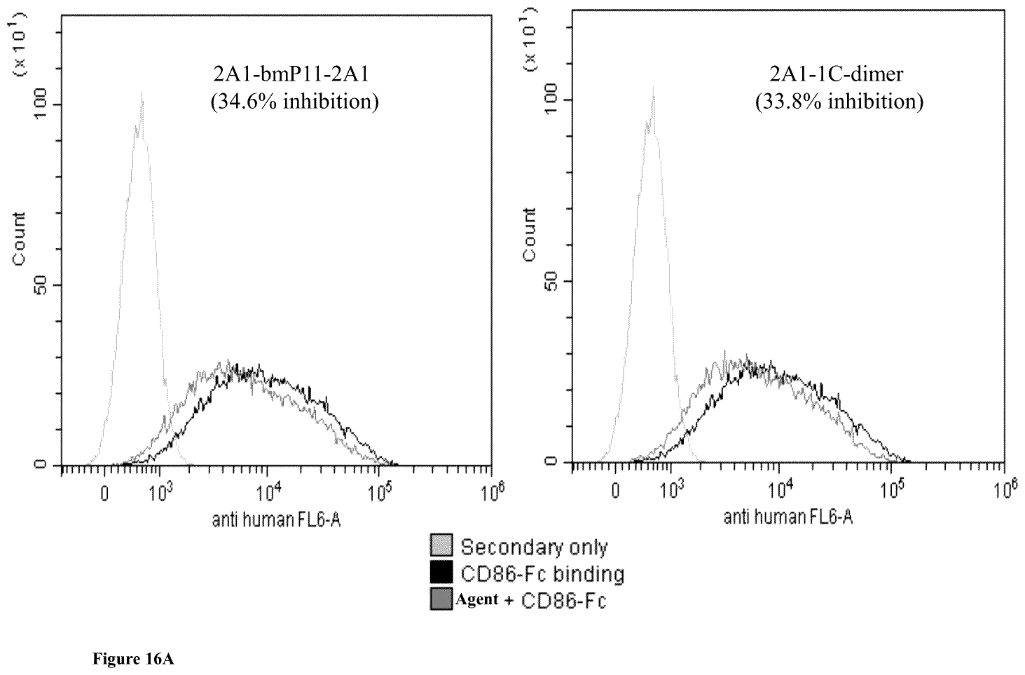

As all of the single-chain agents with the sdAbs linked C-terminus to N-terminus, except for the 5 amino acid linker, produced an antagonistic effect new dimeric agents were designed. For this next set of agents, the linkage was always generated between an area C-terminal to both sdAbs. First a C-terminal cysteine residue was added to the sequence of 2A1 (2A1-C, SEQ ID NO: 113) which was able to dimerize by forming a disulfide bond between the two free cysteines. As desired, a dimeric 2A1-C bound the recombinant CD28-Fc at lower concentrations than the monomeric 2A1 (FIG. 14A). The EC50 values are summarized in Table 13. A similar molecule was also generated with a longer linker. A chemical linker with reactivity towards the free thiol of cysteine (Maleimide) and based on a short discrete PEG chain (PEG₁₁), was used to link the two cysteines (FIG. 15, 2A1-1C-bmp11). This molecule also showed superior binding to CD28 (FIG. 14B). The EC50 values are summarized in Table 14.

TABLE 13
EC50 values for CD28 protein binding of the
1C dimer agent. Values given in nM.

| | | 2A1-1C |
|---|---|---|
| | 2A1 | dimer |
| EC50 | 118.9 | 11.4 |

TABLE 14
EC50 values for CD28 protein binding of the PEG
linker agent. Values given in nM.

| | | 2A1-bmp11 |
|---|---|---|
| | 2A1 | dimer |
| EC50 | 96.8 | 11.81 |

An alternative approach to a C-terminal cysteine is to use a C-terminal dimerization domain. The hinge region of immunoglobulin (Ig) heavy chain has several cysteine residues that are mainly responsible for heavy chain dimerization. So instead of addition of a single cysteine, the human IgG1 hinge region (DKTHTCPPCPAPEL, SEQ ID NO: 38) was inserted downstream of VHH 2A1 (2A1-Hinge, SEQ ID NO: 114). Similarly, the more C-terminal regions of the heavy chain (CH2 and CH3 domains, SEQ ID NO: 39) were also added downstream of the hinge (2A1-huFC, SEQ ID NO: 115). The PG-LALA mutations were incorporated into the heavy chain to reduce effector function. In order to make sure that the Fc did not interfere with VHH binding, two other agents were made with a flexible linker between the VHH and the hinge (2A1-15GS-huFC, SEQ ID NO: 116; 2A1-25GS-huFC, SEQ ID NO: 117). These molecules were all found to improve binding to CD28, with the longer linker producing the best results (FIG. 14C). The EC50 values are summarized in Table 15.

TABLE 15
EC50 values for CD28 protein binding of the human
Fc containing agents. Values given in nM.

| | | | 2A1- | 2A1- |
|---|---|---|---|---|
| | 2A1 | 2A1-hFc | 15GS-hFc | 25GS-hFc |
| EC50 | 214.8 | 70.43 | 68.12 | 7.92 |

As before, the ability of the dimeric agents to inhibit production of sCD28 was also tested. The 2A1-C construct did not inhibit cleavage at its lowest concentration, however, at the same concentration it was found to be superior to the monomeric 2A1 (FIG. 14D). Similarly, the 2A1 with a PEG-based linker also was superior to the monomeric 2A1 when applied at the same concentration (FIG. 14E). The 2A1-hinge agent did inhibit sCD28 production, but only at levels that were comparable to the 2A1 monomer (FIG. 14F). In contrast, the dimeric molecule with the full Fc produced sCD28 inhibition that was comparable to the TMI inhibitor and was superior to the monomeric 2A1 (FIG. 14G). The agents with the flexible linker both performed comparably to the Fc molecule with no linker (FIG. 14H-14I). The improvement in shedding blocking produced by these molecules is summarized in FIG. 14J which depicts results from three different PBMC donors treated with 3 μM of the dimeric agents.

### Example 11: Dimeric Agents with Only C-Terminal Linkages are Less Antagonists

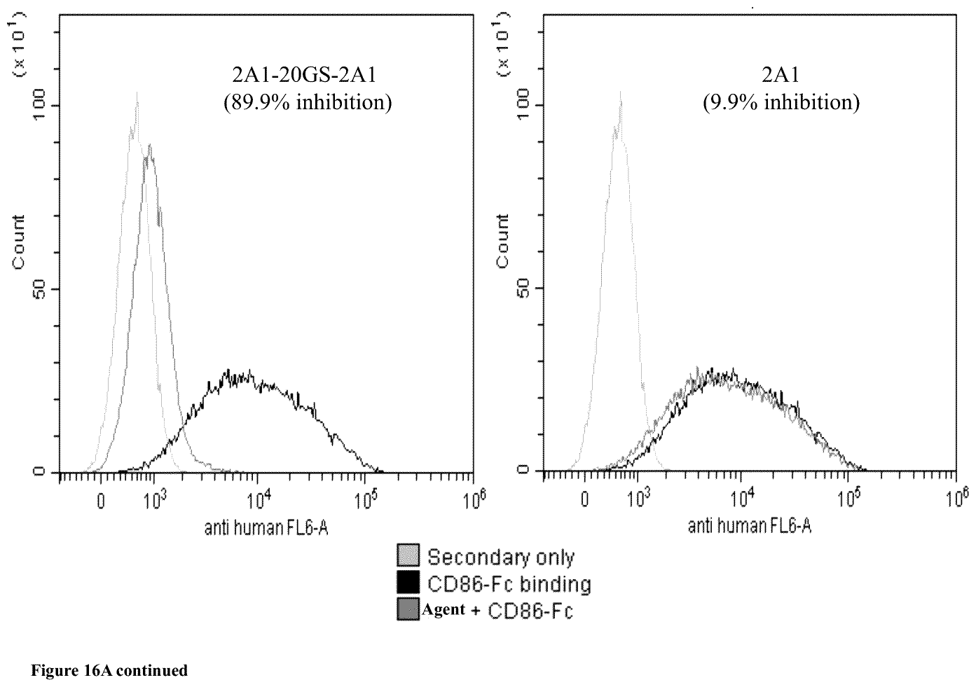

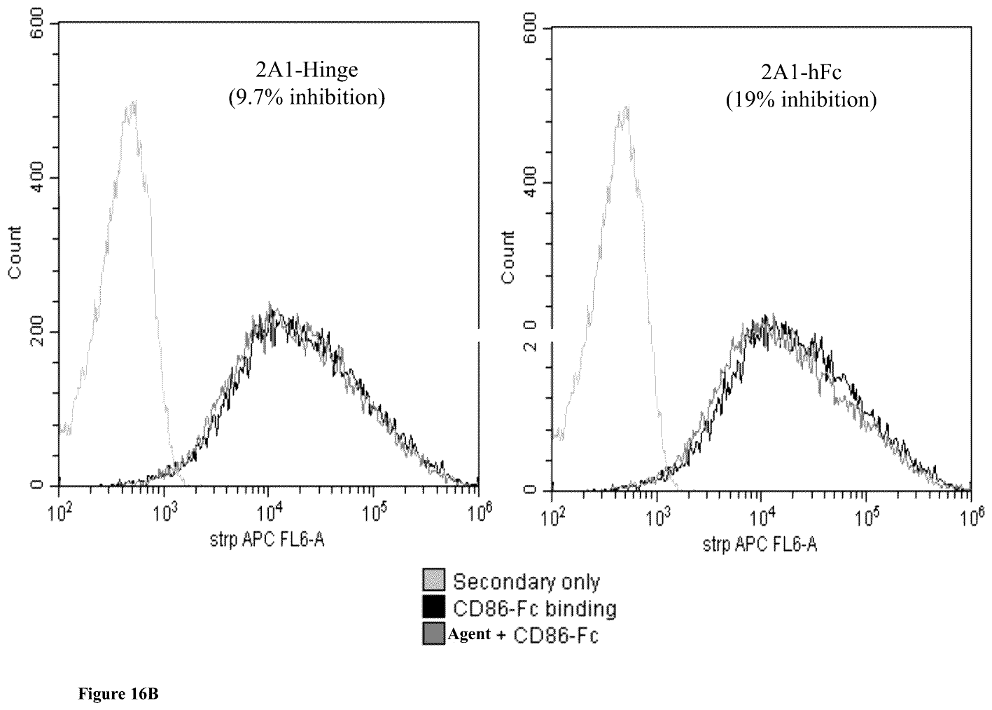

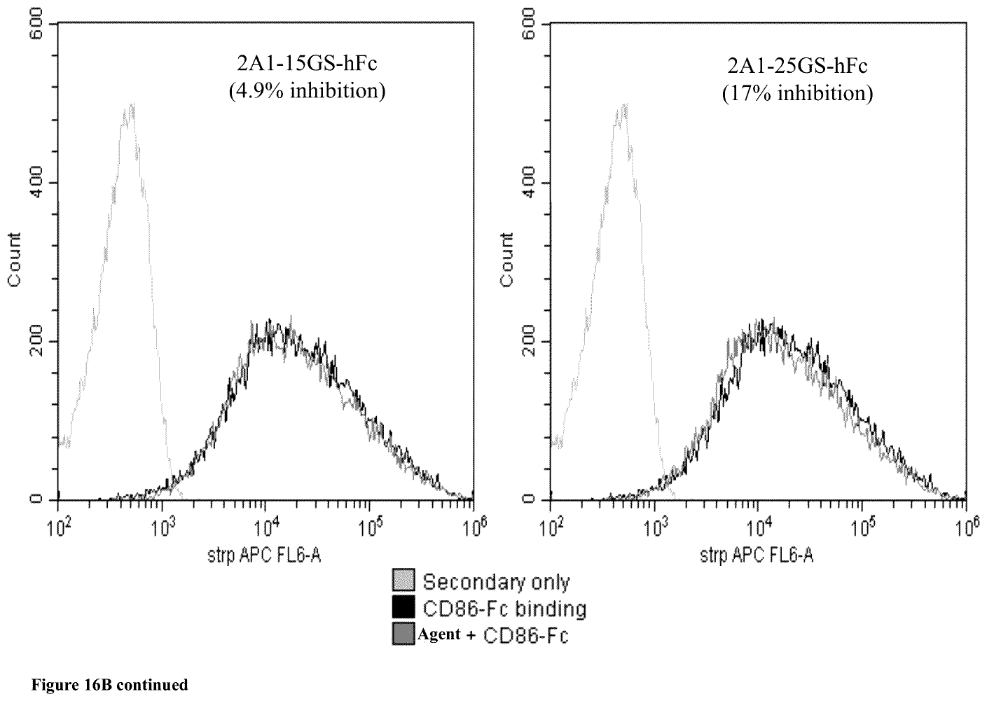

As the first batch of dimeric molecules had unexpectedly been CD28 antagonists, the new batch of dimeric molecules was also tested. The C-terminal molecules whether connected by a disulfide bond or with the PEG based linker, both showed minimal CD86 blocking, but on a level that was vastly superior to the N-C linked dimeric molecules previously tested (FIG. 16A). The molecules that make use of the hinge region for dimerization produced even better results (FIG. 16B). All of these molecules did not significantly inhibit CD86 binding, were comparable to the monomer and thus do not appear to be CD28 antagonists at all. They were further tested for an agonistic effect on CD28, and none was observed (data not shown). All this taken together makes them ideal CD28 shedding blockers, and thus immunostimulatory molecules.

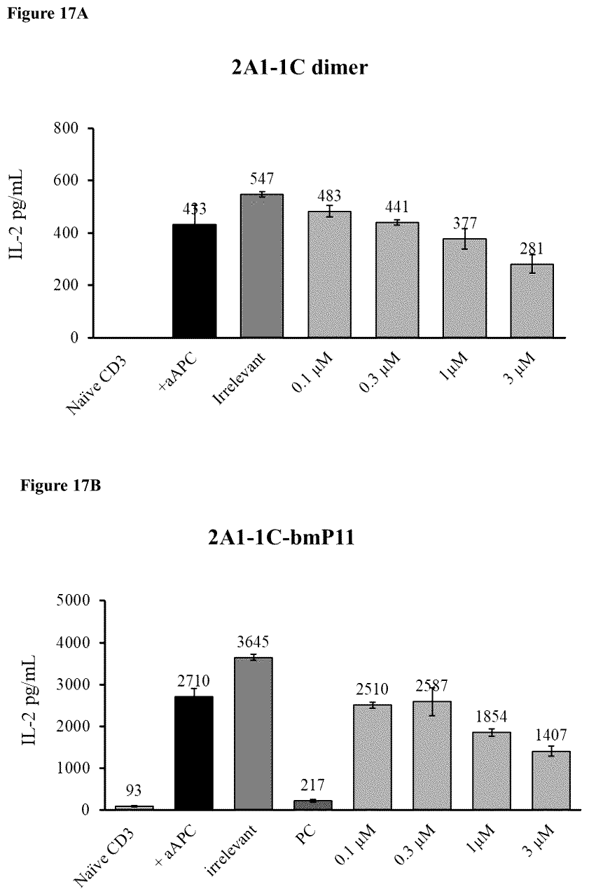

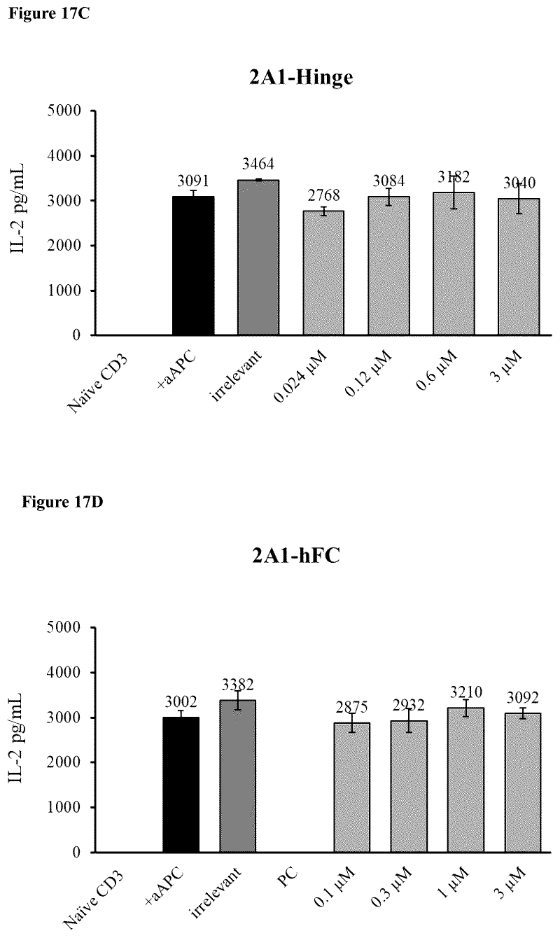

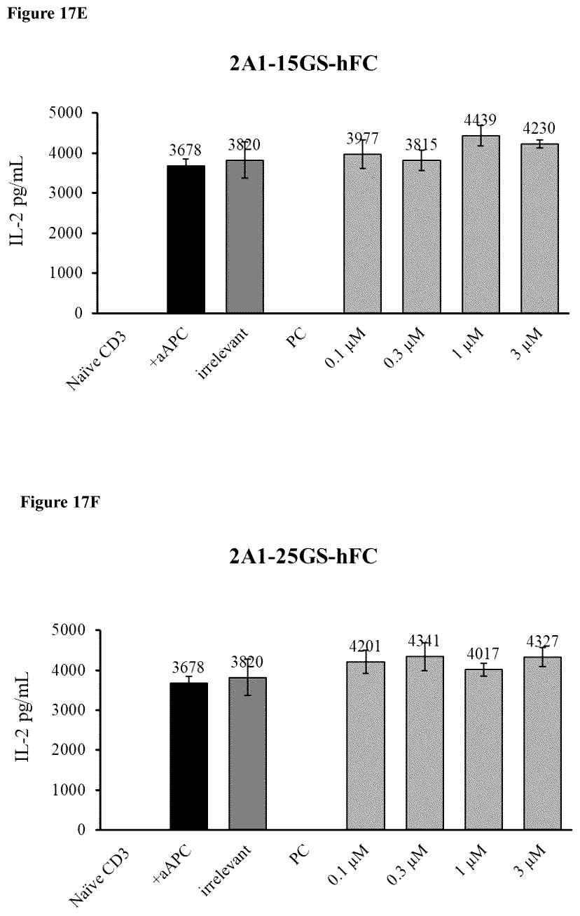

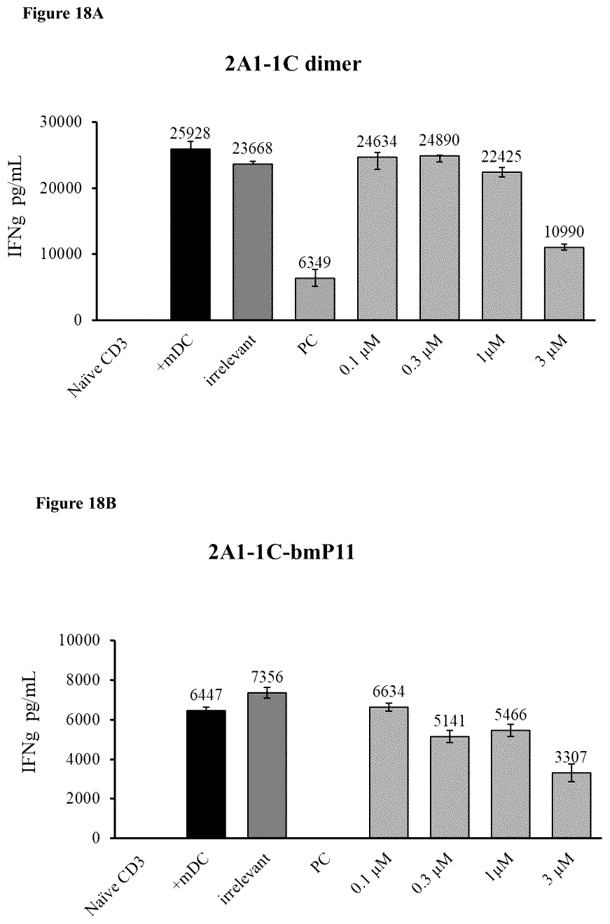

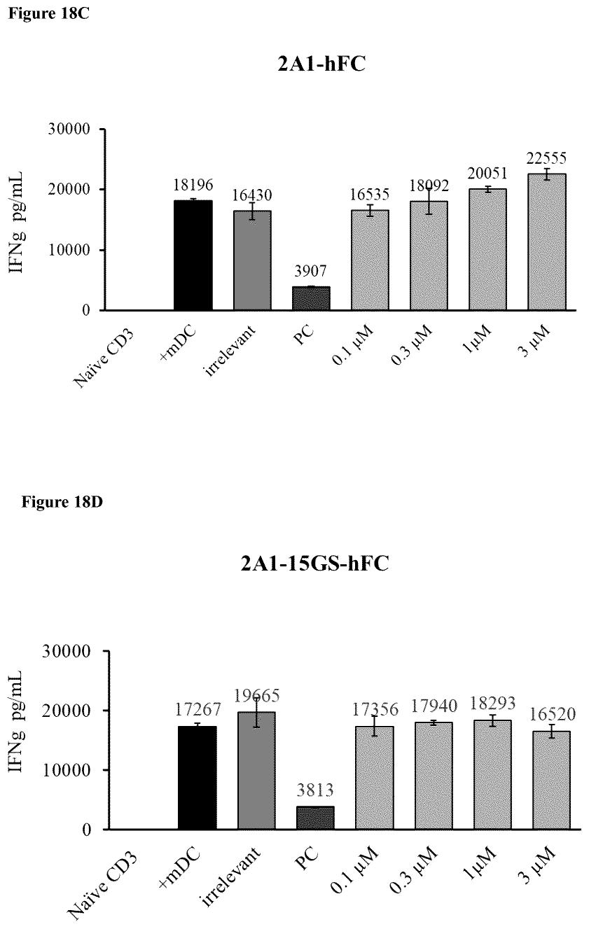

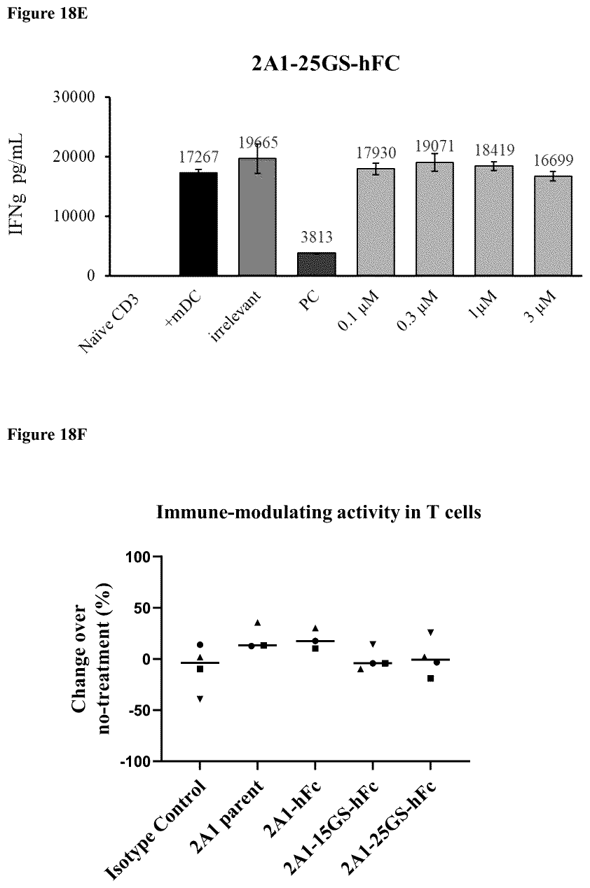

The lack of antagonistic effect was further confirmed in a more physiologically relevant context. As before, secretion of proinflammatory cytokines from T cells was examined with direct aAPC stimulation (FIG. 17A-17F) and in the context of a MLR (FIG. 18A-18E). The 2A1-C dimeric agent produced a mild, dose-dependent reduction in IL-2 levels (FIG. 17A). Similar results were observed in the MLR (FIG. 18A). Use of the PEG-based linker showed similar results, with a mild inhibition observed only at high concentrations (FIGS. 17B and 18B). Once again, the Fc based agents showed no antagonistic effect. The 2A1-Hinge dimeric agent showed no effect on IL-2 secretion (FIG. 17C). The human Fc molecule without a linker also showed no effect on IL-2 secretion (FIG. 17D) and also did not inhibit IFNg secretion (FIG. 18C). The Fc dimeric agents with linkers were just as good with no antagonistic effect whatsoever (FIG. 17E-17F, 18D-18E). The total effect on T-cell activity is summarized in FIG. 18F, which shows results from MLR assay with cells from four different donors. The molecules that made use of C-terminal linkers, and in particular those with a dimerization domain such as the hinge or Fc, were all successful in not producing a substantial antagonistic effect while also blocking cleavage, making them ideal for immune stimulation in the context of excess sCD28 production.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A single domain antibody (sdAb) comprising three CDRs wherein:
CDR1 comprises the amino acid sequence set forth in SEQ ID NO: 1 (INSMG), CDR2 comprises the amino acid sequence as set forth in SEQ ID NO: 2 (AINEKLLIYYADSVKG), CDR3 comprises the amino acid sequence as set forth in SEQ ID NO: 3 (DLYGSDYWD);
CDR1 comprises the amino acid sequence set forth in SEQ ID NO: 4 (INAMG), CDR2 comprises the amino acid sequence as set forth in SEQ ID NO: 5 (AISGGGDTYYADSVKG), CDR3 comprises the amino acid sequence as set forth in SEQ ID NO: 6 (DMIEQQWWY);
CDR1 comprises the amino acid sequence set forth in SEQ ID NO: 4 (INAMG), CDR2 comprises the amino acid sequence as set forth in SEQ ID NO: 5 (AISGGGDTYYADSVKG), CDR3 comprises the amino acid sequence as set forth in SEQ ID NO: 7 (DTHRGVYWY);
CDR1 comprises the amino acid sequence set forth in SEQ ID NO: 8 (IKTMA), CDR2 comprises the amino acid sequence as set forth in SEQ ID NO: 9 (AINYIKEIYYADSVKG), CDR3 comprises the amino acid sequence as set forth in SEQ ID NO: 10 (DVTKEDYWY);
CDR1 comprises the amino acid sequence set forth in SEQ ID NO: 11 (INSMA), CDR2 comprises the amino acid sequence as set forth in SEQ ID NO: 12 (AISNAREVYYADSVKG), CDR3 comprises the amino acid sequence as set forth in SEQ ID NO: 13 (DVYFQEYWY);
CDR1 comprises the amino acid sequence set forth in SEQ ID NO: 14 (INTMA), CDR2 comprises the amino acid sequence as set forth in SEQ ID NO: 15 (AINSISRTYYADSVKG), CDR3 comprises the amino acid sequence as set forth in SEQ ID NO: 10 (DVTKEDYWY);
CDR1 comprises the amino acid sequence set forth in SEQ ID NO: 8 (IKTMA), CDR2 comprises the amino acid sequence as set forth in SEQ ID NO: 16 (AIASDNRKYYADSVKG), CDR3 comprises the amino acid sequence as set forth in SEQ ID NO: 10 (DVTKEDYWY);
CDR1 comprises the amino acid sequence set forth in SEQ ID NO: 17 (IRTMA), CDR2 comprises the amino acid sequence as set forth in SEQ ID NO: 18 (AISSGREVYYADSVKG), CDR3 comprises the amino acid sequence as set forth in SEQ ID NO: 19 (DMYWQDYWW); or
CDR1 comprises the amino acid sequence set forth in SEQ ID NO: 1 (INSMG), CDR2 comprises the amino acid sequence as set forth in SEQ ID NO: 20 (AISDRSEKYYADSVKG), CDR3 comprises the amino acid sequence as set forth in SEQ ID NO: 21 (DHHHSDWWT).

2. The sdAb of claim 1, wherein said sdAb is a camelid or shark antibody.

3. (canceled)

4. The sdAb of claim 1, wherein
a. a sequence N-terminal to CDR1 consists of X₁VQLVESGGGLVQX₂GX₃SLRLSCX₄ASGSX₅X₆S (SEQ ID NO: X), wherein X₁ is E or Q, X₂ is A or P, X₃ is E or G, X₄ is A or K, X₅ is I, L or T and X₆ is A or F; a sequence between CDR1 and CDR2 consists of WYRQAPGX₇X₈X₉EX₁₀VX₁₁ (SEQ ID NO: X), wherein X₇ is S or K, X₈ is Q or G, X₉ is R or L, X₁₀ is L or R, and X₁₁ is one of: A, S, or T; a sequence between CDR2 and CDR3 consists of RFTX₁₁SRDNX₁₂KX₁₃TX₁₄YLQMNX₁₅LX₁₆X₁₇X₁₈DX₁₉X₂₀VYYCVV (SEQ ID NO: X), wherein X₁₁ is I or V, X₁₂ is A or S, X₁₃ is T or N, X₁₄ is V, M or L, X₁₅ is S or N, X₁₆ is R, K, or E, and X₁₇ is P or A, X₁₈ is E or R, X₁₉ is T or A, X₂₀ is A or G; and a sequence C-terminal to CDR3 consists of WGQGTX₂₁VTVSS (SEQ ID NO: X), wherein X₂₁ is an Q or L; or
b. a sequence N-terminal to CDR1 consists of EVQLVESGGGLVQAGESLRLSCAASGSIAS (SEQ ID NO: 22), a sequence between CDR1 and CDR2 consists of WYRQAPGSQRELVX (SEQ ID NO: 48), a sequence between CDR2 and CDR3 consists of RFTISRDNAKTTVYLQMNSLRPEDTAVYYCVV (SEQ ID NO: 24) and a sequence C-terminal to CDR3 consists of WGQGTQVTVSS (SEQ ID NO: 25), wherein X is an A or T.

5. (canceled)

6. The sdAb of claim 1, wherein said sdAb comprises a sequence selected from a group consisting of:
a.
(SEQ ID NO: 26)
EVQLVESGGGLVQAGESLRLSCAASGSIASINSMGWYRQAPGSQR
ELVAAINEKLLIYYADSVKGRFTISRDNAKTTVYLQMNSLRPEDTA
VYYCVVDLYGSDYWDWGQGTQVTVSS;
b.
(SEQ ID NO: 27)
EVQLVESGGGLVQAGESLRLSCAASGSIASINAMGWYRQAPGSQR
ELVAAISGGGDTYYADSVKGRFTISRDNAKTTVYLQMNSLRPEDT
AVYYCVVDMIEQQWWYWGQGTQVTVSS;
c.
(SEQ ID NO: 28)
EVQLVESGGGLVQAGESLRLSCAASGSIASINAMGWYRQAPGSQR
ELVAAISGGGDTYYADSVKGRFTISRDNAKTTVYLQMNSLRPEDT
AVYYCVVDTHRGVYWYWGQGTQVTVSS;
d.
(SEQ ID NO: 29)
EVQLVESGGGLVQAGESLRLSCAASGSIASIKTMAWYRQAPGSQR
ELVAAINYIKEIYYADSVKGRFTISRDNAKTTVYLQMNSLRPEDTA
VYYCVVDVTKEDYWYWGQGTQVTVSS;
e.
(SEQ ID NO: 30)
EVQLVESGGGLVQAGESLRLSCAASGSIASINSMAWYRQAPGSQR
ELVAAISNAREVYYADSVKGRFTISRDNAKTTVYLQMNSLRPEDT
AVYYCVVDVYFQEYWYWGQGTQVTVSS;
f.
(SEQ ID NO: 31)
EVQLVESGGGLVQAGESLRLSCAASGSIASINTMAWYRQAPGSQR
ELVAAINSISRTYYADSVKGRFTISRDNAKTTVYLQMNSLRPEDTA
VYYCVVDVTKEDYWYWGQGTQVTVSS;
g.
(SEQ ID NO: 32)
EVQLVESGGGLVQAGESLRLSCAASGSIASIKTMAWYRQAPGSQR
ELVTAIASDNRKYYADSVKGRFTISRDNAKTTVYLQMNSLRPEDT
AVYYCVVDVTKEDYWYWGQGTQVTVSS;
h.
(SEQ ID NO: 70)
EVQLVESGGGLVQPGGSLRLSCAASGSIASIKTMAWYRQAPGKQR
ELVTAIASDNRKYYADSVKGRFTISRDNSKNTLYLQMNSLRAEDT
AVYYCVVDVTKEDYWYWGQGTLVTVSS;
i.
(SEQ ID NO: 71)
EVQLVESGGGLVQPGGSLRLSCKASGSIASIKTMAWYRQAPGKGL
ELVTAIASDNRKYYADSVKGRFTISRDNSKTTVYLQMNSLRAEDT
AVYYCVVDVTKEDYWYWGQGTLVTVSS;
j.
(SEQ ID NO: 72)
EVQLVESGGGLVQPGGSLRLSCAASGSTASIKTMAWYRQAPGKG
LELVTAIASDNRKYYADSVKGRFTISRDNSKTTVYLQMNSLRAED
TAVYYCVVDVTKEDYWYWGQGTLVTVSS;
k.
(SEQ ID NO: 73)
EVQLVESGGGLVQPGGSLRLSCKASGSTASIKTMAWYRQAPGKG
LELVTAIASDNRKYYADSVKGRFTISRDNSKTTVYLQMNSLRAED
TAVYYCVVDVTKEDYWYWGQGTLVTVSS;
l.
(SEQ ID NO: 74)
EVQLVESGGGLVQPGGSLRLSCAASGSIASIKTMAWYRQAPGKGR
ELVTAIASDNRKYYADSVKGRFTISRDNSKTTVYLQMNSLRAEDT
AVYYCVVDVTKEDYWYWGQGTLVTVSS;
m.
(SEQ ID NO: 33)
EVQLVESGGGLVQAGESLRLSCAASGSIASIRTMAWYRQAPGSQR
ELVAAISSGREVYYADSVKGRFTISRDNAKTTVYLQMNSLRPEDT
AVYYCVVDMYWQDYWWWGQGTQVTVSS;
n.
(SEQ ID NO: 75)
EVQLVESGGGLVQPGESLRLSCAASGSIASIRTMAWYRQAPGSQR
ELVAAISSGREVYYADSVKGRFTISRDNAKTTVYLQMNSLRAEDT
AVYYCVVDMYWQDYWWWGQGTQVTVSS;
o.
(SEQ ID NO: 76)
EVQLVESGGGLVQPGGSLRLSCKASGSIASIRTMAWYRQAPGKGL
ELVAAISSGREVYYADSVKGRFTISRDNSKTTVYLQMNSLRAEDT
AVYYCVVDMYWQDYWWWGQGTLVTVSS;
p.
(SEQ ID NO: 77)
EVQLVESGGGLVQPGGSLRLSCKASGSTASIRTMAWYRQAPGKGL
ELVSAISSGREVYYADSVKGRFTISRDNSKTTVYLQMNSLRAEDTA
VYYCVVDMYWQDYWWWGQGTLVTVSS;
q.
(SEQ ID NO: 78)
EVQLVESGGGLVQPGGSLRLSCAASGSIASIRTMAWYRQAPGKGL
ELVSAISSGREVYYADSVKGRFTISRDNSKTTVYLQMNSLRAEDTA
VYYCVVDMYWQDYWWWGQGTLVTVSS;
or
r.
(SEQ ID NO: 34)
EVQLVESGGGLVQAGESLRLSCAASGSIASINSMGWYRQAPGSQR
ELVAAISDRSEKYYADSVKGRFTISRDNAKTTVYLQMNSLRPEDT
AVYYCVVDHHHSDWWTWGQGTQVTVSS.

7. The sdAb of claim 1, wherein at least one of:
a. said sdAb is not a CD28 agonist;
b. said sdAb is not a CD28 antagonist;
c. said sdAb neither degrades said mCD28 nor inhibits mCD28-mediated immune cell activation;
d. said sdAb binds within the stalk region of CD28; and
e. said sdAb bins the amino acid sequence GKHLCPSPLFPGPSKP (SEQ ID NO: 35) or KGKHLCPSPLFPGPS (SEQ ID NO: 36) of CD28.

8. (canceled)

9. (canceled)

10. (canceled)

11. (canceled)

12. (canceled)

13. (canceled)

14. (canceled)

15. (canceled)

16. (canceled)

17. (canceled)

18. A dimeric agent comprising at least two membranal CD28 (mCD28) binding single domain antibodies (sdAbs), wherein a first mCD28 binding sdAb is linked to a second mCD28 binding sdAb by a linker.

19. (canceled)

20. A dimeric agent comprising at least two membranal CD28 (mCD28) binding single domain antibodies (sdAbs), wherein a first mCD28 binding sdAb is linked to a second mCD28 binding sdAb by a linker, and comprising a sdAb of claim 1.

21. The dimeric agent of claim 20, wherein at least one of:
a. said first sdAb and said second sdAb comprise the same sequence;
b. said first sdAb, said second sdAb or both when not part of a dimeric agent is a CD28 antagonist and wherein said dimeric agent is not a CD28 antagonist;
c. said dimeric agent comprises a first polypeptide comprising said first sdAb and a second polypeptide comprising said second sdAb and wherein said linker links said first polypeptide and said second polypeptide;
d. said dimeric agent comprises a first polypeptide comprising said first sdAb and a first free cysteine amino acid outside of said first sdAb and a second polypeptide comprising said second sdAb and a second free cysteine amino acid outside of said second sdAb and wherein said linker comprises a bond between said first free cysteine and said second free cysteine;
e. wherein said dimeric agent comprises a first polypeptide comprises a first polypeptide comprising said first sdAb and a first dimerization domain, and a second polypeptide comprising said second sdAb and a second dimerization domain and wherein said linker comprises said dimerization domains, a bond between said dimerization domains or both
f. wherein said linker is an amino acid linker;
g. wherein said linker is a chemical linker;
h. wherein said linker comprises a net neutral charge;
i. said amino acid linker comprises (a sequence selected from (GGGGS)n, (GS)n, (GGS)n, (GSGGS)n, and a combination thereof, wherein n is an integer selected from 1, 2, 3, 4, 5, 6, 7, and 8;
j. wherein said linker comprises a net positive charge;
k. wherein said amino acid linker comprises a sequence selected from (XGGGS)n, (XGGS)n, (GGGXS)n, and a combination thereof, wherein X is selected from K, R and H and n is an integer selected from 1, 2, 3, 4, 5, 6, 7 and 8;
l. wherein said linker is an amino acid linker comprising
(SEQ ID NO: 97)
GGGGSAEAAAKEAAAKEAAAKAAAGSGGGGS;
m. wherein said amino acid linker comprises at most 100 amino acids.

22. (canceled)

23. The dimeric agent of claim 20, wherein said dimeric agent inhibits proteolytic cleavage of said mCD28.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. The dimeric agent of claim 3820, wherein said dimeric agent comprises a first polypeptide comprises a first polypeptide comprising said first sdAb and a first dimerization domain, and a second polypeptide comprising said second sdAb and a second dimerization domain and wherein said dimerization domain comprises DKTHTCPPCPAPEAAGGPSVFLFPPKPKDTLMISRTPEVTCVVVDVSHEDP EVKFNWYVDGVEVHNAKTKPREEQYNSTYRVVSVLTVLHQDWLNGKE YKCKVSNKALGAPIEKTISKAKGQPREPQVYTLPPSRDELTKNQVSLTCL VKGFYPSDIAVEWESNGQPENNYKTTPPVLDSDGSFFLYSKLTVDKSRWQ QGNVFSCSVMHEALHNHYTQKSLSLSPGK (SEQ ID NO: 39), er ESKYGPPCPPCPAPEFEGGPSVFLFPPKPKDTLMISRTPEVTCVVVDVSQE DPEVQFNWYVDGVEVHNAKTKPREEQFNSTYRVVSVLTVLHQDWLNGK EYKCKVSNKGLPSSIEKTISKAKGQPREPQVYTLPPSQEEMTKNQVSLTCL VKGFYPSDIAVEWESNGQPENNYKTTPPVLDSDGSFFLYSRLTVDKSRWQ EGNVFSCSVMHEALHNHYTQKSLSLSLGK (SEQ ID NO: 92) or a sequence with at least 95% identity thereto.

40. (canceled)

41. (canceled)

42. (canceled)

43. (canceled)

44. (canceled)

45. (canceled)

46. (canceled)

47. The dimeric agent of claim 20, comprising a single polypeptide, wherein said single polypeptide comprises said first sdAb N-terminal to said second sdAb and separated by an amino acid linker of fewer than 13 amino acids, wherein said dimeric agent is not a CD28 antagonist, inhibits ligand binding to CD28 by less than 50%, or both or separated by an amino acid linker of equal to or greater than 10 amino acids and wherein said dimeric agent is a CD28 antagonist.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. A method of decreasing soluble CD28 (sCD28) levels, treating and/or preventing cancer, improving PD-1 and/or PD-L1 based immunotherapy against cancer or in a subject in need thereof, the method comprising administering to said subject a dimeric agent of claim 20, thereby decreasing sCD28 levels, treating and/or preventing cancer or improving PD-1/PD-L1 based immunotherapy against cancer.

63. (canceled)

64. (canceled)

65. (canceled)

66. The method of claim 62, wherein said cancer is selected from melanoma, head and neck, non-small cell lung cancer, ovarian, kidney, gastric and colorectal.

67. (canceled)

68. (canceled)

69. (canceled)

70. (canceled)

71. A method for suppressing an immune response in a subject in need thereof, the method comprising administering to said subject a sdAb of claim 1, thereby suppressing an immune response.

72. (canceled)

73. (canceled)

74. The method of claim 71, wherein said subject is afflicted with an autoimmune disease, optionally wherein said autoimmune disease is selected from the group consisting of: lupus, rheumatoid arthritis, Crohn's disease, inflammatory bowel disease, Becht's disease, colitis, ulcerative colitis, diabetes, Graves' disease, and multiple sclerosis.

75. (canceled)

76. A pharmaceutical composition comprising a sdAb of claim 1 and a pharmaceutical acceptable carrier, excipient or adjuvant.

77. (canceled)

78. (canceled)

79. A kit comprising at least one dimeric agent of claim 20 and at least one of:
a. an anti-PD-1 and/or PD-L1 immunotherapy; and
b. a label stating the agent of the invention is for use with a PD-1 and/or PD-L1 based immunotherapy.

80. (canceled)

81. A method of generating a dimeric agent that inhibits proteolytic cleavage of mCD28 on a surface of a cell, comprising at least one of:
a. i. obtaining an agent that binds to mCD28 on a cell surface and blocks cleavage of said mCD28 by a protease;
ii. linking a first moiety of said agent to a second moiety of said agent via a linker to produce a dimeric agent;
iii. testing an ability of said dimeric agent to block cleavage of mCD28 on a cell surface by a protease; and
iv. selecting a dimeric agent that blocks cleavage of mCD28 on a cell surface;
and
b. culturing a host cell comprising one or more vectors comprising one or more nucleic acid sequences encoding a dimeric agent, wherein the one or more nucleic acid sequences are that of a dimeric agent that was selected by:
i. obtaining an agent that binds to mCD28 on a cell surface and blocks cleavage of said mCD28 by a protease, optionally wherein said obtained agent is a sdAb;
ii. linking a first moiety of said agent to a second moiety of said agent via a linker to produce a dimeric agent;
iii. testing an ability of said dimeric agent to block cleavage of mCD28 on a cell surface by a protease; and
iv. selecting an agent that blocks cleavage of mCD28 on a cell surface;
and optionally further comprising assaying mCD28 downstream signaling in the presence of said obtained dimeric agent and selecting at least one dimeric agent that either a) neither substantially agonizes nor substantially antagonizes mCD28 signaling or b) substantially antagonizes mCD28 signaling; thereby generating an agent that inhibits proteolytic cleavage of mCD28 on a surface of a cell.

82. (canceled)

83. (canceled)

84. (canceled)

85. (canceled)

86. (canceled)

87. (canceled)

88. (canceled)

89. (canceled)

90. (canceled)

91. (canceled)

92. The dimeric agent of claim 39, comprising an sdAb consisting of SEQ ID NO: 74, a dimerization domain consisting of SEQ ID NO: 92 or a sequence with at least 95% identity thereto and a linker linking said sdAb to said dimerization domain, wherein said linker comprises five repeats of SEQ ID NO: 118.

93. The dimeric agent of claim 92, consisting from N-terminus to C-terminus of SEQ ID NO: 74, five repeats of SEQ ID NO: 118 and SEQ ID NO: 92.


SEQUENCE LISTING (119 sequences)

SEQ ID NO: 1 - protein, 5 aa, synthetic construct
  REGION 1..5: note = Synthetic
INSMG

SEQ ID NO: 2 - protein, 16 aa, synthetic construct
  REGION 1..16: note = Synthetic
AINEKLLIYYADSVKG

SEQ ID NO: 3 - protein, 9 aa, synthetic construct
  REGION 1..9: note = Synthetic
DLYGSDYWD

SEQ ID NO: 4 - protein, 5 aa, synthetic construct
  REGION 1..5: note = Synthetic
INAMG

SEQ ID NO: 5 - protein, 16 aa, synthetic construct
  REGION 1..16: note = Synthetic
AISGGGDTYYADSVKG

SEQ ID NO: 6 - protein, 9 aa, synthetic construct
  REGION 1..9: note = Synthetic
DMIEQQWWY

SEQ ID NO: 7 - protein, 9 aa, synthetic construct
  REGION 1..9: note = Synthetic
DTHRGVYWY

SEQ ID NO: 8 - protein, 5 aa, synthetic construct
  REGION 1..5: note = Synthetic
IKTMA

SEQ ID NO: 9 - protein, 16 aa, synthetic construct
  REGION 1..16: note = Synthetic
AINYIKEIYYADSVKG

SEQ ID NO: 10 - protein, 9 aa, synthetic construct
  REGION 1..9: note = Synthetic
DVTKEDYWY

SEQ ID NO: 11 - protein, 5 aa, synthetic construct
  REGION 1..5: note = Synthetic
INSMA

SEQ ID NO: 12 - protein, 16 aa, synthetic construct
  REGION 1..16: note = Synthetic
AISNAREVYYADSVKG

SEQ ID NO: 13 - protein, 9 aa, synthetic construct
  REGION 1..9: note = Synthetic
DVYFQEYWY

SEQ ID NO: 14 - protein, 5 aa, synthetic construct
  REGION 1..5: note = Synthetic
INTMA

SEQ ID NO: 15 - protein, 16 aa, synthetic construct
  REGION 1..16: note = Synthetic
AINSISRTYYADSVKG

SEQ ID NO: 16 - protein, 16 aa, synthetic construct
  REGION 1..16: note = Synthetic
AIASDNRKYYADSVKG

SEQ ID NO: 17 - protein, 5 aa, synthetic construct
  REGION 1..5: note = Synthetic
IRTMA

SEQ ID NO: 18 - protein, 16 aa, synthetic construct
  REGION 1..16: note = Synthetic
AISSGREVYYADSVKG

SEQ ID NO: 19 - protein, 9 aa, synthetic construct
  REGION 1..9: note = Synthetic
DMYWQDYWW

SEQ ID NO: 20 - protein, 16 aa, synthetic construct
  REGION 1..16: note = Synthetic
AISDRSEKYYADSVKG

SEQ ID NO: 21 - protein, 9 aa, synthetic construct
  REGION 1..9: note = Synthetic
DHHHSDWWT

SEQ ID NO: 22 - protein, 30 aa, synthetic construct
  REGION 1..30: note = Synthetic
EVQLVESGGGLVQAGESLRLSCAASGSIAS

SEQ ID NO: 23 - protein, 14 aa, synthetic construct
  REGION 1..14: note = Synthetic
WYRQAPGSQRELVA

SEQ ID NO: 24 - protein, 32 aa, synthetic construct
  REGION 1..32: note = Synthetic
RFTISRDNAKTTVYLQMNSLRPEDTAVYYCVV

SEQ ID NO: 25 - protein, 11 aa, synthetic construct
  REGION 1..11: note = Synthetic
WGQGTQVTVSS

SEQ ID NO: 26 - protein, 117 aa, synthetic construct
  REGION 1..117: note = Synthetic
EVQLVESGGGLVQAGESLRLSCAASGSIASINSMGWYRQAPGSQRELVAAINEKLLIYYADSVKGRFTISRDNAKTTVYLQMNSLRPEDTAVYYCVVDLYGSDYWDWGQGTQVTVSS

SEQ ID NO: 27 - protein, 117 aa, synthetic construct
  REGION 1..117: note = Synthetic
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDMIEQQWWYWGQGTQVTVSS

SEQ ID NO: 28 - protein, 117 aa, synthetic construct
  REGION 1..117: note = Synthetic
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDTHRGVYWYWGQGTQVTVSS

SEQ ID NO: 29 - protein, 117 aa, synthetic construct
  REGION 1..117: note = Synthetic
EVQLVESGGGLVQAGESLRLSCAASGSIASIKTMAWYRQAPGSQRELVAAINYIKEIYYADSVKGRFTISRDNAKTTVYLQMNSLRPEDTAVYYCVVDVTKEDYWYWGQGTQVTVSS

SEQ ID NO: 30 - protein, 117 aa, synthetic construct
  REGION 1..117: note = Synthetic
EVQLVESGGGLVQAGESLRLSCAASGSIASINSMAWYRQAPGSQRELVAAISNAREVYYADSVKGRFTISRDNAKTTVYLQMNSLRPEDTAVYYCVVDVYFQEYWYWGQGTQVTVSS

SEQ ID NO: 31 - protein, 117 aa, synthetic construct
  REGION 1..117: note = Synthetic
EVQLVESGGGLVQAGESLRLSCAASGSIASINTMAWYRQAPGSQRELVAAINSISRTYYADSVKGRFTISRDNAKTTVYLQMNSLRPEDTAVYYCVVDVTKEDYWYWGQGTQVTVSS

SEQ ID NO: 32 - protein, 117 aa, synthetic construct
  REGION 1..117: note = Synthetic
EVQLVESGGGLVQAGESLRLSCAASGSIASIKTMAWYRQAPGSQRELVTAIASDNRKYYADSVKGRFTISRDNAKTTVYLQMNSLRPEDTAVYYCVVDVTKEDYWYWGQGTQVTVSS

SEQ ID NO: 33 - protein, 117 aa, synthetic construct
  REGION 1..117: note = Synthetic
EVQLVESGGGLVQAGESLRLSCAASGSIASIRTMAWYRQAPGSQRELVAAISSGREVYYADSVKGRFTISRDNAKTTVYLQMNSLRPEDTAVYYCVVDMYWQDYWWWGQGTQVTVSS

SEQ ID NO: 34 - protein, 117 aa, synthetic construct
  REGION 1..117: note = Synthetic
EVQLVESGGGLVQAGESLRLSCAASGSIASINSMGWYRQAPGSQRELVAAISDRSEKYYADSVKGRFTISRDNAKTTVYLQMNSLRPEDTAVYYCVVDHHHSDWWTWGQGTQVTVSS

SEQ ID NO: 35 - protein, 16 aa, Homo sapiens
GKHLCPSPLFPGPSKP

SEQ ID NO: 36 - protein, 15 aa, Homo sapiens
KGKHLCPSPLFPGPS

SEQ ID NO: 37 - protein, 19 aa, Homo sapiens
HVKGKHLCPSPLFPGPSKP

SEQ ID NO: 38 - protein, 14 aa, Homo sapiens
DKTHTCPPCPAPEL

SEQ ID NO: 39 - protein, 227 aa, synthetic construct
  REGION 1..227: note = Synthetic
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 117 aa, synthetic construct
  REGION 1..117: note = Synthetic
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSS

SEQ ID NO: 41 - protein, 18 aa, Homo sapiens
MLRLLLALNLFPSIQVTG

SEQ ID NO: 42 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 43 - protein, 202 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 44 - DNA, 663 bp, Homo sapiens
  source 1..663: mol_type = other DNA
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctga

SEQ ID NO: 45 - protein, 152 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 46 - protein, 134 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 47 - protein, 110 aa, Homo sapiens
MLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKG

SEQ ID NO: 48 - protein, 14 aa, synthetic construct
  REGION 1..14: note = Synthetic
  SITE 14: note = X - X is A or T
WYRQAPGSQRELVX

SEQ ID NO: 49 - protein, 14 aa, synthetic construct
  REGION 1..14: note = Synthetic
WYRQAPGSQRELVT

SEQ ID NO: 50 - protein, 22 aa, Homo sapiens
EPKSCDKTHTCPPCPAPELLGG

SEQ ID NO: 51 - protein, 18 aa, synthetic construct
  REGION 1..18: note = Synthetic
EPKCCVECPPCPAPPAAA

SEQ ID NO: 52 - protein, 19 aa, synthetic construct
  REGION 1..19: note = Synthetic
ESKYGPPCPPCPAPEFLGG

SEQ ID NO: 53 - protein, 70 aa, synthetic construct
  REGION 1..70: note = Synthetic
ELKTPLGDTTHTCPRCPEPKSCDTPPPCPRCPEPKSCDTPPPCPRCPEPKSCDTPPPCPRCPAPELLGGP

SEQ ID NO: 54 - protein, 5 aa, synthetic construct
  REGION 1..5: note = Synthetic
  VARIANT 3: note = X is P or R
CPXCP

SEQ ID NO: 55 - protein, 5 aa, Homo sapiens
CPPCP

SEQ ID NO: 56 - protein, 5 aa, Homo sapiens
CPRCP

SEQ ID NO: 57 - protein, 106 aa, Homo sapiens
LGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 58 - protein, 103 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 59 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 362 aa, synthetic construct
  REGION 1..362: note = Synthetic
EVQLVESGGGLVQAGESLRLSCAASGSIASINSMGWYRQAPGSQRELVAAINEKLLIYYADSVKGRFTISRDNAKTTVYLQMNSLRPEDTAVYYCVVDLYGSDYWDWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 362 aa, synthetic construct
  REGION 1..362: note = Synthetic
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDMIEQQWWYWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 362 aa, synthetic construct
  REGION 1..362: note = Synthetic
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDTHRGVYWYWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 362 aa, synthetic construct
  REGION 1..362: note = Synthetic
EVQLVESGGGLVQAGESLRLSCAASGSIASIKTMAWYRQAPGSQRELVAAINYIKEIYYADSVKGRFTISRDNAKTTVYLQMNSLRPEDTAVYYCVVDVTKEDYWYWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 362 aa, synthetic construct
  REGION 1..362: note = Synthetic
EVQLVESGGGLVQAGESLRLSCAASGSIASINSMAWYRQAPGSQRELVAAISNAREVYYADSVKGRFTISRDNAKTTVYLQMNSLRPEDTAVYYCVVDVYFQEYWYWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 362 aa, synthetic construct
  REGION 1..362: note = Synthetic
EVQLVESGGGLVQAGESLRLSCAASGSIASINTMAWYRQAPGSQRELVAAINSISRTYYADSVKGRFTISRDNAKTTVYLQMNSLRPEDTAVYYCVVDVTKEDYWYWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 362 aa, synthetic construct
  REGION 1..362: note = Synthetic
EVQLVESGGGLVQAGESLRLSCAASGSIASIKTMAWYRQAPGSQRELVTAIASDNRKYYADSVKGRFTISRDNAKTTVYLQMNSLRPEDTAVYYCVVDVTKEDYWYWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 362 aa, synthetic construct
  REGION 1..362: note = Synthetic
EVQLVESGGGLVQAGESLRLSCAASGSIASIRTMAWYRQAPGSQRELVAAISSGREVYYADSVKGRFTISRDNAKTTVYLQMNSLRPEDTAVYYCVVDMYWQDYWWWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 362 aa, synthetic construct
  REGION 1..362: note = Synthetic
EVQLVESGGGLVQAGESLRLSCAASGSIASINSMGWYRQAPGSQRELVAAISDRSEKYYADSVKGRFTISRDNAKTTVYLQMNSLRPEDTAVYYCVVDHHHSDWWTWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 117 aa, synthetic construct
  REGION 1..117: note = Synthetic
EVQLVESGGGLVQPGGSLRLSCAASGSIASIKTMAWYRQAPGKQRELVTAIASDNRKYYADSVKGRFTISRDNSKNTLYLQMNSLRAEDTAVYYCVVDVTKEDYWYWGQGTLVTVSS

SEQ ID NO: 71 - protein, 117 aa, synthetic construct
  REGION 1..117: note = Synthetic
EVQLVESGGGLVQPGGSLRLSCKASGSIASIKTMAWYRQAPGKGLELVTAIASDNRKYYADSVKGRFTISRDNSKTTVYLQMNSLRAEDTAVYYCVVDVTKEDYWYWGQGTLVTVSS

SEQ ID NO: 72 - protein, 117 aa, synthetic construct
  REGION 1..117: note = Synthetic
EVQLVESGGGLVQPGGSLRLSCAASGSTASIKTMAWYRQAPGKGLELVTAIASDNRKYYADSVKGRFTISRDNSKTTVYLQMNSLRAEDTAVYYCVVDVTKEDYWYWGQGTLVTVSS

SEQ ID NO: 73 - protein, 117 aa, synthetic construct
  REGION 1..117: note = Synthetic
EVQLVESGGGLVQPGGSLRLSCKASGSTASIKTMAWYRQAPGKGLELVTAIASDNRKYYADSVKGRFTISRDNSKTTVYLQMNSLRAEDTAVYYCVVDVTKEDYWYWGQGTLVTVSS

SEQ ID NO: 74 - protein, 117 aa, synthetic construct
  REGION 1..117: note = Synthetic
EVQLVESGGGLVQPGGSLRLSCAASGSIASIKTMAWYRQAPGKGRELVTAIASDNRKYYADSVKGRFTISRDNSKTTVYLQMNSLRAEDTAVYYCVVDVTKEDYWYWGQGTLVTVSS

SEQ ID NO: 75 - protein, 117 aa, synthetic construct
  REGION 1..117: note = Synthetic
EVQLVESGGGLVQPGESLRLSCAASGSIASIRTMAWYRQAPGSQRELVAAISSGREVYYADSVKGRFTISRDNAKTTVYLQMNSLRAEDTAVYYCVVDMYWQDYWWWGQGTQVTVSS

SEQ ID NO: 76 - protein, 117 aa, synthetic construct
  REGION 1..117: note = Synthetic
EVQLVESGGGLVQPGGSLRLSCKASGSIASIRTMAWYRQAPGKGLELVAAISSGREVYYADSVKGRFTISRDNSKTTVYLQMNSLRAEDTAVYYCVVDMYWQDYWWWGQGTLVTVSS

SEQ ID NO: 77 - protein, 117 aa, synthetic construct
  REGION 1..117: note = Synthetic
EVQLVESGGGLVQPGGSLRLSCKASGSTASIRTMAWYRQAPGKGLELVSAISSGREVYYADSVKGRFTISRDNSKTTVYLQMNSLRAEDTAVYYCVVDMYWQDYWWWGQGTLVTVSS

SEQ ID NO: 78 - protein, 117 aa, synthetic construct
  REGION 1..117: note = Synthetic
EVQLVESGGGLVQPGGSLRLSCAASGSIASIRTMAWYRQAPGKGLELVSAISSGREVYYADSVKGRFTISRDNSKTTVYLQMNSLRAEDTAVYYCVVDMYWQDYWWWGQGTLVTVSS

SEQ ID NO: 79 - protein, 30 aa, synthetic construct
  REGION 1..30: note = Synthetic
  VARIANT 1: note = X1 is E or Q
  VARIANT 14: note = X2 is A or P
  VARIANT 16: note = X3 is E or G
  VARIANT 23: note = X4 is A or K
  VARIANT 28: note = X5 is I, L or T
  VARIANT 29: note = X6 is A or F
XVQLVESGGGLVQXGXSLRLSCXASGSXXS

SEQ ID NO: 80 - protein, 14 aa, synthetic construct
  REGION 1..14: note = Synthetic
  VARIANT 8: note = X7 is S or K
  VARIANT 9: note = X8 is Q or G
  VARIANT 10: note = X9 is R or L
  VARIANT 12: note = X10 is L or R
  VARIANT 14: note = X11 is one of: A, S, or T
WYRQAPGXXXEXVX

SEQ ID NO: 81 - protein, 32 aa, synthetic construct
  REGION 1..32: note = Synthetic
  VARIANT 4: note = X11 is I or V
  VARIANT 9: note = X12 is A or S
  VARIANT 11: note = X13 is T or N
  VARIANT 13: note = X14 is V, M or L
  VARIANT 19: note = X15 is S or N
  VARIANT 21: note = X16 is R, K, or E
  VARIANT 22: note = X17 is P or A
  VARIANT 23: note = X18 is E or R
  VARIANT 25: note = X19 is T or A
  VARIANT 26: note = X20 is A or G
RFTXSRDNXKXTXYLQMNXLXXXDXXVYYCVV

SEQ ID NO: 82 - protein, 11 aa, synthetic construct
  REGION 1..11: note = Synthetic
  VARIANT 6: note = X21 is an Q or L
WGQGTXVTVSS

SEQ ID NO: 83 - protein, 13 aa, synthetic construct
  REGION 1..13: note = Synthetic
DKTHTCPPCPAPE

SEQ ID NO: 84 - protein, 19 aa, synthetic construct
  REGION 1..19: note = Synthetic
EPKCCVECPPCPAPPVAGP

SEQ ID NO: 85 - protein, 19 aa, synthetic construct
  REGION 1..19: note = Synthetic
ESKYGPPCPPCPAPEFEGG

SEQ ID NO: 86 - protein, 19 aa, synthetic construct
  REGION 1..19: note = Synthetic
ESKYGPPCPSCPAPEFLGG

SEQ ID NO: 87 - protein, 9 aa, synthetic construct
  REGION 1..9: note = Synthetic
CCVECPPCP

SEQ ID NO: 88 - protein, 5 aa, synthetic construct
  REGION 1..5: note = Synthetic
CPSCP

SEQ ID NO: 89 - protein, 103 aa, synthetic construct
  REGION 1..103: note = Synthetic
PSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 90 - protein, 107 aa, synthetic construct
  REGION 1..107: note = Synthetic
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 229 aa, synthetic construct
  REGION 1..229: note = Synthetic
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 229 aa, synthetic construct
  REGION 1..229: note = Synthetic
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 371 aa, synthetic construct
  REGION 1..371: note = Synthetic
EVQLVESGGGLVQAGESLRLSCAASGSIASIKTMAWYRQAPGSQRELVTAIASDNRKYYADSVKGRFTISRDNAKTTVYLQMNSLRPEDTAVYYCVVDVTKEDYWYWGQGTQVTVSSGGGGSGGGGSGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 371 aa, synthetic construct
  REGION 1..371: note = Synthetic
EVQLVESGGGLVQAGESLRLSCAASGSIASIRTMAWYRQAPGSQRELVAAISSGREVYYADSVKGRFTISRDNAKTTVYLQMNSLRPEDTAVYYCVVDMYWQDYWWWGQGTQVTVSSGGGGSGGGGSGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 118 aa, synthetic construct
BEVQLVESGGGLVQAGGSLRLSCAASGSLFSINAMAWYRQAPGKQRELVAAITSSGSTNYANSVKGRFTVSRDNAKNTMYLQMNSLKPEDTAVYYCVVDEYGSDYWIWGQGTQVTVSS

SEQ ID NO: 96 - protein, 118 aa, synthetic construct
CQVQLVESGGGLVQAGGSLRLSCAASGSIFSINAMGWYRQAPGKQRERVAAITSGGSTNYADSVKGRFTISRDNAKNTVYLQMNNLEPRDAGVYYCVVDLYGEDYWIWGQGTQVTVSS

SEQ ID NO: 97 - protein, 31 aa, synthetic construct
GGGGSAEAAAKEAAAKEAAAKAAAGSGGGGS

SEQ ID NO: 98 - protein, 5 aa, synthetic construct
INAMA

SEQ ID NO: 99 - protein, 16 aa, synthetic construct
AITSSGSTNYANSVKG

SEQ ID NO: 100 - protein, 9 aa, synthetic construct
DEYGSDYWI

SEQ ID NO: 101 - protein, 16 aa, synthetic construct
AITSGGSTNYADSVKG

SEQ ID NO: 102 - protein, 9 aa, synthetic construct
DLYGEDYWI

SEQ ID NO: 103 - protein, 242 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAGGGGSEVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSS

SEQ ID NO: 104 - protein, 247 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAGGGGSGGGGSEVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSS

SEQ ID NO: 105 - protein, 257 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAGGGGSGGGGSGGGGSGGGGSEVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSS

SEQ ID NO: 106 - protein, 257 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAGGGKSGGGKSGGGKSGGGKSEVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSS

SEQ ID NO: 107 - protein, 257 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAGGGESGGGESGGGESGGGESEVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSS

SEQ ID NO: 108 - protein, 268 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAGGGGSAEAAAKEAAAKEAAAKAAAGSGGGGSEVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSS

SEQ ID NO: 109 - protein, 23 aa, synthetic construct
AAAGGGGSGGGGSGGGGSGGGGS

SEQ ID NO: 110 - protein, 23 aa, synthetic construct
AAAGGGKSGGGKSGGGKSGGGKS

SEQ ID NO: 111 - protein, 23 aa, synthetic construct
AAAGGGESGGGESGGGESGGGES

SEQ ID NO: 112 - protein, 34 aa, synthetic construct
AAAGGGGSAEAAAKEAAAKEAAAKAAAGSGGGGS

SEQ ID NO: 113 - protein, 118 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSC

SEQ ID NO: 114 - protein, 131 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSDKTHTCPPCPAPEL

SEQ ID NO: 115 - protein, 344 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 362 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 372 aa, synthetic construct
EVQLVESGGGLVQAGESLRLSCAASGSIASINAMGWYRQAPGSQRELVAAISGGGDTYYADSVKGRFTISRDNAKTTVYLQMNSLRPEDTAVYYCVVDLYGSDYWDWGQGTQVTVSSAAAGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 5 aa, synthetic construct
GGGGS

SEQ ID NO: 119 - protein, 8 aa, synthetic construct
AAAGGGGS